SEQUENCE LISTING (418 sequences)

SEQ ID NO: 1 - protein, 5 aa, Oryctolagus cuniculus
SSNAI

SEQ ID NO: 2 - protein, 11 aa, Oryctolagus cuniculus
QASQSIGSYLA

SEQ ID NO: 3 - protein, 16 aa, Oryctolagus cuniculus
GTIYADDNTYYANWAR

SEQ ID NO: 4 - protein, 7 aa, Oryctolagus cuniculus
RASTLAS

SEQ ID NO: 5 - protein, 8 aa, Oryctolagus cuniculus
KGASYYPL

SEQ ID NO: 6 - protein, 12 aa, Oryctolagus cuniculus
LGWHTYTDDGTH

SEQ ID NO: 7 - protein, 5 aa, Oryctolagus cuniculus
SSNAI

SEQ ID NO: 8 - protein, 12 aa, Oryctolagus cuniculus
QASQSIDNRYLS

SEQ ID NO: 9 - protein, 16 aa, Oryctolagus cuniculus
GYMDPERNIYYANWSK

SEQ ID NO: 10 - protein, 7 aa, Oryctolagus cuniculus
KASTLAS

SEQ ID NO: 11 - protein, 8 aa, Oryctolagus cuniculus
RGVTYYSM

SEQ ID NO: 12 - protein, 12 aa, Oryctolagus cuniculus
QGGYYGNSYVGA

SEQ ID NO: 13 - protein, 5 aa, Oryctolagus cuniculus
SSNAI

SEQ ID NO: 14 - protein, 11 aa, Oryctolagus cuniculus
QASESISTRLA

SEQ ID NO: 15 - protein, 16 aa, Oryctolagus cuniculus
GIIYASGDTYYASWAK

SEQ ID NO: 16 - protein, 7 aa, Oryctolagus cuniculus
SASTLPS

SEQ ID NO: 17 - protein, 8 aa, Oryctolagus cuniculus
RGYTTLYF

SEQ ID NO: 18 - protein, 11 aa, Oryctolagus cuniculus
QGGYSSGAGTA

SEQ ID NO: 19 - protein, 5 aa, Oryctolagus cuniculus
SRYHM

SEQ ID NO: 20 - protein, 11 aa, Oryctolagus cuniculus
QASENIYSSLA

SEQ ID NO: 21 - protein, 16 aa, Oryctolagus cuniculus
GIIYVSDNTYYATWAK

SEQ ID NO: 22 - protein, 7 aa, Oryctolagus cuniculus
EASNLES

SEQ ID NO: 23 - protein, 10 aa, Oryctolagus cuniculus
RVGSFWSSKL

SEQ ID NO: 24 - protein, 12 aa, Oryctolagus cuniculus
QSTYFGNSYVFA

SEQ ID NO: 25 - protein, 5 aa, Oryctolagus cuniculus
SSNAV

SEQ ID NO: 26 - protein, 12 aa, Oryctolagus cuniculus
QSSQSVYSNWLS

SEQ ID NO: 27 - protein, 16 aa, Oryctolagus cuniculus
GVISPGDDIYYANWAK

SEQ ID NO: 28 - protein, 7 aa, Oryctolagus cuniculus
QASKVPS

SEQ ID NO: 29 - protein, 8 aa, Oryctolagus cuniculus
RGFSYSAL

SEQ ID NO: 30 - protein, 12 aa, Oryctolagus cuniculus
QGTYDGSGWSNA

SEQ ID NO: 31 - protein, 5 aa, Oryctolagus cuniculus
SSNAM

SEQ ID NO: 32 - protein, 13 aa, Oryctolagus cuniculus
QASESVGNNNYLS

SEQ ID NO: 33 - protein, 16 aa, Oryctolagus cuniculus
GIISNSGSTYYASWAK

SEQ ID NO: 34 - protein, 7 aa, Oryctolagus cuniculus
DASRLAS

SEQ ID NO: 35 - protein, 8 aa, Oryctolagus cuniculus
RGFRYPNP

SEQ ID NO: 36 - protein, 12 aa, Oryctolagus cuniculus
LGGYVSSGWYGA

SEQ ID NO: 37 - protein, 5 aa, Oryctolagus cuniculus
SSNAL

SEQ ID NO: 38 - protein, 13 aa, Oryctolagus cuniculus
QASQSVHNNNYLS

SEQ ID NO: 39 - protein, 16 aa, Oryctolagus cuniculus
ASIYAGGDTYYATWAK

SEQ ID NO: 40 - protein, 7 aa, Oryctolagus cuniculus
QASKLAS

SEQ ID NO: 41 - protein, 8 aa, Oryctolagus cuniculus
RGAMTYSL

SEQ ID NO: 42 - protein, 14 aa, Oryctolagus cuniculus
QSYYYSGSSGAVNS

SEQ ID NO: 43 - protein, 5 aa, Oryctolagus cuniculus
SSNAI

SEQ ID NO: 44 - protein, 11 aa, Oryctolagus cuniculus
QASQSISNLIS

SEQ ID NO: 45 - protein, 16 aa, Oryctolagus cuniculus
GVIYAGGGAFYANWAK

SEQ ID NO: 46 - protein, 7 aa, Oryctolagus cuniculus
KASTLAS

SEQ ID NO: 47 - protein, 8 aa, Oryctolagus cuniculus
RGYTYLAF

SEQ ID NO: 48 - protein, 12 aa, Oryctolagus cuniculus
QGSAYGTSDVCA

SEQ ID NO: 49 - protein, 5 aa, Oryctolagus cuniculus
SSNAM

SEQ ID NO: 50 - protein, 13 aa, Oryctolagus cuniculus
QSSQSILSDNYLA

SEQ ID NO: 51 - protein, 16 aa, Oryctolagus cuniculus
GAIDANGSPYYTNWAK

SEQ ID NO: 52 - protein, 7 aa, Oryctolagus cuniculus
QASKLVS

SEQ ID NO: 53 - protein, 8 aa, Oryctolagus cuniculus
RGYTRLDL

SEQ ID NO: 54 - protein, 12 aa, Oryctolagus cuniculus
QGAYDSSDWYGA

SEQ ID NO: 55 - protein, 5 aa, Oryctolagus cuniculus
NNYAM

SEQ ID NO: 56 - protein, 13 aa, Oryctolagus cuniculus
QASQSVYNGNELS

SEQ ID NO: 57 - protein, 16 aa, Oryctolagus cuniculus
GFINSGGSAYYASWAK

SEQ ID NO: 58 - protein, 7 aa, Oryctolagus cuniculus
AASILAS

SEQ ID NO: 59 - protein, 8 aa, Oryctolagus cuniculus
RGVPKMDL

SEQ ID NO: 60 - protein, 12 aa, Oryctolagus cuniculus
AGYQSSVIDDIG

SEQ ID NO: 61 - protein, 5 aa, Oryctolagus cuniculus
NNYAM

SEQ ID NO: 62 - protein, 13 aa, Oryctolagus cuniculus
QASQSVYNNNELS

SEQ ID NO: 63 - protein, 16 aa, Oryctolagus cuniculus
GFINTGDRAYYASWAK

SEQ ID NO: 64 - protein, 7 aa, Oryctolagus cuniculus
AASYVAS

SEQ ID NO: 65 - protein, 8 aa, Oryctolagus cuniculus
RGVPAMGL

SEQ ID NO: 66 - protein, 12 aa, Oryctolagus cuniculus
AGYESSGIDDIG

SEQ ID NO: 67 - protein, 5 aa, Oryctolagus cuniculus
SSNAM

SEQ ID NO: 68 - protein, 12 aa, Oryctolagus cuniculus
QSSQSVGSNYLS

SEQ ID NO: 69 - protein, 16 aa, Oryctolagus cuniculus
GIIYASGSTYYASWAK

SEQ ID NO: 70 - protein, 7 aa, Oryctolagus cuniculus
DASTLAS

SEQ ID NO: 71 - protein, 8 aa, Oryctolagus cuniculus
RGFARLPL

SEQ ID NO: 72 - protein, 12 aa, Oryctolagus cuniculus
QGSYYSSDWYGA

SEQ ID NO: 73 - protein, 6 aa, Oryctolagus cuniculus
NTNYYM

SEQ ID NO: 74 - protein, 11 aa, Oryctolagus cuniculus
QASESISNYLA

SEQ ID NO: 75 - protein, 17 aa, Oryctolagus cuniculus
ACSYTTSGSTYYATWAK

SEQ ID NO: 76 - protein, 7 aa, Oryctolagus cuniculus
RASTLES

SEQ ID NO: 77 - protein, 10 aa, Oryctolagus cuniculus
KYGAGYTYNL

SEQ ID NO: 78 - protein, 12 aa, Oryctolagus cuniculus
QQGYSNTNLDNI

SEQ ID NO: 79 - protein, 5 aa, Oryctolagus cuniculus
SSNAI

SEQ ID NO: 80 - protein, 13 aa, Oryctolagus cuniculus
QASQSVYNNNYLS

SEQ ID NO: 81 - protein, 16 aa, Oryctolagus cuniculus
GWIDATGSAYYATWAK

SEQ ID NO: 82 - protein, 7 aa, Oryctolagus cuniculus
DTSTLAS

SEQ ID NO: 83 - protein, 8 aa, Oryctolagus cuniculus
RGFRYSAF

SEQ ID NO: 84 - protein, 12 aa, Oryctolagus cuniculus
AGTYSTSDWSVA

SEQ ID NO: 85 - protein, 5 aa, Oryctolagus cuniculus
STYFM

SEQ ID NO: 86 - protein, 11 aa, Oryctolagus cuniculus
QASQSIHNYLA

SEQ ID NO: 87 - protein, 16 aa, Oryctolagus cuniculus
GWINTNDKIYYASWAK

SEQ ID NO: 88 - protein, 7 aa, Oryctolagus cuniculus
SASNLAS

SEQ ID NO: 89 - protein, 12 aa, Oryctolagus cuniculus
SPYPRYASGLNL

SEQ ID NO: 90 - protein, 12 aa, Oryctolagus cuniculus
QCTYYGSSYENT

SEQ ID NO: 91 - protein, 5 aa, Oryctolagus cuniculus
SRYHM

SEQ ID NO: 92 - protein, 13 aa, Oryctolagus cuniculus
QSSESVSNNNWLS

SEQ ID NO: 93 - protein, 16 aa, Oryctolagus cuniculus
ATTHIDGGVYYAIWAK

SEQ ID NO: 94 - protein, 7 aa, Oryctolagus cuniculus
QASKLAS

SEQ ID NO: 95 - protein, 5 aa, Oryctolagus cuniculus
RKFDL

SEQ ID NO: 96 - protein, 12 aa, Oryctolagus cuniculus
QGGYYDSGWYYA

SEQ ID NO: 97 - protein, 5 aa, Oryctolagus cuniculus
SSNAI

SEQ ID NO: 98 - protein, 13 aa, Oryctolagus cuniculus
QASQSVYYNNYLA

SEQ ID NO: 99 - protein, 16 aa, Oryctolagus cuniculus
GFIDSEGSASYASWAN

SEQ ID NO: 100 - protein, 7 aa, Oryctolagus cuniculus
DTSKLAS

SEQ ID NO: 101 - protein, 8 aa, Oryctolagus cuniculus
RGFRYLPL

SEQ ID NO: 102 - protein, 13 aa, Oryctolagus cuniculus
QGTYYSSGWYWNA

SEQ ID NO: 103 - protein, 5 aa, Oryctolagus cuniculus
SSNAI

SEQ ID NO: 104 - protein, 11 aa, Oryctolagus cuniculus
QASQSINNFLS

SEQ ID NO: 105 - protein, 16 aa, Oryctolagus cuniculus
GVIYAGGGAFYASWAK

SEQ ID NO: 106 - protein, 7 aa, Oryctolagus cuniculus
KASTLAS

SEQ ID NO: 107 - protein, 8 aa, Oryctolagus cuniculus
RGYTYLAF

SEQ ID NO: 108 - protein, 12 aa, Oryctolagus cuniculus
QGSAYGTSDVCA

SEQ ID NO: 109 - protein, 5 aa, Oryctolagus cuniculus
SSNAL

SEQ ID NO: 110 - protein, 13 aa, Oryctolagus cuniculus
QASQSVHNNNYLS

SEQ ID NO: 111 - protein, 16 aa, Oryctolagus cuniculus
ASIYAGGDTYYATWAK

SEQ ID NO: 112 - protein, 7 aa, Oryctolagus cuniculus
QASKLAS

SEQ ID NO: 113 - protein, 8 aa, Oryctolagus cuniculus
RGAMTYSL

SEQ ID NO: 114 - protein, 14 aa, Oryctolagus cuniculus
QSYYYSGCSGAVNS

SEQ ID NO: 115 - protein, 5 aa, Oryctolagus cuniculus
SSNAV

SEQ ID NO: 116 - protein, 11 aa, Oryctolagus cuniculus
QASESISSWLA

SEQ ID NO: 117 - protein, 16 aa, Oryctolagus cuniculus
GVISVSGNIYYANWAR

SEQ ID NO: 118 - protein, 7 aa, Oryctolagus cuniculus
LASTLAS

SEQ ID NO: 119 - protein, 5 aa, Oryctolagus cuniculus
RPWDL

SEQ ID NO: 120 - protein, 12 aa, Oryctolagus cuniculus
QCSSYTSGYVAA

SEQ ID NO: 121 - protein, 5 aa, Oryctolagus cuniculus
NSFHM

SEQ ID NO: 122 - protein, 13 aa, Oryctolagus cuniculus
QASQSVYNNNWLS

SEQ ID NO: 123 - protein, 16 aa, Oryctolagus cuniculus
GVIHPNDATYYASWAK

SEQ ID NO: 124 - protein, 7 aa, Oryctolagus cuniculus
RASTLAS

SEQ ID NO: 125 - protein, 12 aa, Oryctolagus cuniculus
RDLAGYSTGGSF

SEQ ID NO: 126 - protein, 12 aa, Oryctolagus cuniculus
AAYKSWSNDDFG

SEQ ID NO: 127 - protein, 5 aa, Oryctolagus cuniculus
SSVAM

SEQ ID NO: 128 - protein, 11 aa, Oryctolagus cuniculus
RASEDIESYLA

SEQ ID NO: 129 - protein, 16 aa, Oryctolagus cuniculus
GVISTSGNKYYATWAK

SEQ ID NO: 130 - protein, 7 aa, Oryctolagus cuniculus
RASKLAS

SEQ ID NO: 131 - protein, 5 aa, Oryctolagus cuniculus
RAWNL

SEQ ID NO: 132 - protein, 13 aa, Oryctolagus cuniculus
QCTTYTSTYVGGG

SEQ ID NO: 133 - protein, 5 aa, Oryctolagus cuniculus
SSNAM

SEQ ID NO: 134 - protein, 13 aa, Oryctolagus cuniculus
QSSQSVVSNNRLS

SEQ ID NO: 135 - protein, 16 aa, Oryctolagus cuniculus
GFIDAGGSAYYATWVN

SEQ ID NO: 136 - protein, 7 aa, Oryctolagus cuniculus
LASTLPS

SEQ ID NO: 137 - protein, 8 aa, Oryctolagus cuniculus
KGLSWSDF

SEQ ID NO: 138 - protein, 12 aa, Oryctolagus cuniculus
QGTYGSSSYYGA

SEQ ID NO: 139 - protein, 5 aa, Oryctolagus cuniculus
SSFHM

SEQ ID NO: 140 - protein, 13 aa, Oryctolagus cuniculus
QSSKSVCNNDWLS

SEQ ID NO: 141 - protein, 16 aa, Oryctolagus cuniculus
GVIHPNDITYYASWAK

SEQ ID NO: 142 - protein, 7 aa, Oryctolagus cuniculus
RASTLAS

SEQ ID NO: 143 - protein, 12 aa, Oryctolagus cuniculus
RDLTGGTTGGRL

SEQ ID NO: 144 - protein, 12 aa, Oryctolagus cuniculus
AGYASWNNDDFG

SEQ ID NO: 145 - protein, 5 aa, Oryctolagus cuniculus
SSYWM

SEQ ID NO: 146 - protein, 12 aa, Oryctolagus cuniculus
SSQSVGSGNILS

SEQ ID NO: 147 - protein, 16 aa, Oryctolagus cuniculus
GIISTSENIYYATWAK

SEQ ID NO: 148 - protein, 7 aa, Oryctolagus cuniculus
QASKLAS

SEQ ID NO: 149 - protein, 5 aa, Oryctolagus cuniculus
RWSDL

SEQ ID NO: 150 - protein, 13 aa, Oryctolagus cuniculus
LGSYGCSSADCAA

SEQ ID NO: 151 - protein, 5 aa, Oryctolagus cuniculus
SSNAI

SEQ ID NO: 152 - protein, 12 aa, Oryctolagus cuniculus
QASQSISSSYLS

SEQ ID NO: 153 - protein, 16 aa, Oryctolagus cuniculus
GIIDSNGSTYYASWAK

SEQ ID NO: 154 - protein, 7 aa, Oryctolagus cuniculus
KASTLAS

SEQ ID NO: 155 - protein, 8 aa, Oryctolagus cuniculus
RGAIYPAL

SEQ ID NO: 156 - protein, 13 aa, Oryctolagus cuniculus
LYAYFGGSTAEHT

SEQ ID NO: 157 - protein, 5 aa, Oryctolagus cuniculus
SRYYM

SEQ ID NO: 158 - protein, 13 aa, Oryctolagus cuniculus
QSSESVSNNNWLS

SEQ ID NO: 159 - protein, 16 aa, Oryctolagus cuniculus
ATTHIDGGVYYANWAK

SEQ ID NO: 160 - protein, 7 aa, Oryctolagus cuniculus
AASKLAS

SEQ ID NO: 161 - protein, 5 aa, Oryctolagus cuniculus
RKFDL

SEQ ID NO: 162 - protein, 12 aa, Oryctolagus cuniculus
QGGYYDSGWYYA

SEQ ID NO: 163 - protein, 5 aa, Oryctolagus cuniculus
SSNAI

SEQ ID NO: 164 - protein, 13 aa, Oryctolagus cuniculus
QASESVGSNNRLS

SEQ ID NO: 165 - protein, 16 aa, Oryctolagus cuniculus
GIIDAPGSTYYASWAK

SEQ ID NO: 166 - protein, 7 aa, Oryctolagus cuniculus
EASKLPS

SEQ ID NO: 167 - protein, 8 aa, Oryctolagus cuniculus
RNYAYFAL

SEQ ID NO: 168 - protein, 12 aa, Oryctolagus cuniculus
LGWHASTDDGWA

SEQ ID NO: 169 - protein, 5 aa, Oryctolagus cuniculus
SSNAI

SEQ ID NO: 170 - protein, 12 aa, Oryctolagus cuniculus
QASQSISSNYYA

SEQ ID NO: 171 - protein, 16 aa, Oryctolagus cuniculus
GIIWSGGDTDYATWAK

SEQ ID NO: 172 - protein, 7 aa, Oryctolagus cuniculus
KASTLAS

SEQ ID NO: 173 - protein, 8 aa, Oryctolagus cuniculus
KGATYSAL

SEQ ID NO: 174 - protein, 12 aa, Oryctolagus cuniculus
QGFDYGNSNVGA

SEQ ID NO: 175 - protein, 5 aa, Oryctolagus cuniculus
SSNAM

SEQ ID NO: 176 - protein, 12 aa, Oryctolagus cuniculus
QSSQSVYIDRLA

SEQ ID NO: 177 - protein, 16 aa, Oryctolagus cuniculus
GYIWSGGNTDYASWAK

SEQ ID NO: 178 - protein, 7 aa, Oryctolagus cuniculus
QASKLPS

SEQ ID NO: 179 - protein, 8 aa, Oryctolagus cuniculus
RGGSYFPF

SEQ ID NO: 180 - protein, 14 aa, Oryctolagus cuniculus
AGFYDSGSGTYTLA

SEQ ID NO: 181 - protein, 5 aa, Oryctolagus cuniculus
SSNAI

SEQ ID NO: 182 - protein, 11 aa, Oryctolagus cuniculus
QASQSISNILA

SEQ ID NO: 183 - protein, 16 aa, Oryctolagus cuniculus
GVIDAGGSTYFASWAK

SEQ ID NO: 184 - protein, 7 aa, Oryctolagus cuniculus
SASTLAS

SEQ ID NO: 185 - protein, 8 aa, Oryctolagus cuniculus
RGWSRHDF

SEQ ID NO: 186 - protein, 11 aa, Oryctolagus cuniculus
QGYDSTVGVGA

SEQ ID NO: 187 - protein, 5 aa, Oryctolagus cuniculus
SRYHM

SEQ ID NO: 188 - protein, 11 aa, Oryctolagus cuniculus
QASENIYNNLA

SEQ ID NO: 189 - protein, 16 aa, Oryctolagus cuniculus
GIIYVSDDSYYASWAK

SEQ ID NO: 190 - protein, 7 aa, Oryctolagus cuniculus
RASTLES

SEQ ID NO: 191 - protein, 10 aa, Oryctolagus cuniculus
RVGSVWSSKL

SEQ ID NO: 192 - protein, 12 aa, Oryctolagus cuniculus
QSTYFGGSYVFA

SEQ ID NO: 193 - protein, 5 aa, Oryctolagus cuniculus
SSNAI

SEQ ID NO: 194 - protein, 13 aa, Oryctolagus cuniculus
QASESVASNDRLA

SEQ ID NO: 195 - protein, 16 aa, Oryctolagus cuniculus
GTIYATDSTSYASWAK

SEQ ID NO: 196 - protein, 7 aa, Oryctolagus cuniculus
QASTLAS

SEQ ID NO: 197 - protein, 8 aa, Oryctolagus cuniculus
LGASYSAL

SEQ ID NO: 198 - protein, 12 aa, Oryctolagus cuniculus
AGYKSSSTDGNA

SEQ ID NO: 199 - protein, 5 aa, Oryctolagus cuniculus
SSYDM

SEQ ID NO: 200 - protein, 11 aa, Oryctolagus cuniculus
QASESIGSWLA

SEQ ID NO: 201 - protein, 16 aa, Oryctolagus cuniculus
GVIATGGRRDYASWAK

SEQ ID NO: 202 - protein, 7 aa, Oryctolagus cuniculus
SASTLAF

SEQ ID NO: 203 - protein, 10 aa, Oryctolagus cuniculus
RYSDSDGYAL

SEQ ID NO: 204 - protein, 11 aa, Oryctolagus cuniculus
QSNYYSTSGHA

SEQ ID NO: 205 - protein, 5 aa, Oryctolagus cuniculus
SNYWM

SEQ ID NO: 206 - protein, 13 aa, Oryctolagus cuniculus
QASQSVYNNNYLS

SEQ ID NO: 207 - protein, 16 aa, Oryctolagus cuniculus
GTINYGGSTYYASWAK

SEQ ID NO: 208 - protein, 7 aa, Oryctolagus cuniculus
AASTLAS

SEQ ID NO: 209 - protein, 10 aa, Oryctolagus cuniculus
RDNGAYTFDS

SEQ ID NO: 210 - protein, 13 aa, Oryctolagus cuniculus
LGKSSCSYDDCRA

SEQ ID NO: 211 - protein, 6 aa, Oryctolagus cuniculus
SYYSWA

SEQ ID NO: 212 - protein, 11 aa, Oryctolagus cuniculus
QASQSIRSDLA

SEQ ID NO: 213 - protein, 18 aa, Oryctolagus cuniculus
ACIDGGGSRATYYASWAK

SEQ ID NO: 214 - protein, 7 aa, Oryctolagus cuniculus
KASTLAS

SEQ ID NO: 215 - protein, 13 aa, Oryctolagus cuniculus
RSDYNGYISYFDL

SEQ ID NO: 216 - protein, 10 aa, Oryctolagus cuniculus
QSYYHSSSTA

SEQ ID NO: 217 - protein, 5 aa, Oryctolagus cuniculus
SSNAI

SEQ ID NO: 218 - protein, 12 aa, Oryctolagus cuniculus
QASQSIASRYCS

SEQ ID NO: 219 - protein, 16 aa, Oryctolagus cuniculus
GYIDANTNAYYASWAK

SEQ ID NO: 220 - protein, 7 aa, Oryctolagus cuniculus
KASTLAS

SEQ ID NO: 221 - protein, 8 aa, Oryctolagus cuniculus
RGVTYYPM

SEQ ID NO: 222 - protein, 12 aa, Oryctolagus cuniculus
QGGYYGDSYVGA

SEQ ID NO: 223 - protein, 5 aa, Oryctolagus cuniculus
SSNAI

SEQ ID NO: 224 - protein, 12 aa, Oryctolagus cuniculus
QASQSINGNYLA

SEQ ID NO: 225 - protein, 16 aa, Oryctolagus cuniculus
GAIYSDDNTYYANWAK

SEQ ID NO: 226 - protein, 7 aa, Oryctolagus cuniculus
KASTLTS

SEQ ID NO: 227 - protein, 8 aa, Oryctolagus cuniculus
RGASRFDF

SEQ ID NO: 228 - protein, 12 aa, Oryctolagus cuniculus
QYTDYGSTYVGA

SEQ ID NO: 229 - protein, 5 aa, Oryctolagus cuniculus
SSANI

SEQ ID NO: 230 - protein, 12 aa, Oryctolagus cuniculus
QASQSIGSRYWS

SEQ ID NO: 231 - protein, 16 aa, Oryctolagus cuniculus
GYIDANTNAYYASWAK

SEQ ID NO: 232 - protein, 7 aa, Oryctolagus cuniculus
KASTLAS

SEQ ID NO: 233 - protein, 8 aa, Oryctolagus cuniculus
RGVTYYPM

SEQ ID NO: 234 - protein, 12 aa, Oryctolagus cuniculus
QGGYYGDSYVGA

SEQ ID NO: 235 - protein, 5 aa, Oryctolagus cuniculus
SSNAM

SEQ ID NO: 236 - protein, 12 aa, Oryctolagus cuniculus
QASQSISSSYLA

SEQ ID NO: 237 - protein, 16 aa, Oryctolagus cuniculus
GIIYASDSTYYASWAK

SEQ ID NO: 238 - protein, 7 aa, Oryctolagus cuniculus
KASTLAS

SEQ ID NO: 239 - protein, 8 aa, Oryctolagus cuniculus
RGATYIPL

SEQ ID NO: 240 - protein, 12 aa, Oryctolagus cuniculus
QCTDYGSSYVGT

SEQ ID NO: 241 - protein, 5 aa, Oryctolagus cuniculus
SSNTM

SEQ ID NO: 242 - protein, 11 aa, Oryctolagus cuniculus
QASQSIDSYLS

SEQ ID NO: 243 - protein, 16 aa, Oryctolagus cuniculus
GLIGPVSNTYYANWAK

SEQ ID NO: 244 - protein, 7 aa, Oryctolagus cuniculus
KASTLAS

SEQ ID NO: 245 - protein, 8 aa, Oryctolagus cuniculus
RGWFQYSF

SEQ ID NO: 246 - protein, 13 aa, Oryctolagus cuniculus
QGGYYSSSNNYIT

SEQ ID NO: 247 - protein, 112 aa, Oryctolagus cuniculus
QSLEESGGRLVTPGTPLTLTCTVSGFSLSSNAISWVRQAPGKGLEWIGTIYADDNTYYANWARGRFTISRTSTTVDLKITSPTTEDTATYFCAKGASYYPLWGPGTLVTVSS

SEQ ID NO: 248 - DNA, 336 bp, Oryctolagus cuniculus
cagtcgctggaggagtccgggggtcgcctggtcacgcctgggacacccctgacactcacctgcacagtctctggattctccctcagtagcaatgcaataagctgggtccgccaggctccagggaaggggctggaatggatcggaaccatttatgctgatgataacacatattacgcgaactgggcgagaggccggttcaccatctccagaacctcgaccacggtggatctgaaaatcaccagtccgacaaccgaggacacggccacctatttctgtgccaaaggtgcttcttattatcctttgtggggcccaggcaccctggtcaccgtctcctca

SEQ ID NO: 249 - protein, 109 aa, Oryctolagus cuniculus
VVMTQTPSSTSAAVEGTVTINCQASQSIGSYLAWFQQKPGQPPKLLIYRASTLASGVPSRFKGSGSGTQFTLTISGVQREDAATYYCLGWHTYTDDGTHFGGGTEVVVK

SEQ ID NO: 250 - DNA, 327 bp, Oryctolagus cuniculus
gtcgtgatgacccagactccatcctccacgtctgccgctgtggaaggcacagtcaccatcaattgccaggccagtcagagcattggtagctatttggcctggtttcagcagaaaccagggcagcctcccaagctcctgatctacagggcttccactctggcatctggggtcccatcgcggttcaaaggcagtggatctgggacacagttcactctcaccatcagcggcgtgcagcgtgaggatgctgccacttactactgtctaggctggcatacttatactgatgatggaactcatttcggcggagggaccgaggtggtggtcaaa

SEQ ID NO: 251 - protein, 112 aa, Oryctolagus cuniculus
QSVEESGGRLVTPGTPLTLTCTVSGFSLSSNAISWVRQAPGKGLEWIGYMDPERNIYYANWSKGRFTFSQTSTTVDLKIASPTSEDTATYFCARGVTYYSMWGPGTLVTVSS

SEQ ID NO: 252 - DNA, 336 bp, Oryctolagus cuniculus
cagtcggtggaggagtccgggggtcgcctggtcacgcctgggacacccctgacactcacctgcaccgtctctggattctccctcagtagcaatgcaataagctgggtccgccaggctccaggaaaggggctggagtggatcggatacatggatcctgagagaaacatatactacgcgaattggtcaaaaggccgattcaccttctcccaaacctcgaccacggtggatctgaaaatcgccagtccgacaagcgaggacacggccacctatttctgtgccagaggtgttacttattattcaatgtggggcccgggcaccctggtcaccgtctcctca

SEQ ID NO: 253 - protein, 111 aa, Oryctolagus cuniculus
DVVMTQTPASVSEPVGGTVTIKCQASQSIDNRYLSWYQQKPGQPPKLLIYKASTLASGVSSRFKGSGSGTEFTLTISDLECADAATYYCQGGYYGNSYVGAFGGGTEVVVK

SEQ ID NO: 254 - DNA, 333 bp, Oryctolagus cuniculus
gatgttgtgatgacccagactccagcctccgtgtctgaacctgtgggaggcacagtcaccatcaagtgccaggccagtcagagtattgataataggtacttatcctggtatcagcagaaaccagggcagcctcccaagctcctgatctacaaggcatccactctggcatctggggtctcatcgcggttcaaaggcagtggatctgggacagagttcactctcaccatcagcgacctggagtgtgccgatgctgccacttactactgtcaaggcggttattatggtaatagttatgttggcgctttcggcggagggaccgaggtggtggtcaaa

SEQ ID NO: 255 - protein, 113 aa, Oryctolagus cuniculus
QSVKESGGGLFKPTDTLTLTCTVSGFSLSSNAISWVRQAPGKGLEWIGIIYASGDTYYASWAKGRFTISKTSSTTVDLKMTSLTTEGTATYFCARGYTTLYFWGPGTLVTVSS

SEQ ID NO: 256 - DNA, 339 bp, Oryctolagus cuniculus
cagtcagtgaaggagtccgggggaggtctcttcaagccaacggataccctgacactcacctgcaccgtctctggattctccctcagtagtaatgcaataagctgggtccgccaggctccagggaaggggctggaatggatcggaatcatttatgctagtggtgacacatactacgcgagctgggcgaaaggccgattcaccatctccaaaacctcgtcgaccacggtggatctgaaaatgaccagtctgacaaccgagggcacggccacctatttctgtgccagaggatatactactctttacttctggggcccaggcaccctggtcaccgtctcctca

SEQ ID NO: 257 - protein, 109 aa, Oryctolagus cuniculus
QIVMTQTPASVSAAVGGTVTIKCQASESISTRLAWYQQKPGQPPKLLIYSASTLPSGVPSRFSGSGSGTDFTLTISGVQCDDAATYYCQGGYSSGAGTAFGGGTEVVVK

SEQ ID NO: 258 - DNA, 327 bp, Oryctolagus cuniculus
caaattgtgatgacccagactccagcctccgtgtctgcagctgtgggaggcacagtcaccatcaagtgccaggccagtgagagtattagtactaggttagcctggtatcagcagaaaccagggcagcctcccaagctcctgatctactctgcatccactctgccatctggggtcccatcgcggttcagtggcagtggatctgggacagacttcactctcaccatcagcggcgtgcagtgtgacgatgctgccacttactactgtcaaggcggttatagtagtggtgctggtactgctttcggcggagggaccgaggtggtggtcaaa

SEQ ID NO: 259 - protein, 114 aa, Oryctolagus cuniculus
QSLEESGGRLVTPGTPLTLTCTASGFDFSRYHMCWVRQAPGKGLEWIGIIYVSDNTYYATWAKGRFTISRTSTTVDLKITSPTTEDTATYFCVRVGSFWSSKLWGPGTLVTVSS

SEQ ID NO: 260 - DNA, 342 bp, Oryctolagus cuniculus
cagtcgctggaggagtccgggggtcgcctggtcacgcctgggacacccctgacactcacctgcacagcctctggattcgacttcagtaggtaccacatgtgctgggtccgccaggctccagggaaggggctggaatggatcggaatcatttatgttagtgataacacatactacgcgacctgggcaaaaggccgattcaccatctccagaacctcgaccacggtggatctgaaaatcaccagtccgacaaccgaggacacggccacctatttctgtgtcagagttggtagtttttggagcagtaagttgtggggcccaggcaccctggtcaccgtctcctca

SEQ ID NO: 261 - protein, 110 aa, Oryctolagus cuniculus
DVVMTQTPASVSEPVGGTVTINCQASENIYSSLAWYQQKPGQPPKLLIYEASNLESGVSSRFSGSGSGTEFTLTISDLECADAATYYCQSTYFGNSYVFAFGGGTEVVVK

SEQ ID NO: 262 - DNA, 330 bp, Oryctolagus cuniculus
gatgttgtgatgacccagactccagcctccgtgtctgaacctgtgggaggcacagtcaccatcaattgccaggccagtgagaacatttacagctctttagcctggtatcagcagaaaccagggcagcctcccaagctcctgatctatgaagcatccaatctagaatctggggtctcatcaaggttcagcggcagtggatctgggacagagttcactctcaccatcagcgacctggagtgtgccgatgctgccacttactactgtcaatctacttatttcggtaatagttatgtttttgctttcggcggagggaccgaggtggtggtcaaa

SEQ ID NO: 263 - protein, 112 aa, Oryctolagus cuniculus
QSVEESGGRLVTPGTPLTLTCTVSGFSLSSNAVNWVRQAPGEGLEWIGVISPGDDIYYANWAKGRFTISKTSTTVDLKITSPTTEDTATYFCARGFSYSALWGQGTLVTVSS

SEQ ID NO: 264 - DNA, 336 bp, Oryctolagus cuniculus
cagtcggtggaggagtccgggggtcgcctggtcacgcctgggacacccctgacactcacctgcaccgtctctggattctccctcagtagtaatgcagtgaactgggtccgccaggctccaggggaggggctggaatggatcggagtcattagtcctggtgatgacatatactacgcgaattgggcaaaaggccgattcaccatctccaaaacctcgaccacggtggatctgaaaatcaccagtccgacaaccgaggacacggccacctatttctgtgccagaggtttttcctattcagccttgtggggccaaggcaccctggtcaccgtctcctca

SEQ ID NO: 265 - protein, 110 aa, Oryctolagus cuniculus
QVLTQTASPVSAAVGGTVTINCQSSQSVYSNWLSWYQQKPGQRPKLLIYQASKVPSGVSSRFSGSGSGTQFILTISGVQCDDAATYYCQGTYDGSGWSNAFGGGTEVVVK

SEQ ID NO: 266 - DNA, 330 bp, Oryctolagus cuniculus
caagtgctgacccagactgcatcgcccgtgtctgccgctgtgggaggcacagtcaccatcaactgccagtccagtcagagtgtttatagtaactggctatcctggtatcagcagaaaccagggcagcgtcccaagctcctgatctaccaggcatccaaggtgccatctggggtctcatcgcggttcagcggcagtggatctgggacacagttcattctcaccatcagcggcgtgcagtgtgacgatgctgccacttactactgtcaaggcacttatgatggtagtggttggtctaatgctttcggcggagggaccgaggtggtggtcaaa

SEQ ID NO: 267 - protein, 113 aa, Oryctolagus cuniculus
QSVEESGGRLVTPGTPLTLTCTVSGIDLSSNAMTWVRQAPGEGLEWIGIISNSGSTYYASWAKGRFTISKTSSTTVDLKMTSLTTEDTATYFCARGFRYPNPWGPGTLVTVSS

SEQ ID NO: 268 - DNA, 339 bp, Oryctolagus cuniculus
cagtcggtggaggagtccgggggtcgcctggtcacgcctgggacacccctgacactcacctgcacagtctctggaatcgacctcagtagcaatgcaatgacctgggtccgccaggctccaggggaggggctggaatggatcggaatcattagtaatagtggtagcacatactacgcgagctgggcgaaaggccgattcaccatctccaaaacctcgtcgaccacggtggatctgaaaatgaccagtctgacaaccgaggacacggccacctatttctgtgccagaggttttagatatcctaatccctggggcccaggcaccctggtcaccgtctcctca

SEQ ID NO: 269 - protein, 111 aa, Oryctolagus cuniculus
QVLTQTPSSVSAAVGGTVTINCQASESVGNNNYLSWYQQRPGQPPKVLIYDASRLASGVSSRFKGSGSGTQFTLTISGVQCDDAATYYCLGGYVSSGWYGAFGGGTEVVVK

SEQ ID NO: 270 - DNA, 333 bp, Oryctolagus cuniculus
caagtgctgacccagactccatcctccgtgtctgcagctgtgggaggcacagtcaccatcaattgccaggccagtgagagtgttggtaataacaactacttatcctggtatcagcaaagaccagggcagcctcccaaggtcttgatctacgatgcatccaggctggcatctggggtctcatcgcggttcaaaggcagtggatctgggacacagttcactctcaccatcagtggtgtgcaatgtgacgatgctgccacttactattgtctaggcggttatgttagtagtggttggtatggggctttcggcggagggaccgaggtggtggtcaaa

SEQ ID NO: 271 - protein, 113 aa, Oryctolagus cuniculus
QSLEESGGDLVKPGASLILTCTASGFDFSSNALCWVRQAPGKGLEWIASIYAGGDTYYATWAKGRFTVSKTSSTTVFLQMTSLTAADTATYFCARGAMTYSLWGPGTLVTVSS

SEQ ID NO: 272 - DNA, 339 bp, Oryctolagus cuniculus
cagtcgttggaggagtccgggggagacctggtcaagcctggggcatccctgatactcacctgcacagcctctggattcgacttcagtagcaatgcactgtgctgggtccgccaggctccagggaaggggctggagtggatcgcatccatttatgctggtggtgacacttactacgcgacctgggcgaaaggccgattcaccgtctccaaaacctcgtcgaccacggtgtttctgcagatgaccagtctgacagccgcggacacggccacctatttctgtgcgaggggtgctatgacttatagtttgtggggcccaggcaccctggtcaccgtctcctca

SEQ ID NO: 273 - protein, 115 aa, Oryctolagus cuniculus
ADIVMTQTPASVEAAMGGTVTINCQASQSVHNNNYLSWYQQKPGQPPKLLIYQASKLASGGPSRFKGSGSGTEFTLTISDLECADAATYYCQSYYYSGSSGAVNSFGGGTEVVVK

SEQ ID NO: 274 - DNA, 345 bp, Oryctolagus cuniculus
gctgacattgtgatgacccagactccagcctccgtggaggcagctatgggaggcacagtcaccatcaactgccaggccagtcagagtgttcataataataactacttatcctggtatcagcagaaaccagggcagcctcccaagctcctgatctaccaggcatccaaactggcatctgggggcccatcgcggttcaaaggcagtggatctgggacagagttcactctcaccatcagcgacctggagtgtgccgatgctgccacttactactgtcaaagctattattatagtggtagtagtggtgccgttaattctttcggcggagggaccgaggtggtggtcaaa

SEQ ID NO: 275 - protein, 113 aa, Oryctolagus cuniculus
QEQLKESGGDLVTPGTPLTLTCTVSGFSLSSNAISWVRQAPGKGLEWIGVIYAGGGAFYANWAKGRFTFSKTSTTVDLKMTSLTTEDTASYFCTRGYTYLAFWGQGTLVTVSS

SEQ ID NO: 276 - DNA, 339 bp, Oryctolagus cuniculus
caggagcagttgaaggagtccgggggagacctggtcacgcctgggacacccctgacactcacctgcacagtctctggattctccctcagtagcaatgcaataagctgggtccgccaggctccagggaaggggctggaatggatcggagtcatttatgctggtggtggcgcattctacgcgaactgggcgaaaggccgattcaccttctccaaaacctcgaccacggtggatctgaaaatgaccagtctgacaaccgaggacacggcctcttatttctgtaccagaggctatacttatttggccttctggggccagggcaccctggtcaccgtctcctca

SEQ ID NO: 277 - protein, 111 aa, Oryctolagus cuniculus
ADIVMTQTPASVEAAVGGTVTINCQASQSISNLISWYQQKPGQPPKLLIYKASTLASGVSSRFKGSGSGTEYTLTISDLECADAATYYCQGSAYGTSDVCAFGGGTEVVVK

SEQ ID NO: 278 - DNA, 333 bp, Oryctolagus cuniculus
gctgacattgtgatgacccagactccagcctccgtggaggcagctgtgggaggcacagtcaccatcaattgccaggccagtcagagcattagcaacctcatttcttggtatcagcagaaaccagggcagcctcccaaactcctgatctacaaggcatccactctggcatctggggtctcatcgcggttcaaaggcagtggatctgggacagagtacactctcaccatcagcgacctggagtgtgccgatgctgccacttactactgtcaaggctctgcttatggtactagtgatgtttgtgctttcggcggagggaccgaggtggtggtcaaa

SEQ ID NO: 279 - protein, 112 aa, Oryctolagus cuniculus
QSVEESGGRLVTPGTPLTLTCTVSGIDLSSNAMTWVRQAPGEGLEWIGAIDANGSPYYTNWAKGRFTISKTSTTVTLKMTSPTTEDTATYFCARGYTRLDLWGQGTLVTVSS

SEQ ID NO: 280 - DNA, 336 bp, Oryctolagus cuniculus
cagtcggtggaggagtccgggggtcgcctggtcacgcctgggacacccctgacactcacctgcacagtctctggaatcgacctcagtagcaatgcaatgacctgggtccgccaggctccaggggaggggctggaatggatcggagccattgatgctaatggtagcccatactacacgaactgggcgaaaggccgattcaccatctccaaaacctcgaccacggtgactctgaaaatgaccagtccgacaaccgaggacacggccacctatttctgtgccagagggtatactcggttggatctctggggccagggcaccctggtcaccgtctcctca

SEQ ID NO: 281 - protein, 111 aa, Oryctolagus cuniculus
QVLTQTPSSVSAAVGGTVTINCQSSQSILSDNYLAWYQQKPGQPPKLLIYQASKLVSGVSSRFKGSGSGTGFTLTISGVQCDDAATYYCQGAYDSSDWYGAFGGGTEVVVK

SEQ ID NO: 282 - DNA, 333 bp, Oryctolagus cuniculus
caagtgctgacccagactccatcctccgtgtctgcagctgtgggaggcacagtcaccatcaattgccagtccagtcagagtattttgagtgacaactacttagcctggtatcagcagaaaccagggcagcctcccaagctcctgatctaccaggcatccaaattggtttctggggtctcatcgcgattcaaaggcagtggatctgggacaggattcactctcaccatcagcggcgtgcagtgtgacgatgctgccacttactactgtcaaggcgcttatgatagtagtgattggtacggtgctttcggcggagggaccgaggtggtggtcaaa

SEQ ID NO: 283 - protein, 112 aa, Oryctolagus cuniculus
QSVEESGGRLVTPGTPLTLTCTVSGFSLNNYAMIWVRQAPGEGLEYIGFINSGGSAYYASWAKGRFTISRTSTTVDLKMTSLTAADTATYFCARGVPKMDLWGQGTLVTVSS

SEQ ID NO: 284 - DNA, 336 bp, Oryctolagus cuniculus
cagtcggtggaggagtccgggggtcgcctggtcacgcctgggacacccctgacactcacctgcaccgtctctggattctccctcaataactatgcaatgatctgggtccgccaggctccaggggaggggctggaatacatcggattcattaattctggtggtagcgcatactacgcgagctgggcaaaaggccgattcaccatctccagaacctcgaccacggtggatctgaaaatgaccagtctgacagccgcggacacggccacctatttctgtgccagaggggttcctaagatggacttgtggggccaaggcaccctggtcaccgtctcctca

SEQ ID NO: 285 - protein, 111 aa, Oryctolagus cuniculus
IVMTQTPSSVSAAVGGTVTINCQASQSVYNGNELSWYQQKPGQPPKLLIYAASILASGVPSRFKGSGWGTHFTLTISDVVCDDAATYYCAGYQSSVIDDIGFGGGTEVVVK

SEQ ID NO: 286 - DNA, 333 bp, Oryctolagus cuniculus
atcgtgatgacccagactccatcctccgtgtctgcagctgtgggaggcacagtcaccatcaattgccaggccagtcaaagtgtttataatggcaacgaattatcctggtatcagcagaaaccagggcagcctcccaagctcctgatctatgctgcatccattttggcatccggggtcccatcgcggttcaaaggcagtgggtgggggacacacttcactctcaccatcagcgatgtggtgtgcgacgatgctgccacttactactgtgcaggatatcaaagtagcgttattgatgatattggtttcggcggagggaccgaggtggtggtcaaa

SEQ ID NO: 287 - protein, 113 aa, Oryctolagus cuniculus
QSVEESGGRLVTPGGSLTLTCTVSGFSLNNYAMIWVRQAPGEGLEYIGFINTGDRAYYASWAKGRFTISKTSSTTVDLKMTSLTAADTATYFCARGVPAMGLWGQGTLVTVSS

SEQ ID NO: 288 - DNA, 339 bp, Oryctolagus cuniculus
cagtcggtggaggagtccgggggtcgcctggtaacgcctggaggatccctgacactcacctgcacagtctctggattctccctcaataactatgcaatgatctgggtccgccaggctccaggggagggactggaatacatcggattcattaatactggtgatcgcgcatactatgcgagctgggcaaaaggccgattcaccatctccaaaacctcgtcgaccacggtggatctgaaaatgaccagtctgacagccgcggacacggccacctatttctgtgccagaggggttcctgctatgggcttgtggggccagggcaccctggtcaccgtctcctca

SEQ ID NO: 289 - protein, 111 aa, Oryctolagus cuniculus
IVMTQTPSPVSAAVGDPVTINCQASQSVYNNNELSWYQQKPGQAPKLLIYAASYVASGVPSRFKGSGSGTQFTLTISNVVCDDAATYYCAGYESSGIDDIGFGGGTEVVVK

SEQ ID NO: 290 - DNA, 333 bp, Oryctolagus cuniculus
atcgtgatgacccagactccatctcccgtgtctgcagctgtgggagatccagtcaccatcaattgccaggccagtcagagtgtttataataataacgaattatcctggtatcagcagaaacctgggcaggctcccaagctcctgatctatgctgcatcctatgtggcatctggggtcccatcgcggttcaaaggcagtggatctgggacgcagttcactctcaccatcagcaatgtggtgtgtgacgatgctgccacttactactgtgcaggatatgaaagtagtggtattgatgatattggtttcggcggagggaccgaggtggtggtcaaa

SEQ ID NO: 291 - protein, 113 aa, Oryctolagus cuniculus
QSVEESGGRLVTPGTPLTLTCTVSGFSLSSNAMTWVRQAPGQGLEWIGIIYASGSTYYASWAKGRFTISKTSSTTVDLKMTSPTTEDTATYFCARGFARLPLWGQGTLVTVSS

SEQ ID NO: 292 - DNA, 339 bp, Oryctolagus cuniculus
cagtcggtggaggagtccgggggtcgcctggtcacgcctgggacacccctgacactcacctgcacagtctctggattctccctcagtagcaatgcaatgacctgggtccgccaggctccagggcaggggctggaatggatcggaatcatttatgctagtggtagcacatactacgcgagctgggcgaaaggccgattcaccatctccaaaacctcgtcgaccacggtggatctgaaaatgaccagtccgacaaccgaggacacggccacctatttctgtgccagaggatttgcccggttgccgttgtggggccagggcaccctggtcaccgtctcctca

SEQ ID NO: 293 - protein, 110 aa, Oryctolagus cuniculus
QVLTQTPSSVSAAVGGTVTINCQSSQSVGSNYLSWYQQKPGQPPKLLIYDASTLASGVPSRFSGSGSGTQFTLTISGVQCDDAATYYCQGSYYSSDWYGAFGGGTEVVVK

SEQ ID NO: 294 - DNA, 330 bp, Oryctolagus cuniculus
caagtgctgacccagactccatcctccgtgtctgcagccgtgggaggcacagtcaccatcaattgccagtccagtcagagtgttggtagtaactacttatcctggtatcagcagaaaccagggcagcctcccaagctcttgatctacgatgcatccactctggcatctggggtcccatcgcggtttagcggcagtggatctgggacacagttcactctcaccatcagcggcgtgcagtgtgacgatgctgccacttactactgtcaaggcagttattatagtagtgattggtacggtgctttcggcggagggaccgaggtggtggtcaaa

SEQ ID NO: 295 - protein, 117 aa, Oryctolagus cuniculus
QSLEESGGDLVKPGASLTLTCTATGFSFNTNYYMCWVRQAPGKGLELIACSYTTSGSTYYATWAKGRFTFSKTSSTTVTLQMTSLTAADTATYFCVKYGAGYTYNLWGPGTLVTVSS

SEQ ID NO: 296 - DNA, 351 bp, Oryctolagus cuniculus
cagtcgttggaggagtccgggggagacctggtcaagccgggggcatccctgacactcacctgcacagccactggattctccttcaataccaactactacatgtgctgggtccgccaggctccagggaaggggctggagttgatcgcatgcagttatactactagtggtagtacttactatgcgacctgggcgaaaggccgattcaccttctccaaaacctcgtcgaccacggtgactctgcaaatgaccagtctgacagccgcggacacggccacctatttctgtgtgaaatatggtgctggttatacttataacttgtggggcccaggcaccctggtcaccgtctcctca

SEQ ID NO: 297 - protein, 110 aa, Oryctolagus cuniculus
ALVMTQTPSSVSAAVGGTVTIKCQASESISNYLAWYQQKPGQPPNLLIYRASTLESGVPSRFKGSGSGTEFTLTISDLECADAATYFCQQGYSNTNLDNIFGGGTEVVVK

SEQ ID NO: 298 - DNA, 330 bp, Oryctolagus cuniculus
gcccttgtgatgacccagactccatcctccgtgtctgcagctgtgggaggcacagtcaccatcaagtgccaggccagtgagagcattagtaactacttagcctggtatcagcagaaaccagggcagcctcccaatctcctgatctacagggcatccactctggaatctggggtcccatcgcggttcaaaggcagtggatctgggacagagttcactctcaccatcagcgacctggagtgtgccgatgctgccacttacttctgtcagcagggttacagtaatactaatcttgataatattttcggcggagggaccgaggtggtggtcaaa

SEQ ID NO: 299 - protein, 113 aa, Oryctolagus cuniculus
QSVEESGGRLVTPGTPLTLTCTVSGFSLSSNAISWVRQAPGEGLEYIGWIDATGSAYYATWAKGRFTISKTSSTTVDLKMTSPTTEDTATYFCARGFRYSAFWGQGTLVTVSS

SEQ ID NO: 300 - DNA, 339 bp, Oryctolagus cuniculus
cagtcggtggaggagtccgggggtcgcctggtcacgcctgggacacccctgacactcacctgcaccgtctctggattctccctcagtagcaatgcaataagctgggtccgccaggctccaggggaggggctggagtacatcggatggattgatgctactggtagcgcatactacgcgacctgggcgaaaggccgattcaccatctctaaaacctcgtcgaccacggtggatctgaagatgaccagtccgacaaccgaggacacggccacctatttctgtgccagagggtttaggtattctgcgttctggggccaaggcaccctggtcaccgtctcctca

SEQ ID NO: 301 - protein, 110 aa, Oryctolagus cuniculus
QVLTQTPSPVSVAVGGTVTINCQASQSVYNNNYLSWYQQKPGQPPKLLIYDTSTLASGIPSRFKGSGSGTQFTLTISDLECDDAATYYCAGTYSTSDWSVAFGGGTEVVV

SEQ ID NO: 302 - DNA, 333 bp, Oryctolagus cuniculus
caagtgctgacccagacaccatcgcccgtgtctgtagctgtgggaggcacagtcaccatcaattgccaggccagtcagagtgtttataataacaactacttatcctggtatcaacagaaaccagggcagcctcccaagctcctgatctatgatacatccactctggcatctgggatcccatcgcggttcaaaggcagtggatctgggacacagttcactctcaccatcagcgacctggagtgtgacgatgctgccacttattattgtgcaggcacttattctactagtgattggtctgttgctttcggcggagggaccgaggtggtggtcaaa

SEQ ID NO: 303 - protein, 116 aa, Oryctolagus cuniculus
QSVEESGGRLVTPGTPLTLTCTVSGIDLSTYFMSWVRQAPGKGLEYIGWINTNDKIYYASWAKGRFTISTTSTTVDLKITSPTTEDTATYFCGSPYPRYASGLNLWGQGTLVTVSS

SEQ ID NO: 304 - DNA, 348 bp, Oryctolagus cuniculus
cagtcggtggaggagtccgggggtcgcctggtcacgcctgggacacccctgacactcacctgcacagtctctggaatcgacctcagtacctatttcatgagctgggtccgccaggctccagggaaggggctggaatacatcgggtggattaatactaatgataaaatatactacgcgagctgggcgaagggccgattcaccatctccacaacctcgaccacggtggatctgaaaatcaccagtccgacaaccgaggacacggccacctatttctgtggcagtccttatcctaggtatgctagtggtcttaacttgtggggccaaggcaccctggtcaccgtctcctca

SEQ ID NO: 305 - protein, 111 aa, Oryctolagus cuniculus
ADVVMTQTPASVSEPVGGTVTIKCQASQSIHNYLAWYQQKPGQPPKLLIYSASNLASGVSSRFKGSGSGTEYTLTISDLECADAATYYCQCTYYGSSYENTFGGGTEVVVK

SEQ ID NO: 306 - DNA, 333 bp, Oryctolagus cuniculus
gccgatgttgtgatgacccagactccagcctccgtgtctgaacctgtgggaggcacagtcaccatcaagtgccaggccagtcagagcattcataattacttagcctggtatcagcagaaaccagggcagcctcccaagctcctgatctattctgcatccaatctggcatctggggtctcatcgcggttcaaaggcagtggatctgggacagaatacactctcaccatcagcgacctggagtgtgccgatgctgccacttactactgtcaatgtacttattatggtagtagttatgagaatactttcggcggagggaccgaggtggtggtcaaa

SEQ ID NO: 307 - protein, 109 aa, Oryctolagus cuniculus
QSLEESGGRLVTPGGSLTLTCTVSGIDLSRYHMSWVRQAPGKGLEWIATTHIDGGVYYAIWAKGRFTISKTSTTVDLKMTSLTAEDTATYFCARKFDLWGQGTLVTVSS

SEQ ID NO: 308 - DNA, 328 bp, Oryctolagus cuniculus
cagtcgctggaggagtccgggggtcgcctggtaacgcctggaggatccctgacactcacctgcacagtctctggaatcgacctcagtaggtaccacatgagctgggtccgccaggctccagggaaggggctggaatggatcgcaacgactcatattgatggtggcgtatactacgcgatttgggcgaaaggccgattcaccatctccaaaacctcgaccacggtggatctgaaaatgaccagtctgacagccgaggacacggccacctatttctgtgccagaaagtttgacttgtggggccaaggcaccctggtcaccgtctcctcag

SEQ ID NO: 309 - protein, 111 aa, Oryctolagus cuniculus
QVLTQTPSSVSAAVGGTVTISCQSSESVSNNNWLSWYQQKSGQPPKLLIYQASKLASGVSSRFKGSGSGTQFTLTISDLECADAATYYCQGGYYDSGWYYAFGGGTEVVVK

SEQ ID NO: 310 - DNA, 333 bp, Oryctolagus cuniculus
caagtgctgacccagactccatcgtccgtgtctgcagctgtgggaggcacagtcaccatcagttgccagtccagtgagagcgtttcaaataataactggttatcctggtatcagcagaaatcagggcagcctcccaagctcctgatctaccaggcatccaaactggcatctggggtctcatcgcggttcaaaggcagtggatctgggacacagttcactctcaccatcagcgacctggagtgtgccgatgctgccacttactactgtcaaggcggttattatgatagtggttggtactatgctttcggcggagggaccgaggtggtggtcaaa

SEQ ID NO: 311 - protein, 112 aa, Oryctolagus cuniculus
QSVEESGGRLVTPGTPLTLTCTVSGFSLSSNAISWVRQAPGKGLEWIGFIDSEGSASYASWANGRFTISKTSNTVDLKMTGLTTEDTATYFCARGFRYLPLWGQGTLVTVSS

SEQ ID NO: 312 - DNA, 336 bp, Oryctolagus cuniculus
cagtcggtggaggagtccgggggtcgcctggtcacgcctgggacacccctgacactcacctgcaccgtctctggattctccctcagtagcaatgcaataagctgggtccgccaggctccagggaaggggctggagtggatcggattcattgatagtgagggtagcgcatcctacgcgagctgggcgaatggtcgattcaccatctccaaaacctcgaacacggtggatctgaaaatgaccggtctgacaaccgaggacacggccacctatttctgtgccagaggatttcggtacttgcccttgtggggccaaggcaccctggtcaccgtctcctca

SEQ ID NO: 313 - protein, 112 aa, Oryctolagus cuniculus
AVLTQTPSPVSAAVGGTVTINCQASQSVYYNNYLAWYQQKPGQPPKLLIYDTSKLASGVPSRFKGSGSGTQFTLTISGVQCDDAASYFCQGTYYSSGWYWNAFGGGTEVVVK

SEQ ID NO: 314 - DNA, 336 bp, Oryctolagus cuniculus
gccgtgctgacccagacaccatcccccgtgtctgcagctgtgggaggcacagtcaccatcaattgccaggccagtcagagtgtttattataacaactacttagcctggtatcagcagaaaccagggcagcctcccaaactcctgatctacgatacatccaaattggcatctggggtcccatcccggttcaaaggcagtggatctgggacacagttcactctcaccatcagcggcgtgcagtgtgacgatgctgcctcttacttctgtcaaggcacttattatagtagtggttggtactggaatgctttcggcggagggaccgaggtggtggtcaaa

SEQ ID NO: 315 - protein, 113 aa, Oryctolagus cuniculus
QEQLKESGGDLVTPGTPLTLTCTVSGFSLSSNAISWVRQAPGKGLEWIGVIYAGGGAFYASWAKGRFTFSKTSTTVDLKMTSLTTEDTASYFCTRGYTYLAFWGQGTLVTVSS

SEQ ID NO: 316 - DNA, 339 bp, Oryctolagus cuniculus
caggagcaactgaaggagtccgggggagacctggtcacgcctgggacacccctgacactcacctgcacagtctctgggttctccctcagtagcaatgcaataagctgggtccgccaggctccagggaaggggctggaatggatcggagtcatttatgctggtggtggcgcattctacgcgagctgggcgaaaggccgattcaccttctccaaaacctcgaccacggtggatctgaaaatgaccagtctgacaaccgaggacacggcctcctatttctgtaccagaggctatacttatttggccttctggggccagggcaccctggtcaccgtctcctca

SEQ ID NO: 317 - protein, 110 aa, Oryctolagus cuniculus
DVVMTQTPASVSEPVGGTVTIRCQASQSINNFLSWYQQKPGQPPKLLIYKASTLASGVSSRFKGSGSGTEYTLTISDLECADAATYYCQGSAYGTSDVCAFGGGTEVVVK

SEQ ID NO: 318 - DNA, 330 bp, Oryctolagus cuniculus
gatgttgtgatgacccagactccagcctccgtgtctgaacctgtgggaggcacagtcaccatcaggtgccaggccagtcagagcattaacaatttcttatcttggtatcagcagaaaccagggcagcctcccaagctcctgatctacaaggcatccactctggcatctggggtctcatcgcggttcaaaggcagtggatctgggacagagtacactctcaccatcagcgacctggagtgtgccgatgctgccacttactactgtcaaggctctgcttatggtactagtgatgtttgtgctttcggcggagggaccgaggtggtggtcaaa

SEQ ID NO: 319 - protein, 113 aa, Oryctolagus cuniculus; identical to SEQ ID NO: 271

SEQ ID NO: 320 - DNA, 339 bp, Oryctolagus cuniculus; identical to SEQ ID NO: 272

SEQ ID NO: 321 - protein, 115 aa, Oryctolagus cuniculus
ADIVMTQTPASVEAAVGGTVTINCQASQSVHNNNYLSWYQQKPGQPPKLLIYQASKLASGGPSRFKGSGSGTEFTLTISDLECADAATYYCQSYYYSGCSGAVNSFGGGTEVVVK

SEQ ID NO: 322 - DNA, 345 bp, Oryctolagus cuniculus
gctgacattgtgatgacccagactccagcctccgtggaggcagctgtgggaggcacagtcaccatcaactgccaggccagtcagagtgttcataataataactacttatcctggtatcagcagaaaccagggcagcctcccaagctcctgatctaccaggcatccaaactggcatctgggggcccatcgcggttcaaaggcagtggatctgggacagagttcactctcaccatcagcgacctggagtgtgccgatgctgccacttactactgtcaaagctattattatagtggttgtagtggtgccgttaattctttcggcggagggaccgaggtggtggtcaaa

SEQ ID NO: 323 - protein, 110 aa, Oryctolagus cuniculus
QSLEESGGRLVTPGGSLTLTCTVSGIDLSSNAVGWVRQAPGKGLEYIGVISVSGNIYYANWARGRFTISKTSSTTVDLKMTSLTAADTATYFCARPWDLWGQGTLVTVSS

SEQ ID NO: 324 - DNA, 330 bp, Oryctolagus cuniculus
cagtcgctggaggagtccgggggtcgcctggtaacgcctggaggatccctgacactcacctgcacagtctctggaatcgacctcagtagcaatgcagtgggctgggtccgccaggctccagggaaggggctggaatacatcggagtcattagtgttagtggtaacatatactacgcgaactgggcgagaggccgattcaccatctccaaaacctcgtcgaccacggtggatctgaaaatgaccagtctgacagccgcggacacggccacctatttctgtgccagaccctgggacttgtggggccaaggcaccctggtcaccgtctcctca

SEQ ID NO: 325 - protein, 110 aa, Oryctolagus cuniculus
DVVMTQTPASVSEAVGGTVTIKCQASESISSWLAWYQQKPGQPPKLLIYLASTLASGVPSRFKGSGSGTQFTLTISDLECADAATYYCQCSSYTSGYVAAFGGGTEVVVK

SEQ ID NO: 326 - DNA, 330 bp, Oryctolagus cuniculus
gatgttgtgatgacccagactccagcctccgtgtctgaagctgtgggaggcacagtcaccatcaagtgccaggccagtgagagcattagcagttggttagcctggtatcagcagaaaccagggcagcctcccaagctcctgatctatctggcatctactctggcatctggggtcccatcgcggttcaaaggcagtggatctgggacacagttcactctcaccatcagcgacctggagtgtgccgatgctgccacttactactgtcaatgttcttcttatacgagtggttatgttgccgctttcggcggagggaccgaggtggtggtcaaa

SEQ ID NO: 327 - protein, 116 aa, Oryctolagus cuniculus
QSMEESGGRLVTPGTPLTLTCTVSTFSLNSFHMSWVRQAPGKGLEWIGVIHPNDATYYASWAKGRFTISKTSTTVDLKITSPTTEDTATYFCARDLAGYSTGGSFWGQGTLVTVSS

SEQ ID NO: 328 - DNA, 348 bp, Oryctolagus cuniculus
cagtcaatggaggagtccgggggtcgcctggtcacgcctgggacacccctgacactcacctgcacagtctctacattctccctcaatagtttccacatgagctgggtccgccaggctccagggaaggggctggaatggatcggcgtcattcatcctaatgatgccacatactacgcgagctgggcgaaaggccgattcaccatctccaaaacctcgaccacggtggatctgaaaatcaccagtccgacaaccgaggacacggccacctatttctgtgccagagatcttgctggttatagtactggtggtagcttctggggccaaggcaccctggtcaccgtctcctca

SEQ ID NO: 329 - protein, 112 aa, Oryctolagus cuniculus
ALVLTQTPSPVSAAVGGTVTVSCQASQSVYNNNWLSWFQQKPGQPPKLLIYRASTLASGVPSRFSGSGSGTQFTLTISGVQCADAATYYCAAYKSWSNDDFGFGGGTEVVVK

SEQ ID NO: 330 - DNA, 336 bp, Oryctolagus cuniculus
gcgcttgtgctgacccagactccatctcccgtgtctgcagctgtgggaggcacagtcaccgtcagttgccaggccagtcagagtgtttataataacaactggttatcctggtttcagcagaaaccagggcagcctcccaagctcctgatctacagggcatccactctggcatctggggtcccatcacggttcagcggcagtggatctgggacacagttcactctcaccatcagtggcgtgcagtgtgccgatgctgccacttactactgtgcagcgtataaaagttggagtaatgatgattttggtttcggcggagggaccgaggtagtagtcaaa

SEQ ID NO: 331 - protein, 109 aa, Oryctolagus cuniculus
QSLEESGGRLVTPGTPLTITCTVSGIDLSSVAMGWVRQAPGKGLEYIGVISTSGNKYYATWAKGRFTISKTSTTVELKVTSPTTEDTATYFCARAWNLWGQGTLVTVSS

SEQ ID NO: 332 - DNA, 327 bp, Oryctolagus cuniculus
cagtcgctggaggagtccgggggtcgcctggtcacgcctgggacacccctgacaatcacctgcacggtctctggaatcgacctcagtagcgttgcaatgggctgggtccgccaggctccagggaaggggctggaatacatcggagtcattagtactagtggtaataaatactacgcgacctgggcgaaaggccgattcaccatctccaaaacctcgaccacggtggagctgaaggtcaccagtccgacaaccgaggacacggccacctatttctgtgccagagcctggaacttgtggggccaaggcaccctggtcaccgtctcctca

SEQ ID NO: 333 - protein, 111 aa, Oryctolagus cuniculus
DVVMTQTPASVSEPVGGTVTIKCRASEDIESYLAWYRQKPGQPPKLLIYRASKLASGVPSRFSGSGSGTEYTLTISDLECADAATYYCQCTTYTSTYVGGGFGGGTEVVVK

SEQ ID NO: 334 - DNA, 333 bp, Oryctolagus cuniculus
gatgtagtgatgacccagactccagcctccgtgtctgaacctgtgggaggcacagtcaccatcaagtgccgggccagtgaggacattgaaagctatttagcctggtatcggcagaaaccagggcagcctcccaagctcctgatctacagggcatccaaactggcatctggggtcccatcgcggttcagtggcagtggatctgggacagagtacactctcaccatcagcgacctggagtgtgccgatgctgccacttactactgtcaatgtactacttatacgagtacttatgttggtggtggtttcggcggagggaccgaggtggtggtcaaa

SEQ ID NO: 335 - protein, 112 aa, Oryctolagus cuniculus
QSLEGSGGRLVKPDETLTITCTVSGFSLSSNAMSWVRQAPGKGLEWIGFIDAGGSAYYATWVNGRFTISKTSTTVDLKMTSLTTEDTATYFCAKGLSWSDFWGQGTLVTVSS

SEQ ID NO: 336 - DNA, 336 bp, Oryctolagus cuniculus
cagtcgctggaggggtccgggggtcgcctggtcaagcctgacgaaaccctgacaatcacctgcacagtctctggattctccctcagtagcaatgcaatgagctgggtccgccaggctccagggaaggggctggaatggatcggattcatagatgcgggtggtagcgcatactacgcgacctgggtgaatggccgattcaccatctccaaaacctcgaccacggtggatctgaaaatgaccagtctgacaaccgaggacacggccacctatttctgtgccaaaggactaagctggtctgacttttggggccagggcaccctggtcaccgtctcctca

SEQ ID NO: 337 - protein, 111 aa, Oryctolagus cuniculus
QVLTQTPSSVSAAVGGTVTVSCQSSQSVVSNNRLSWYQQKSGQPPKLLIYLASTLPSGVPSRFRGSGSGTQFTLTISDLGCDDAATYYCQGTYGSSSYYGAFGGGTEVVVK

SEQ ID NO: 338 - DNA, 333 bp, Oryctolagus cuniculus
caagtgctgacccagactccatcgtccgtgtctgcagctgtgggaggcacagtcaccgtcagttgccagtccagtcagagtgttgttagtaacaaccgcttatcctggtatcagcagaaatcagggcagcctcccaagctcctgatctatctggcatccactctgccatctggggtcccatcgcggttcaggggcagtggatctgggacacagttcactctcaccatcagcgacctgggctgtgacgatgctgccacttactactgtcaaggcacttatggtagtagtagttattacggagctttcggcggagggaccgaggtggtggtcaaa

SEQ ID NO: 339 - protein, 116 aa, Oryctolagus cuniculus
QSLEESGGRLVTPGTPLTLTCTASEFTISSFHMSWVRQAPGKGLEWIGVIHPNDITYYASWAKGRFTISKTSTTVELKITSPTTEDTATYFCVRDLTGGTTGGRLWGPGTLVTVSS

SEQ ID NO: 340 - DNA, 348 bp, Oryctolagus cuniculus
cagtcgctggaggagtccgggggtcgcctagtcacgcctgggacacccctgacactcacctgcacagcctctgaatttaccattagtagcttccacatgagctgggtccgccaggctccagggaaggggctggaatggatcggagtcattcatcccaatgatatcacatattacgcgagctgggcgaaaggccgattcaccatctccaaaacctcgaccacggtggagctgaagatcaccagtccgacaaccgaggacacggccacctatttctgtgtcagagatcttactggtggtactactggtggtaggttgtggggcccaggcaccctggtcaccgtctcctca

SEQ ID NO: 341 - protein, 112 aa, Oryctolagus cuniculus
ALVLTQTPSPVSAAVGDTVTVSCQSSKSVCNNDWLSWFQQKPGQPPKLLIYRASTLASGVPSRFKGSGSGTQFTLTISGVECDDAATYYCAGYASWNNDDFGFGGETEVVVK

SEQ ID NO: 342 - DNA, 336 bp, Oryctolagus cuniculus
gcccttgtgctgacccagactccatcccccgtgtctgcagctgtgggagacacagtcaccgtcagttgccagtccagtaagagtgtttgtaataacgactggttatcctggtttcagcagaaaccagggcagcctcccaagctcctgatctacagggcatccactctggcatctggggtcccatctcgattcaaaggcagtggatctgggacacaattcactctcaccatcagcggcgtggaatgtgacgatgctgccacttactactgtgcaggctatgcaagttggaataatgatgattttggtttcggcggagagaccgaggtggtggtcaaa

SEQ ID NO: 343 - protein, 110 aa, Oryctolagus cuniculus
QSVEESGGRLVTPGTSLTLTCTASGFSLSSYWMGWVRQAPEKGLEYIGIISTSENIYYATWAKGRFTISKTSSTTVDLKITSPTTEDTATYFCARWSDLWGQGTLVTVSS

SEQ ID NO: 344 - DNA, 330 bp, Oryctolagus cuniculus
cagtcggtggaggagtccgggggtcgcctggtcacgcctgggacatccctgacactcacctgcacagcctccggattctccctcagtagctactggatgggctgggtccgccaggctccagagaaggggctggaatacatcggaatcattagtacgagtgagaacatatactacgcgacctgggcgaaaggccgattcaccatctccaaaacctcgtcgaccacagtggatctgaaaatcaccagtccgacaaccgaggacacggccacctatttctgtgccagatggagtgacttgtggggccaaggcaccctggtcaccgtctcctca

SEQ ID NO: 345 - protein, 112 aa, Oryctolagus cuniculus
QVLTQTPASVSAAVGGTVTINCQSSQSVGSGNILSWYQQKPGQPPKLLIYQASKLASGVSSRFKGSGSGTQFTLIISDVQCDDGASYYCLGSYGCSSADCAAFGGGTEVVVK

SEQ ID NO: 346 - DNA, 336 bp, Oryctolagus cuniculus
caagtgctgacccagactccagcctccgtgtctgcagctgtgggaggcacagtcaccatcaactgccagtccagtcagagtgttggtagtggcaatatcttatcctggtatcagcagaaaccagggcagcctcccaagctcctgatctaccaggcatccaaactggcatctggggtctcatcgcggttcaaaggcagtggatctgggacacagttcactctcatcatcagcgacgtgcagtgtgacgatggtgcctcttactactgtctaggcagttatggttgtagtagtgctgattgtgctgctttcggcggagggaccgaggtggtggtcaaa

SEQ ID NO: 347 - protein, 112 aa, Oryctolagus cuniculus
QSVEVSGGRLVTPGTPLTLTCTVSGFSLSSNAISWVRQAPGKGLEWIGIIDSNGSTYYASWAKGRFTISKASTTVDLKITGPTTEDTATYFCGRGAIYPALWGQGTLVTVSS

SEQ ID NO: 348 - DNA, 336 bp, Oryctolagus cuniculus
cagtcggtggaggtgtccgggggtcgcctggtcacgcctgggacacccctgacactcacctgcacagtctctggattctccctcagtagcaatgcaataagctgggtccgccaggctccagggaaggggctggaatggatcggaatcattgatagtaatggtagcacatactacgcgagctgggcgaaaggccgattcaccatctccaaagcctcgaccacggtggatctgaaaatcaccggtccgacaaccgaggacacggccacctatttctgtggcagaggggcgatttatccggctttgtggggccaaggcaccctggtcaccgtctcctca

SEQ ID NO: 349 - protein, 112 aa, Oryctolagus cuniculus
AFELTQTPASVEAAVGGTVTIKCQASQSISSSYLSWYQQKPGQPPKLLIYKASTLASGVPSRFKGSGSGTQFTLTISGVQCDDAATYYCLYAYFGGSTAEHTFGGGTEVVVK

SEQ ID NO: 350 - DNA, 336 bp, Oryctolagus cuniculus
gcattcgaattgacccagactccagcctccgtggaggcagctgtgggaggcacagttaccatcaagtgccaggccagtcagagtattagtagtagctacttatcctggtatcagcaaaaaccagggcagcctcccaagctcctgatctacaaggcttccactctggcatctggggtcccatcgcggttcaaaggcagtggatctgggacacagttcactctcaccatcagtggcgtgcagtgtgacgatgctgccacttactactgtctatacgcttattttggtggtagtactgctgagcatactttcggcggagggaccgaggtggtggtcaaa

SEQ ID NO: 351 - protein, 109 aa, Oryctolagus cuniculus
QSLEESGGRLVTPGTPLTLTCTVSGIDLSRYYMSWVRQAPGKGLEWIATTHIDGGVYYANWAKGRFTISKTATTVDLKMTSLTAEDTATYFCARKFDLWGQGTLVTVSS

SEQ ID NO: 352 - DNA, 327 bp, Oryctolagus cuniculus
cagtcgctggaggagtccgggggtcgcctggtcacgcctgggacacccctgacactcacctgcacagtctctggaatcgacctcagtaggtactacatgagctgggtccgccaggctccagggaaggggctggaatggatcgcaacgactcatattgatggtggcgtatattacgcgaactgggcgaaaggccgattcaccatctccaaaaccgcgaccacggtggatctgaaaatgaccagtctgacagccgaggacacggccacctatttctgtgccagaaaatttgacttgtggggccaaggcaccctggtcaccgtctcctca

SEQ ID NO: 353 - protein, 111 aa, Oryctolagus cuniculus
QELTQTPSSVSAAVGGTVTISCQSSESVSNNNWLSWYQQKPGQPPKLLIYAASKLASGVPSRFTGSGSGTQFTLTISDLECADAATYYCQGGYYDSGWYYAFGGGTEVVVK

SEQ ID NO: 354 - DNA, 301 bp, Oryctolagus cuniculus
tgcagctgtgggaggcacagtcaccatcagttgccagtccagtgagagcgtttcaaataacaactggttatcctggtaccagcagaaaccagggcagcctcccaagctcctgatctatgctgcatccaagctggcaagtggggtcccatcgcggttcaccggcagtgggtctgggacacagttcactctcaccatcagcgacctggagtgtgccgatgctgccacttactactgccaaggcggttattatgatagtggttggtactatgctttcggcggagggaccgaggtggtggtcaaa

SEQ ID NO: 355 - protein, 112 aa, Oryctolagus cuniculus
QSAEESGGRLGTPGTPLTLTCTVSGFSLSSNAINWVRQAPGKGLEWIGIIDAPGSTYYASWAKGRFTISKTSTTADLKITSPTTEDTATYFCARNYAYFALWGPGTLVTVSS

SEQ ID NO: 356 - DNA, 336 bp, Oryctolagus cuniculus
cagtcggcggaggagtccgggggtcgcctgggcacgcctgggacacccctgacactcacctgtacagtctctggattctccctcagtagcaatgcaataaactgggtccgccaggctccagggaaggggctggaatggatcggaatcattgatgctcctggtagcacatactacgcgagctgggcgaaaggccgattcaccatctccaaaacctcgaccacggcggatctgaaaatcaccagtccgacaaccgaggacacggccacctatttctgtgccagaaattatgcctactttgccttatggggcccaggcaccctggtcaccgtctcctca

SEQ ID NO: 357 - protein, 112 aa, Oryctolagus cuniculus
AFEMTQTPSSVSEPVGGTVTIKCQASESVGSNNRLSWYQQKPGQPPKLLIYEASKLPSGVPSRFRGSGSGTQFTLTISDIQREDAATYYCLGWHASTDDGWAFGAGTNVGIE

SEQ ID NO: 358 - DNA, 336 bp, Oryctolagus cuniculus
gcattcgagatgacccagactccatcctccgtgtctgaacctgtgggaggcacagtcaccatcaagtgccaggccagtgagagtgttggtagtaacaaccgcttatcctggtatcagcagaaaccagggcagcctcccaagctcctgatctatgaagcatccaaactgccatctggggtcccgtcgcggttcagaggcagtggatctgggacacagttcactctcaccatcagcgacattcagcgtgaggatgctgccacctactattgtctaggctggcatgctagtactgatgatggttgggcattcggagctggcaccaatgtgggaatcgaa

SEQ ID NO: 359 - protein, 112 aa, Oryctolagus cuniculus
QSVKESGGGLFKPTDTLTLTCTVSGFSLSSNAITWVRQAPGKGLEWIGIIWSGGDTDYATWAKGRFTISKTSTTVDLEITSPTTEDTATYFCVKGATYSALWGPGTLVTVSS

SEQ ID NO: 360 - DNA, 336 bp, Oryctolagus cuniculus
cagtcagtgaaggagtccgggggaggcctcttcaagccaacggataccctgacactcacctgcaccgtctctggattctccctcagtagcaatgcaataacttgggtccgccaggctccagggaaggggctggaatggatcggaatcatttggagtggtggtgacaccgactacgcgacctgggcgaaaggccgcttcaccatctccaaaacctcgaccacggtggatctggaaatcaccagtccgacaaccgaggacacggccacctatttctgtgtcaaaggggctacttatagtgccttgtggggcccaggcaccctggtcaccgtctcctca

SEQ ID NO: 361 - protein, 111 aa, Oryctolagus cuniculus
ALVMTQTPSSVEADVGGTVTIKCQASQSISSNYYAWYQQKPGQPPKLLIYKASTLASGVSSRFRGSGSGTEYTLTISDLECADAATYYCQGFDYGNSNVGAFGGGTEVVVK

SEQ ID NO: 362 - DNA, 333 bp, Oryctolagus cuniculus
gcccttgtgatgacccagactccatcctccgtggaggcagatgtgggaggcacagtcaccatcaagtgccaggccagtcagagtattagtagtaactactatgcctggtatcagcagaaaccagggcagcctcccaagctcctgatctacaaggcatccactctggcatctggggtctcatcgcggttcagaggcagtggatctgggacagagtatactctcaccatcagcgacctggagtgtgccgatgctgccacttactactgtcaaggctttgattatggtaatagtaatgttggtgctttcggcggagggaccgaggtggtggtcaaa

SEQ ID NO: 363 - protein, 112 aa, Oryctolagus cuniculus
QSLEESGGRLVTPGTPLTLTCTVSGIDLSSNAMSWVRQAPGKGLEWIGYIWSGGNTDYASWAKGRFTISKTSTTVDLKITSPTTEDTATYFCARGGSYFPFWGPGTLVTVSS

SEQ ID NO: 364 - DNA, 336 bp, Oryctolagus cuniculus
cagtcgctggaggagtccgggggtcgcctggtcacgcctgggacacccctgacactcacctgcacagtctctggaatcgacctcagtagcaatgcaatgagctgggtccgccaggctccagggaaggggctggaatggatcggatacatttggagtggtggtaatacagactacgcgagctgggcgaaaggccgattcaccatctccaaaacctcgaccacggtggatctgaaaatcaccagtccaacaaccgaggacacggccacctatttctgtgccaggggggggtcatactttcccttctggggcccaggcaccctggtcaccgtctcctca

SEQ ID NO: 365 - protein, 113 aa, Oryctolagus cuniculus
DPVMTQTPSSTSAAVGGTVTINCQSSQSVYIDRLAWYQQKPGQPPKLLIYQASKLPSGVPSRFSGSGSGKQSTLTISGVQCDDAATYYCAGFYDSGSGTYTLAFGGGTEVVVK

SEQ ID NO: 366 - DNA, 339 bp, Oryctolagus cuniculus
gaccctgtgatgacccagactccatcttccacgtctgcggctgtgggaggcacagtcaccatcaactgccagtccagtcagagtgtttatatcgaccgcttagcctggtatcagcagaaaccagggcagcctcccaagctcctgatctaccaggcatccaaactgccatctggggtcccatcgcggttcagcggcagtggatctgggaaacagtccactctcaccatcagtggcgtgcagtgtgacgatgctgccacttactactgtgcagggttttatgatagtggtagtggcacttatacattagctttcggcggagggaccgaggtggtggtcaaa

SEQ ID NO: 367 - protein, 113 aa, Oryctolagus cuniculus
QSVEESGGRLVTPGTPLTLTCTVSGFSLSSNAISWVRQAPGKGLEWIGVIDAGGSTYFASWAKGLFTISKTSSTTVDLQMTSLTTEDTATYFCARGWSRHDFWGPGTLVTVSS

SEQ ID NO: 368 - DNA, 339 bp, Oryctolagus cuniculus
cagtcggtggaggagtccgggggtcgcctggtcacgcctgggacacccctgacactcacctgcaccgtctctggattctccctcagtagcaatgcaataagctgggtccgccaggctccagggaaggggctggaatggatcggagtcattgatgctggtgggtccacatacttcgcgagctgggcgaaaggcctattcaccatctccaaaacctcgtcgaccacggtggatctgcaaatgaccagtctgacaaccgaggacacggccacctatttctgtgccagaggttggagtagacatgacttctggggcccaggcaccctggtcaccgtctcctca

SEQ ID NO: 369 - protein, 109 aa, Oryctolagus cuniculus
DVVMTQTPASVSEPVGGTVTIKCQASQSISNILAWYQQKPGQPPRLLIYSASTLASGVSSRFKASGSGTEFTLTISDLECADAATYYCQGYDSTVGVGAFGGGTEVVVK

SEQ ID NO: 370 - DNA, 327 bp, Oryctolagus cuniculus
gatgttgtgatgacccagactccagcctccgtgtctgaacctgtgggaggcacagtcaccatcaagtgccaggccagtcagagcattagcaatatattagcctggtatcagcagaaaccagggcagcctcccaggctcctgatctattctgcatccactctggcatctggggtctcatcgcggttcaaggccagtggatctgggacagagttcactctcaccatcagcgacctggagtgtgccgatgctgccacttactactgtcaagggtatgatagtactgttggtgtgggtgctttcggcggagggaccgaggtggtggtcaaa

SEQ ID NO: 371 - protein, 114 aa, Oryctolagus cuniculus
QSLEESGGRLVTPGTPLTLTCTASGFDLSRYHMNWVRQAPGKGLEWIGIIYVSDDSYYASWAKGRFTISKTSTAVDLKITSPTTEDTATYFCARVGSVWSSKLWGPGTLVTVSS

SEQ ID NO: 372 - DNA, 342 bp, Oryctolagus cuniculus
cagtcgctggaggagtccgggggtcgcctggtcacgcctgggacacccctgacactcacctgcacagcctctggattcgacttaagtaggtaccacatgaactgggtccgccaggctccagggaaggggctggaatggatcggaatcatttatgttagtgatgactcatactacgcgagctgggcgaaaggccgattcaccatctccaaaacctcgaccgcggtggatctgaaaatcaccagtccgacaaccgaggacacggccacctatttctgtgccagagttggtagtgtttggagcagtaagttgtggggcccaggcaccctggtcaccgtctcctca

SEQ ID NO: 373 - protein, 110 aa, Oryctolagus cuniculus
DVVMTQTPASVSEPVGGTVTIKCQASENIYNNLAWYQQKPGQPPKLLIYRASTLESGVPSRFKGSGSGTEFTLTISDLECADAATYYCQSTYFGGSYVFAFGGGTEVVVK

SEQ ID NO: 374 - DNA, 330 bp, Oryctolagus cuniculus
gatgttgtgatgacccagactccagcctccgtgtctgaacctgtgggaggcacagtcaccatcaagtgccaggccagtgagaacatttacaacaatttagcctggtatcagcagaaaccagggcagcctcccaagctcctgatctacagggcatccactctggaatctggggtcccatcgcggttcaaaggcagtggatctgggacagagttcactctcaccatcagcgacctggagtgcgccgatgctgccacttactactgtcaatctacttattttggtgggagttatgtttttgctttcggcggagggaccgaggtggtggtcaaa

SEQ ID NO: 375 - protein, 112 aa, Oryctolagus cuniculus
QSLEESGGRLVTPGTPLTLTCTVSGFSLSSNAISWVRQAPGKGLEWIGTIYATDSTSYASWAKGRFTISKTSTTVDLKMTSLTAADTATYFCALGASYSALWGPGTLVTVSS

SEQ ID NO: 376 - DNA, 336 bp, Oryctolagus cuniculus
cagtcgctggaggagtccgggggtcgcctggtcacgcctgggacacccctgacactcacctgcacagtctctggattctccctcagtagcaatgcaataagttgggtccgccaggctccagggaaggggctggaatggatcggaaccatttatgctactgatagcacgtcctacgcgagctgggcaaaaggccgattcaccatctccaaaacctcgaccacggtggatctgaaaatgaccagtctgacagccgcggacacggccacctatttctgtgccttaggtgctagttattctgctttgtggggcccaggcaccctggtcaccgtctcctca

SEQ ID NO: 377 - protein, 112 aa, Oryctolagus cuniculus
AIVMTQTPSSKSVAVGDTVTINCQASESVASNDRLAWYQQKPGQRPKLLIYQASTLASGVPSRFKGSGSGTEFTLTISNVVCDDAATYYCAGYKSSSTDGNAFGGGTEVVVK

SEQ ID NO: 378 - DNA, 333 bp, Oryctolagus cuniculus
atcgtgatgacccagactccatcttccaagtctgtcgctgtgggagacacagtcaccatcaattgccaggccagtgagagtgttgctagtaacgaccgcttagcctggtatcagcagaaaccagggcagcgtcccaaactcctaatctaccaggcatccactctggcatctggggtcccatcgcggttcaaaggcagtggatctgggacagagttcactctcaccatcagcaatgtggtgtgtgacgatgctgccacttactactgtgcaggatataaaagtagtagtactgatggtaatgctttcggcggagggaccgaggtggtggtcaaa

SEQ ID NO: 379 - protein, 114 aa, Oryctolagus cuniculus
QSVEESGGGLVTPGGTLTLTCTASGFSLSSYDMSWVRRAPGKGLEWIGVIATGGRRDYASWAKGRFTVSKTSTTVDLKMTSLTAADTATYFCARYSDSDGYALWGPGTLVTVSS

SEQ ID NO: 380 - DNA, 342 bp, Oryctolagus cuniculus
cagtcggtggaggagtccggaggaggcctggtaacgcctggaggaaccctgacactcacctgcacagcctctggattctccctcagcagctacgacatgagctgggtccgccgggctccagggaaggggctggaatggatcggagtcattgctactggtggtagaagggactacgcgagctgggcaaaaggctgattcaccgtctccaaaacctcgaccacggtggatctgaaaatgaccagtctgacagccgcggacacggccacctatttctgtgccagatatagtgatagtgatggttatgccttgtggggcccaggcaccctggtcaccgtctcctca

SEQ ID NO: 381 - protein, 110 aa, Oryctolagus cuniculus
AEVVMTQAPASVEAAVGGTVTIKCQASESIGSWLAWYQQKPGQPPKLLIYSASTLAFGVPSRFSGSGSGTQFTLTISDLECADAATYYCQSNYYSTSGHAFGGGTEVVVK

SEQ ID NO: 382 - DNA, 330 bp, Oryctolagus cuniculus
gccgaagtagtgatgacccaggctccagcctccgtggaggcagctgtgggaggcacagtcaccatcaagtgccaggccagtgagagcattggcagttggttagcctggtatcagcagaaaccagggcagcctcccaagctcctgatctattctgcgtccactctggcatttggggtcccgtcgcggttcagcggcagtggatctgggacacagttcactctcaccatcagcgacctggagtgtgccgatgcggccacttactactgtcaaagtaattattatagtactagtgggcatgctttcggcggagggaccgaggtggtggtcaaa

SEQ ID NO: 383 - protein, 114 aa, Oryctolagus cuniculus
QSVEESGGRLVTPGTPLTLTCKASGFSLSNYWMNWVRQAPGKGLEWIGTINYGGSTYYASWAKGRFTISKTSTTVDLKITSPTTEDTATYFCARDNGAYTFDSWGPGTLVTVSS

SEQ ID NO: 384 - DNA, 342 bp, Oryctolagus cuniculus
cagtcggtggaggagtccgggggtcgcctggtcacgcctgggacacccctgacactcacctgcaaagcctctggattctccctcagtaactactggatgaactgggtccgccaggctccagggaaggggctggaatggatcggaaccattaattatggtggtagcacatactacgcgagctgggcgaaaggccgattcaccatctccaaaacctcgaccacggtggatctgaaaatcaccagtccgacaaccgaggacacggccacctatttctgtgccagagataatggtgcttatacttttgattcctggggcccaggcaccctggtcaccgtctcctca

SEQ ID NO: 385 - protein, 112 aa, Oryctolagus cuniculus
IVMTQTPSSASEPVGGTVTIKCQASQSVYNNNYLSWYQQKPGQSPKQLIYAASTLASGVPSRFKGSGSGTQFTLTISDVQCDDAASYYCLGKSSCSYDDCRAFGGGTEVVVK

SEQ ID NO: 386 - DNA, 336 bp, Oryctolagus cuniculus
attgtgatgacccagactccatcctctgcgtctgaacctgtgggaggcacagtcaccatcaaatgccaggccagtcagagtgtttataataacaactacttatcctggtatcagcagaaaccagggcagtctcccaagcaactgatctatgctgcatccactctggcatctggggtcccatcgcggttcaaaggcagtggatctgggacacagttcactctcaccatcagcgacgtgcagtgtgacgatgctgccagttactactgtctaggcaaatctagttgtagttatgatgattgtagggctttcggcggagggaccgaggtggtggtcaaa

SEQ ID NO: 387 - protein, 122 aa, Oryctolagus cuniculus
QEQLVESGGGLVQPEGSLTLTCTASGFSFSYYSWACWVRQAPGKGLEWIACIDGGGSRATYYASWAKGRFTISTTSSTTVTLQMTSLTAADTATYFCSRSDYNGYISYFDLWGPGTLVTVSS

SEQ ID NO: 388 - DNA, 366 bp, Oryctolagus cuniculus
caggagcagctggtggagtccgggggaggcctggtccagcctgagggatccctgacactcacctgcacagcctctggattctcctttagttactattcttgggcgtgctgggtccgccaggctccagggaagggactggagtggatcgcatgcattgatggtggtggtagtcgcgccacttactacgcgagctgggcgaaaggccgattcaccatctccacaacctcgtcgaccacggtgactctgcaaatgaccagtctgacagccgcggacacggccacttatttctgttcgagatccgactataatggttatatctcctactttgacttgtggggccccggcaccctggtcaccgtctcctca

SEQ ID NO: 389 - protein, 108 aa, Oryctolagus cuniculus
AFELTQTPSSVEAAVGGTVTINCQASQSIRSDLAWYQQKPGQPPKLLIYKASTLASGVPSRFRGSGSGTEYTLTISDLECADAATYYCQSYYHSSSTAFGGGTEVVVK

SEQ ID NO: 390 - DNA, 324 bp, Oryctolagus cuniculus
gcattcgagttgacccagactccatcctccgtggaggcagctgtgggaggcacagtcaccatcaattgccaggccagtcagagcattcgtagcgacttagcctggtatcagcagaaaccagggcagcctcccaagctcctgatctataaggcatccactctggcatctggggtcccatcgcggttcagaggcagtggatctgggacagagtacactctcaccatcagcgacctggagtgtgccgatgctgccacttactactgtcaaagctattatcatagtagtagtactgctttcggcggagggaccgaggtggtggtcaaa

SEQ ID NO: 391 - protein, 112 aa, Oryctolagus cuniculus
QSPEESGGRLVTPGTPLTLTCTVSGFSLSSNAISWVRQAPGKGLEWIGYIDANTNAYYASWAKGRVTISQTSTTVDLRITSPTSEDTATYFCARGVTYYPMWGPGTLVTVSS

SEQ ID NO: 392 - DNA, 336 bp, Oryctolagus cuniculus
cagtcgccggaggagtccgggggtcgcctggtcacgcctgggacacccctgacactcacctgcaccgtctctggattctccctcagtagcaatgcaataagctgggtccgccaggctccagggaaggggctggagtggatcggatatatagatgcgaatactaacgcatactacgcgagttgggcaaaaggccgagtcaccatctcccaaacctcgaccacggtggatctgaggatcaccagtccgacaagcgaagacacggccacctatttctgtgccagaggtgttacttattatccaatgtggggcccaggcaccctggtcaccgtctcctca

SEQ ID NO: 393 - protein, 111 aa, Oryctolagus cuniculus
DVVMTQTPASVSEPVGGTVTIKCQASQSIASRYCSWYQQKPGQPPKLLIYKASTLASGVSSRFKGSGSETEFTLTISDLECADAATYYCQGGYYGDSYVGAFGGGTEVVVK

SEQ ID NO: 394 - DNA, 333 bp, Oryctolagus cuniculus
gatgttgtgatgacccagactccagcctccgtgtctgaacctgtgggaggcacagtcaccatcaagtgccaggccagtcagagtattgctagtaggtactgctcctggtatcagcagaaaccagggcagcctcccaagctcctgatctacaaggcatccactctagcatctggggtctcatcgcggttcaaaggcagtggatctgagacagagttcactctcaccatcagcgacctggagtgtgccgatgctgccacttactactgtcaaggcggttattatggtgatagttatgttggcgctttcggcggagggaccgaggtggtggtcaaa

SEQ ID NO: 395 - protein, 112 aa, Oryctolagus cuniculus
QSVEESGGRLVKPDETLTLTCTVSGFSLSSNAISWVRQAPGKGLEWIGAIYSDDNTYYANWAKGRFTISKTSTTVDLKMTSLTTEDTATYFCGRGASRFDFWGPGTLVTVSS

SEQ ID NO: 396 - DNA, 336 bp, Oryctolagus cuniculus
cagtcggtggaggagtccgggggtcgcctggtcaagcctgacgaaaccctgacactcacctgcaccgtctctggattctccctcagtagcaatgcaataagttgggtccgccaggctccagggaaggggctggaatggatcggagccatttatagtgatgataacacatactacgcgaactgggcgaaaggccgattcaccatctccaaaacctcgaccacggtggatctgaaaatgaccagtctgacaaccgaggacacggccacctatttctgtggcagaggtgcttctaggtttgacttctggggcccaggcaccctggtcaccgtctcctca

SEQ ID NO: 397 - protein, 111 aa, Oryctolagus cuniculus
DVVMTQTPASVSAAVGGTVTIKCQASQSINGNYLAWYQQKPGQPPKLLIYKASTLTSGVPSRFKGSGSGTQFTLTISDLECADGATYYCQYTDYGSTYVGAFGGGTEVVVK

SEQ ID NO: 398 - DNA, 333 bp, Oryctolagus cuniculus
gatgttgtgatgacccagactccagcctccgtgtctgcagctgtgggaggcacagtcaccatcaagtgccaggccagtcagagtattaatggtaactacttagcctggtatcagcagaaaccagggcagcctcccaagctcctaatctataaggcatccactctgacatctggggtcccatcgcggttcaaaggcagtggatctgggacacagttcactctcaccatcagcgacctggagtgtgccgatggtgccacttactactgtcaatatactgattatggtagtacttatgttggtgctttcggcggagggaccgaggtggtggtcaaa

SEQ ID NO: 399 - protein, 112 aa, Oryctolagus cuniculus
QSLEESGGRLVTPGTPLTLTCTVSGFSLSSNAISWVRQAPGKGLEWIGYIDANTNAYYASWAKGRVTISQTSTTVDLRITSPTSEDTATYFCARGVTYYPMWGPGTLVTVSS

SEQ ID NO: 400 - DNA, 336 bp, Oryctolagus cuniculus
caatcgctggaggagtccgggggtcgcctggtcacgcctgggacacccctgacgctcacctgcaccgtctctggattctccctcagtagcaatgcaataagctgggtccgccaggctccagggaaggggctggagtggatcggatatatagatgcgaatactaacgcatactacgcgagttgggcaaaaggccgagtcaccatctcccaaacctcgaccacggtggatctgaggatcaccagtccgacaagcgaagacacggccacctatttctgtgccagaggtgttacttattatccaatgtggggcccaggcaccctggtcaccgtctcctca

SEQ ID NO: 401 - protein, 111 aa, Oryctolagus cuniculus
DVVMTQTPASVSEPVGGTVTIKCQASQSIGSRYWSWYQQQPGQPPKLLIYKASTLASGVSSRFKGSGSETEFTLTISDLECADAATYYCQGGYYGDSYVGAFGGGTEVVVK

SEQ ID NO: 402 - DNA, 333 bp, Oryctolagus cuniculus
gatgttgtgatgacccagactccagcctccgtgtctgaacctgtgggaggcacagtcaccatcaagtgccaggccagtcagagtattggtagtaggtactggtcctggtatcagcagcaaccagggcagcctcccaagctcctgatctacaaggcatccactctggcatctggggtctcatcgcggttcaaaggcagtggatctgagacagagttcactctcaccatcagcgacctggagtgtgccgatgctgccacttactactgtcaaggcggttattatggtgatagttatgttggcgctttcggcggagggaccgaggtggtggtcaaa

SEQ ID NO: 403 - protein, 113 aa, Oryctolagus cuniculus
QSVEESGGRLVTPGTPLTITCTVSGMDLSSNAMTWVRQAPGKGLEWIGIIYASDSTYYASWAKGRFTISKTSSTTVDLKITSPTTEDTATYFCARGATYIPLWGPGTLVTVSS

SEQ ID NO: 404 - DNA, 339 bp, Oryctolagus cuniculus
cagtcggtggaggagtccgggggtcgcctggtcacgcctgggacacccctgacaatcacctgcacagtctctggaatggacctcagtagcaatgcaatgacctgggtccgccaggctccagggaaggggctggaatggattggaatcatttatgctagtgatagcacatactacgcgagctgggcgaaaggccgattcaccatctccaaaacctcgtcgaccacggtggatctaaaaatcaccagtccgacaaccgaggacacggccacctatttctgtgccagaggtgctacttacattcccttgtggggcccaggcaccctggtcaccgtctcctca

SEQ ID NO: 405 - protein, 111 aa, Oryctolagus cuniculus
DVVMTQTPASVSEPVGGTVTINCQASQSISSSYLAWYQQKPGQPPKLLIYKASTLASGVSSRFKGSGSGTEFTLTISDLECADAATYYCQCTDYGSSYVGTFGGGTEVVVK

SEQ ID NO: 406 - DNA, 333 bp, Oryctolagus cuniculus
gatgttgtgatgacccagactccagcctccgtgtctgaacctgtgggaggcacagtcaccatcaattgccaggccagtcagagtattagtagtagctacttagcctggtatcagcagaaaccagggcagcctcccaagctcctgatctacaaggcatccactctggcatctggggtctcatcgcggtttaaaggcagtggatctgggacagagttcactctcaccatcagcgacctggagtgtgccgatgctgccacttactactgtcaatgtactgattatggtagtagttatgttggtactttcggcggagggaccgaggtggtggtcaaa

SEQ ID NO: 407 - protein, 112 aa, Oryctolagus cuniculus
QSVEESGGRLVKPDETLTLTCTVSGIDLSSNTMSWVRQAPGKGLEWIGLIGPVSNTYYANWAKGRVTISKTSTTVDLKITSPTTEDTATYFCARGWFQYSFWGPGTLVTVSS

SEQ ID NO: 408 - DNA, 336 bp, Oryctolagus cuniculus
cagtcggtggaggagtccgggggtcgcctggtcaagcctgacgaaaccctgacactcacctgcacagtgtctggaatcgacctcagtagcaatacaatgagctgggtccgccaggctccagggaaggggctggaatggatcggactgattggtcctgtcagtaacacatactacgcgaactgggcgaaaggccgggtcaccatctccaaaacctcgaccacggtggatctgaaaatcaccagtccgacaaccgaggacacggccacctatttctgtgccagaggctggttccaatatagcttctggggcccaggcaccctggtcaccgtctcctca

SEQ ID NO: 409 - protein, 112 aa, Oryctolagus cuniculus
AEVLMTQTPSSVEAPVGGTVTINCQASQSIDSYLSWYQQKPGQPPKLLIYKASTLASGVSSRFKGSGSGTEFTLTISDLECADAATYYCQGGYYSSSNNYITFGGGTEVVVK

SEQ ID NO: 410 - DNA, 336 bp, Oryctolagus cuniculus
gccgaagtactgatgacccagactccatcctccgtggaggcacctgtgggaggcacagtcaccatcaactgccaggccagtcagagcattgatagctacttatcctggtatcagcagaaaccagggcagcctcccaagctcctgatctacaaggcatccactctggcatctggggtctcatcgcggttcaaaggcagtggatctgggacagagttcactctcaccatcagcgacctggagtgtgccgatgctgccacttactactgtcaaggcggttattatagtagtagtaataattatattactttcggcggagggaccgaggtggtggtcaaa

SEQ ID NO: 411 - protein, 115 aa, Artificial Sequence
  Synthetic
EVQLVESGGGLVQPGGSLRLSCAASGFSLSSNAISWVRQAPGKGLEWVGIIYASGDTYYASWAKGRFTISRDNSKNTLYLQMNSLRAEDTAVYYCARGYTTLYFWGQGTLVTVSS

SEQ ID NO: 412 - DNA, 345 bp, Artificial Sequence
  Synthetic
gaggtgcagctggtggagtccggaggaggactggtgcagccaggaggcagcctgaggctgtcctgtgcagcctccggcttctctctgagctccaacgccatctcttgggtgaggcaggcacctggcaagggactggagtgggtgggcatcatctacgcctccggcgacacctactatgcctcttgggccaagggccggttcaccatctctagagataacagcaagaatacactgtatctgcagatgaattccctgagggccgaggacacagccgtgtactattgcgcccgcggctacaccacactgtatttttggggccagggcaccctggtgacagtgtctagc

SEQ ID NO: 413 - protein, 109 aa, Artificial Sequence
  Synthetic
DIQMTQSPSSVSASVGDRVTITCQASESISTRLAWYQQKPGKAPKLLIYSASTLPSGVPSRFSGSGSGTDFTLTISSLQPEDFATYYCQGGYSSGAGTAFGGGTKVEIK

SEQ ID NO: 414 - DNA, 327 bp, Artificial Sequence
  Synthetic
gacatccagatgacacagagcccaagctccgtgagcgcctccgtgggcgatagggtgaccatcacatgtcaggcctctgagagcatctccaccaggctggcatggtaccagcagaagccaggcaaggcccctaagctgctgatctattctgccagcaccctgccatccggagtgccatctaggttctccggctctggcagcggcacagactttaccctgacaatctctagcctgcagcccgaggatttcgccacctactattgccagggaggatactcctctggagcaggaaccgcctttggcggaggcacaaaggtggagatcaag

SEQ ID NO: 415 - protein, 117 aa, Artificial Sequence
  Synthetic
EVQLVESGGGLVQPGGSLRLSCAASGFDFSRYHMSWVRQAPGKGLEWVGIIYVSDNTYYATWAKGRFTISRDNSKNTLYLQMNSLRAEDTAVYYCVRVGSFWSSKLWGQGTLVTVSS

SEQ ID NO: 416 - DNA, 351 bp, Artificial Sequence
  Synthetic
gaggtgcagctggtggagagcggaggaggactggtgcagccaggaggctccctgcggctgtcttgcgccgccagcggcttcgatttttccaggtaccacatgtcctgggtgcgccaggcacctggcaagggactggagtgggtgggcatcatctacgtgagcgacaacacctactatgccacatgggccaagggccggttcaccatctccagagataactctaagaatacactgtacctgcagatgaatagcctgagggcagaggacaccgccgtgtactattgcgtgcgggtgggctccttttggagctccaagctgtggggacagggcaccctggtgacagtgtctagc

SEQ ID NO: 417 - protein, 110 aa, Artificial Sequence
  Synthetic
DIQMTQSPSTLSASVGDRVTITCQASENIYSSLAWYQQKPGKAPKLLIYEASNLESGVPSRFSGSGSGTEFTLTISSLQPDDFATYYCQSTYFGNSYVFAFGGGTKVEIK

SEQ ID NO: 418 - DNA, 330 bp, Artificial Sequence
  Synthetic
gacatccagatgacccagtccccatctacactgagcgcctccgtgggcgatagggtgaccatcacatgtcaggccagcgagaacatctacagctccctggcctggtatcagcagaagcccggcaaggcccctaagctgctgatctacgaggcctctaatctggagagcggagtgccatcccggttctctggaagcggatccggaaccgagtttaccctgacaatctctagcctgcagcccgacgatttcgccacctactattgccagtctacatactttggcaacagctacgtgttcgcctttggcggcggcacaaaggtggagatcaag

(19) United States
(12) Patent Application Publication
(10) Pub. No.: US 2022/0089758 A1
(43) Pub. Date: Mar. 24, 2022

(54) NOVEL ANTI-CD40 ANTIBODIES

(71) Applicant: REVMAB BIOSCIENCES USA, INC., South San Francisco, CA (US)

(72) Inventors: Mu-En LIN, South San Francisco, CA (US); Qiu YU, Moraga, CA (US); Hui LI, South San Francisco, CA (US); Yaohuang KE, San Francisco, CA (US)

(21) Appl. No.: 17/424,889

(22) PCT Filed: Jan. 22, 2020
(86) PCT No.: PCT/US2020/014493

§ 371 Date: Jul. 22, 2021

Related U.S. Application Data
(60) Provisional application No. 62/795,027, filed on Jan. 22, 2019.

(51) Int. Cl. C07K 16/28

(52) U.S. Cl. CPC C07K 16/2878; C07K 2317/24; C07K 2317/565

(57) ABSTRACT

The present disclosure provides anti-CD40 antibodies or antigen-binding fragments thereof, isolated polynucleotides encoding the same, pharmaceutical compositions comprising the same, and the uses thereof.

### CROSS-REFERENCE TO RELATED APPLICATION

This application claims priority to U.S. 62/795,027, filed on Jan. 22, 2019, the disclosure of which is incorporated herein by reference.

### SEQUENCE LISTING

The sequence listing that is contained in the file named “074233-8001WO01-SL-20200121_ST25”, which is 174 KB (as measured in Microsoft Windows) and was created on Jan. 21, 2020, is filed herewith by electronic submission and is incorporated by reference herein.

### BACKGROUND

### 1. Field of the Invention

The present disclosure generally relates to novel anti-human CD40 antibodies.

### 2. Description of Related Art

CD40 is a 48 kDa type I integral membrane glycoprotein and a member of the tumor necrosis factor (TNF) receptor superfamily. CD40 is expressed on a variety of cell types, including antigen-presenting cells (APCs) such as normal and neoplastic B cells, dendritic cells (DC), monocytes and macrophages, and nonimmune cells, including epithelial cells (e.g. keratinocytes), fibroblasts (e.g. synoviocytes), smooth muscle cells and platelets. CD40 is also expressed on a wide range of tumor cells including all B-lymphomas, 30-70% of solid tumors, melanomas and carcinomas.

CD40 signaling on APCs results in enhanced survival as well as activation of APC. CD40-mediated APC activation is involved in a variety of immune responses, including secretion of cytokines (e.g., IL-1, IL-6, IL-8, IL-10, IL-12, TNF-α and MIP-1α), up-regulation of costimulatory molecules (e.g., ICAM-1, LFA-3, CD80 and CD86), and APC proliferation. CD40 regulates immune responses against infections, tumors and self-antigens. CD40 is overexpressed on a wide range of malignant cells. The roles of CD40 in tumor inhibition and stimulation of the immune system make CD40 an attractive target for an antibody-based immunotherapy (van Mierlo G J, den Boer A T, Medema J P, et al. Proc Natl Acad Sci USA. 2002; 99(8): 5561 5566; French R R, Chan H T, Tutt A L, Glennie M J. Nat Med. 1999; 5(5):548-553).

There is a significant need for novel anti-CD40 antibodies.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides novel monoclonal anti-CD40 antibodies, amino acid and nucleotide sequences thereof, and uses thereof.

In one aspect, the present disclosure provides an isolated anti-CD40 antibody or an antigen-binding fragment thereof, comprising:
a) a heavy chain CDR1 sequence selected from the group consisting of: SEQ ID NOs: 1, 7, 13, 19, 25, 31, 37, 43, 49, 55, 61, 67, 73, 79, 85, 91, 97, 103, 109, 115, 121, 127, 133, 139, 145, 151, 157, 163, 169, 175, 181, 187, 193, 199, 205, 211, 217, 223, 229, 235, and 241;
b) a heavy chain CDR2 sequence selected from the group consisting of: SEQ ID NOs: 3, 9, 15, 21, 27, 33, 39, 45, 51, 57, 63, 69, 75, 81, 87, 93, 99, 105, 111, 117, 123, 129, 135, 141, 147, 153, 159, 165, 171, 177, 183, 189, 195, 201, 207, 213, 219, 225, 231, 237, and 243;
c) a heavy chain CDR3 sequence selected from the group consisting of: SEQ ID NOs: 5, 11, 17, 23, 29, 35, 41, 47, 53, 59, 65, 71, 77, 83, 89, 95, 101, 107, 113, 119, 125, 131, 137, 143, 149, 155, 161, 167, 173, 179, 185, 191, 197, 203, 209, 215, 221, 227, 233, 239, and 245;
d) a light chain CDR1 sequence selected from the group consisting of: SEQ ID NOs: 2, 8, 14, 20, 26, 32, 38, 44, 50, 56, 62, 68, 74, 80, 86, 92, 98, 104, 110, 116, 122, 128, 134, 140, 146, 152, 158, 164, 170, 176, 182, 188, 194, 200, 206, 212, 218, 224, 230, 236, and 242;
e) a light chain CDR2 sequence selected from the group consisting of: SEQ ID NOs: 4, 10, 16, 22, 28, 34, 40, 46, 52, 58, 64, 70, 76, 82, 88, 94, 100, 106, 112, 118, 124, 130, 136, 142, 148, 154, 160, 166, 172, 178, 184, 190, 196, 202, 208, 214, 220, 226, 232, 238, and 244; and
f) a light chain CDR3 sequence selected from the group consisting of: SEQ ID NOs: 6, 12, 18, 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, 240, and 246.

In certain embodiments, the antibody or an antigen-binding fragment thereof comprises a heavy chain variable region selected from the group consisting of SEQ ID NOs: 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, and 415, and a homologous sequence thereof having at least 80% sequence identity yet retaining specific binding affinity to CD40.

In certain embodiments, the antibody or an antigen-binding fragment thereof comprises a light chain variable region selected from the group consisting of SEQ ID NOs: 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 401, 405, 409, 413, and 417, and a homologous sequence thereof having at least 80% sequence identity yet retaining specific binding affinity to CD40.

In certain embodiments, the antibody or an antigen-binding fragment thereof comprises:
a) heavy chain variable region comprising SEQ ID NO: 247 and a light chain variable
region comprising SEQ ID NO: 249;
b) a heavy chain variable region comprising SEQ ID NO: 251 and a light chain variable region comprising SEQ ID NO: 253;
c) a heavy chain variable region comprising SEQ ID NO: 255 and a light chain variable region comprising SEQ ID NO: 257;
d) a heavy chain variable region comprising SEQ ID NO: 259 and a light chain variable region comprising SEQ ID NO: 261;
e) a heavy chain variable region comprising SEQ ID NO: 263 and a light chain variable region comprising SEQ ID NO: 265;
f) a heavy chain variable region comprising SEQ ID NO: 267 and a light chain variable region comprising SEQ ID NO: 269;
g) a heavy chain variable region comprising SEQ ID NO: 271 and a light chain variable region comprising SEQ ID NO: 273;
h) a heavy chain variable region comprising SEQ ID NO: 275 and a light chain variable region comprising SEQ ID NO: 277;
i) a heavy chain variable region comprising SEQ ID NO: 279 and a light chain variable region comprising SEQ ID NO: 281;
j) a heavy chain variable region comprising SEQ ID NO: 283 and a light chain variable region comprising SEQ ID NO: 285;
k) a heavy chain variable region comprising SEQ ID NO: 287 and a light chain variable region comprising SEQ ID NO: 289;
l) a heavy chain variable region comprising SEQ ID NO: 291 and a light chain variable region comprising SEQ ID NO: 293;
m) a heavy chain variable region comprising SEQ ID NO: 295 and a light chain variable region comprising SEQ ID NO: 297;
n) a heavy chain variable region comprising SEQ ID NO: 299 and a light chain variable region comprising SEQ ID NO: 301;
o) a heavy chain variable region comprising SEQ ID NO: 303 and a light chain variable region comprising SEQ ID NO: 305;
p) a heavy chain variable region comprising SEQ ID NO: 307 and a light chain variable region comprising SEQ ID NO: 309;
q) a heavy chain variable region comprising SEQ ID NO: 311 and a light chain variable region comprising SEQ ID NO: 313;
r) a heavy chain variable region comprising SEQ ID NO: 315 and a light chain variable region comprising SEQ ID NO: 317;
s) a heavy chain variable region comprising SEQ ID NO: 319 and a light chain variable region comprising SEQ ID NO: 321;
t) a heavy chain variable region comprising SEQ ID NO: 323 and a light chain variable region comprising SEQ ID NO: 325;
u) a heavy chain variable region comprising SEQ ID NO: 327 and a light chain variable region comprising SEQ ID NO: 329;
v) a heavy chain variable region comprising SEQ ID NO: 331 and a light chain variable region comprising SEQ ID NO: 333;
w) a heavy chain variable region comprising SEQ ID NO: 335 and a light chain variable region comprising SEQ ID NO: 337;
x) a heavy chain variable region comprising SEQ ID NO: 339 and a light chain variable region comprising SEQ ID NO: 341;
y) a heavy chain variable region comprising SEQ ID NO: 343 and a light chain variable region comprising SEQ ID NO: 345;
z) a heavy chain variable region comprising SEQ ID NO: 347 and a light chain variable region comprising SEQ ID NO: 349;
aa) a heavy chain variable region comprising SEQ ID NO: 351 and a light chain variable region comprising SEQ ID NO: 353;
bb) a heavy chain variable region comprising SEQ ID NO: 355 and a light chain variable region comprising SEQ ID NO: 357;
cc) a heavy chain variable region comprising SEQ ID NO: 359 and a light chain variable region comprising SEQ ID NO: 361;
dd) a heavy chain variable region comprising SEQ ID NO: 363 and a light chain variable region comprising SEQ ID NO: 365;
ee) a heavy chain variable region comprising SEQ ID NO: 367 and a light chain variable region comprising SEQ ID NO: 369;
ff) a heavy chain variable region comprising SEQ ID NO: 371 and a light chain variable region comprising SEQ ID NO: 373;
gg) a heavy chain variable region comprising SEQ ID NO: 375 and a light chain variable region comprising SEQ ID NO: 377;
hh) a heavy chain variable region comprising SEQ ID NO: 379 and a light chain variable region comprising SEQ ID NO: 381;
ii) a heavy chain variable region comprising SEQ ID NO: 383 and a light chain variable region comprising SEQ ID NO: 385;
jj) a heavy chain variable region comprising SEQ ID NO: 387 and a light chain variable region comprising SEQ ID NO: 389;
kk) a heavy chain variable region comprising SEQ ID NO: 391 and a light chain variable region comprising SEQ ID NO: 393;
ll) a heavy chain variable region comprising SEQ ID NO: 395 and a light chain variable region comprising SEQ ID NO: 397;
mm) a heavy chain variable region comprising SEQ ID NO: 399 and a light chain variable region comprising SEQ ID NO: 401;
nn) a heavy chain variable region comprising SEQ ID NO: 403 and a light chain variable region comprising SEQ ID NO: 405;
oo) a heavy chain variable region comprising SEQ ID NO: 407 and a light chain variable region comprising SEQ ID NO: 409;
pp) a heavy chain variable region comprising SEQ ID NO: 411 and a light chain variable region comprising SEQ ID NO: 413; or
qq) a heavy chain variable region comprising SEQ ID NO: 415 and a light chain variable region comprising SEQ ID NO: 417.

In certain embodiments, the antibody or an antigen-binding fragment thereof further comprises one or more amino acid residue substitutions or modifications yet retains specific binding affinity to CD40. In certain embodiments, at least one of the substitutions or modifications is in one or more of the CDR sequences, and/or in one or more of the heavy chain or light chain variable region sequences but not in any of the CDR sequences.

In certain embodiments, the antibody or an antigen-binding fragment thereof further comprises an immunoglobulin constant region, optionally a constant region of Ig, or optionally a constant region of human IgG.

In certain embodiments, the antibody or an antigen-binding fragment thereof is humanized.

In certain embodiments, the antibody or an antigen-binding fragment thereof is a camelized single domain antibody, a diabody, a scFv, an scFv dimer, a BsFv, a dsFv, a (dsFv)₂, a dsFv-dsFv′, an Fv fragment, a Fab, a Fab′, a F(ab′)₂, a bispecific antibody, a ds diabody, a nanobody, a domain antibody, or a bivalent antibody.

In certain embodiments, the antibody or an antigen-binding fragment thereof is capable of specifically binding to CD40, optionally CD40 derived from human or rabbit.

In certain embodiments, the antibody or an antigen-binding fragment thereof is capable of specifically binding to CD40 at a K_{D} value of no more than 7 pM, no more than 10 pM, no more than 50 pM, no more than 100 pM, no more than 200 pM, no more than 300 pM, or no more than 400 pM as measured by bio-layer interferometry.

In certain embodiments, the antibody or an antigen-binding fragment thereof is capable of specifically binding to CD40 expressed on a cell surface at an EC₅₀ of no more than 0.06 nM, no more than 0.07 nM, no more than 0.08 nM, no more than 0.09 nM, or no more than 0.1 nM as measured by flow cytometer assay.

In certain embodiments, the antibody or an antigen-binding fragment thereof is linked to one or more conjugate moieties. In certain embodiments, the conjugate moiety comprises a clearance-modifying agent, a toxin, a detectable label, a chemotherapeutic agent, or purification moiety.

In another aspect, the present disclosure provides an antibody or an antigen-binding fragment thereof, which competes for the same epitope with the antibody or antigen-binding fragment thereof provided herein.

In another aspect, the present disclosure provides a pharmaceutical composition comprising the antibody or antigen-binding fragment thereof provided herein, and a pharmaceutically acceptable carrier.

In another aspect, the present disclosure provides an isolated polynucleotide encoding the antibody or an antigen-binding fragment thereof provided herein. In certain embodiments, the isolated polynucleotide comprises a nucleotide sequence selecting from a group consisting of SEQ ID NOs: 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, and 418.

In another aspect, the present disclosure provides a vector comprising the isolated polynucleotide provided herein.

In another aspect, the present disclosure provides a host cell comprising the vector provided herein.

In another aspect, the present disclosure provides a method of expressing the antibody or antigen-binding fragment thereof provided herein, comprising culturing the host cell provided herein under the condition at which the vector provided herein is expressed.

In another aspect, the present disclosure provides a method of treating a disease or condition in a subject that would benefit from modulation of CD40 activity, comprising administering to the subject a therapeutically effective amount of the antibody or antigen-binding fragment thereof provided herein or the pharmaceutical composition provided herein. In certain embodiments, the disease or condition is a CD40 related disease or condition. In certain embodiments, the disease or condition is cancer, autoimmune disease, inflammatory disease, or infectious disease. In certain embodiments, the cancer is adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalviolar cell lung cancer, mesothelioma, head and neck cancer, squamous cell carcinoma, lymphoma, lymphocytic leukemia, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer, and Hodgkin's Disease. In certain embodiments, the subject is human. In certain embodiments, the administration is via oral, nasal, intravenous, subcutaneous, sublingual, or intramuscular administration.

In another aspect, the present disclosure provides a method of modulating CD40 activity in a CD40-expressing cell, comprising exposing the CD40-expressing cell to the antibody or antigen-binding fragment thereof provided herein.

In another aspect, the present disclosure provides a method of detecting presence or amount of CD40 in a sample, comprising contacting the sample with the antibody or antigen-binding fragment thereof provided herein, and determining the presence or the amount of CD40 in the sample.

In another aspect, the present disclosure provides a method of diagnosing a CD40 related disease or condition in a subject, comprising: a) contacting a sample obtained from the subject with the antibody or antigen-binding fragment thereof provided herein; b) determining presence or amount of CD40 in the sample; and c) correlating the presence or the amount of CD40 to existence or status of the CD40 related disease or condition in the subject.

In another aspect, the present disclosure provides use of the antibody or antigen-binding fragment thereof provided herein in the manufacture of a medicament for treating a CD40 related disease or condition in a subject.

In another aspect, the present disclosure provides use of the antibody or antigen-binding fragment thereof provided herein in the manufacture of a diagnostic reagent for diagnosing a CD40 related disease or condition.

In another aspect, the present disclosure provides a kit comprising the antibody or antigen-binding fragment thereof provided herein, useful in detecting CD40.

### BRIEF DESCRIPTION OF FIGURES

The following drawings form part of the present specification and are included to further demonstrate certain aspects of the present invention. The invention may be better understood by reference to one or more of these drawings in combination with the detailed description of specific embodiments presented herein.

FIG. 1 shows CD40 activation by the indicated anti-CD40 antibodies.

FIG. 2 shows CD40 activation by the indicated anti-CD40 antibodies after freeze-thaw treatment.

FIG. 3 shows binding of the indicated anti-CD40 antibodies to CD40 on cell surface determined by flow cytometry.

FIG. 4 shows competition of the indicated anti-CD40 antibodies with CD40L for CD40 binding, presented as percentage of CD40L binding to CD40 in the presence of the antibodies to that absent of the antibodies.

FIGS. 5A and 5B illustrate B cell activation by anti-CD40 antibodies as assessed using CD80 (FIG. 5A) and CD86 (FIG. 5B) expression. Briefly, healthy donor PBMC depleted of monocyte were incubated with IL-2 and IL-4 in the presence or absence of Anti-CD40 antibodies for 48 hrs. CD80 and CD86 expression on CD19+ cells were analyzed using flow cytometry.

FIGS. 6A-6B illustrate the dendritic cell maturation and activation by anti-CD40 antibodies as assessed using CD80 (FIG. 6A) and CD86 (FIG. 6B). Briefly, monocytes were isolated from healthy donor PBMC, differentiation toward dendritic cells were induced with GM-CSF and IL-4 for 5 days. Anti-CD40 antibodies were then introduced for additional 48 hrs. CD80 and CD86 expression monocyte derived dendritic cells (MoDC) were analyzed using flow cytometry.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. Definitions

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

The term “antigen” refers to a substance capable of inducing adaptive immune responses. Specifically, an antigen is a substance specifically bound by antibodies or T lymphocyte antigen receptors. Antigens are usually proteins and polysaccharides, less frequently also lipids. Suitable antigens include without limitation parts of bacteria (coats, capsules, cell walls, flagella, fimbrai, and toxins), viruses, and other microorganisms. Antigens also include tumor antigens, e.g., antigens generated by mutations in tumors. As used herein, antigens also include immunogens and haptens.

The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

A “Fab fragment” comprises one light chain and the C_{H}1 and variable domains of one heavy chain. The heavy chain of a Fab molecule cannot form a disulfide bond with another heavy chain molecule.

A “Fab′ fragment” comprises one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}I and C_{H}2 domains, such that an interchain disulfide bond is formed between the two heavy chains. A F(ab′)₂ fragment thus is composed of two Fab′ fragments that are held together by a disulfide bond between the two heavy chains.

“Fv” with regard to an antibody refers to the smallest fragment of the antibody to bear the complete antigen-binding site. An Fv fragment consists of the variable domain of a single light chain bound to the variable domain of a single heavy chain.

A “dsFv” refers to a disulfide-stabilized Fv fragment that the linkage between the variable domain of a single light chain and the variable domain of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

“Single-chain Fv antibody” or “scFv” refers to an engineered antibody consisting of a light chain variable domain and a heavy chain variable domain connected to one another directly or via a peptide linker sequence (Huston J S et al. Proc Natl Acad Sci USA, 85:5879(1988)).

An “Fc” region comprises two heavy chain fragments comprising the C_{H}2 and C_{H}3 domains of an antibody. The two heavy chain fragments are held together by two or more disulfide bonds and by hydrophobic interactions of the C_{H}3 domains. The Fc region of the antibody is responsible for various effector functions such as antibody-dependent cell-mediated cytotoxicity (ADCC), and complement dependent cytotoxicity (CDC), but does not function in antigen binding.

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

“Diabodies” or “dAbs” include small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes). In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “domain antibody” refers to an antibody fragment containing only the variable domain of a heavy chain or the variable domain of a light chain. In certain instances, two or more V_{H} domains are covalently joined with a peptide linker to create a bivalent or multivalent domain antibody. The two V_{H} domains of a bivalent domain antibody may target the same or different antigens.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse or rabbit. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

“CD40” as used herein, refers to CD40 derived from any vertebrate source, including mammals such as primates (e.g. humans, monkeys) and rodents (e.g., mice and rats). Exemplary sequence of human CD40 includes human CD40 protein (NCBI Ref Seq No. ALQ33424.1). Exemplary sequence of CD40 includes mouse CD40 protein (NCBI Ref Seq No. AAB08705.1); Rattus norvegicus (Rat) CD40 protein (NCBI Ref Seq No. AAH97949.1). The term “CD40” as used herein is intended to encompass any form of CD40, for example, 1) native unprocessed CD40 molecule, “full-length” CD40 chain or naturally occurring variants of CD40, including, for example, splice variants or allelic variants; 2) any form of CD40 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of CD40 subunit generated through recombinant method.

The term “anti-CD40 antibody” refers to an antibody that is capable of specifically binding to CD40 (e.g. human or mouse or rabbit CD40).

The term “specific binding” or “specifically binds” as used herein refers to a non-random binding reaction between two molecules, such as for example between an antibody and an antigen. In certain embodiments, the antibodies or antigen-binding fragments provided herein specifically bind to human and/or CD40 with a binding affinity (K_{D}) of ≤10⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹M, ≤3×10⁻⁹M, ≤2×10⁻⁹ M, or ≤10⁻⁹ M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The ability to “block binding” or “compete for the same epitope” as used herein refers to the ability of an antibody or antigen-binding fragment to inhibit the binding interaction between two molecules (e.g. human CD40 and an anti-CD40 antibody) to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

The term “epitope” as used herein refers to the specific group of atoms or amino acids on an antigen to which an antibody binds. Two antibodies may bind the same or a closely related epitope within an antigen if they exhibit competitive binding for the antigen. For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

Those skilled in the art will recognize that it is possible to determine, without undue experimentation, if a given antibody binds to the same epitope as the antibody of present disclosure by ascertaining whether the former prevents the latter from binding to a CD40 antigen polypeptide. If the given antibody competes with the antibody of present disclosure, as shown by a decrease in binding by the antibody of present disclosure to the CD40 antigen polypeptide, then the two antibodies bind to the same, or a closely related, epitope. Or if the binding of a given antibody to the CD40 antigen polypeptide was inhibited by the antibody of present disclosure, then the two antibodies bind to the same, or a closely related, epitope.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

### II. Anti-CD40 Antibody

The present disclosure provides anti-CD40 antibodies and antigen-binding fragments thereof. The anti-CD40 antibodies and antigen-binding fragments provided herein are capable of specific binding to CD40.

Binding affinity of the antibody and antigen-binding fragment provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g. K_{D}) can be appropriately determined using suitable methods known in the art, including, for example, bio-layer interferometry.

In some embodiments, the anti-CD40 antibodies and antigen-binding fragments thereof provided herein are capable of specifically binding to human CD40 with a binding affinity (K_{D}) of no more than 7 pM, no more than 10 pM, no more than 50 pM, no more than 100 pM, no more than 200 pM, no more than 300 pM, or no more than 400 pM as measured by bio-layer interferometry.

Binding of the antibodies to human CD40 can also be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assay. In certain embodiments, the antibodies and the fragments thereof provided herein specifically bind to CD40 expressed on a cell surface at an EC₅₀ (i.e. 50% binding concentration) of no more than 0.06 nM, no more than 0.07 nM, no more than 0.08 nM, no more than 0.09 nM, or no more than 0.1 nM as measured by flow cytometry assay.

In certain embodiments, the antibodies and the fragments thereof provided herein have a specific binding affinity to human CD40 which is sufficient to provide for diagnostic and/or therapeutic use.

In certain embodiments, the antibodies and the fragments thereof provided herein competes with CD40 ligands for binding to CD40.

Naturally occurring ligand of CD40 is CD40L (also referred to as CD154, gp39, and TRAP), a TNF superfamily member. CD40L is a transmembrane protein expressed predominantly on activated CD4⁺ T cells and a small subset of CD8⁺ T cells (Reviewed by (Van Kooten C. and Banchereau, 2000). CD40L exists on such cells as a trimeric structure, which induces oligomerization of its receptor upon binding.

The interaction of CD40 with CD40L induces both humoral and cell-mediated immune responses. CD40 regulates this ligand-receptor pair to activate B cells and other antigen-presenting cells (APC) including dendritic cells (DCs) (see, Toubi and Shoenfeld, 2004; Kiener, et al., 1995). Activation of CD40 on B cells induces proliferation, immunoglobulin class switching, antibody secretion, and also has a role in the development of germinal centers and the survival of memory B cells, all of which are essential to humoral immune responses (Kehry M R. J Immunol 1996; 156: 2345-2348). Binding of CD40L to CD40 on dendritic cells induces DC maturation as manifested by increasing expression of co-stimulatory molecules such as B7 family (CD80, CD86) and production of proinflammatory cytokines such as interleukin 12. These lead to potent T cell responses (Stout, R. D., J. Suttles. 1996. Immunol. Today 17:487-492; Brendan O'Sullivan, Ranjeny Thomas. Critical Reviews in Immunology 2003; 23: 83-107; Cella, M., D. Scheidegger, K. Palmer-Lehmann, P. Lane, A. Lanzavecchia, G. Alber. J. Exp. Med. 1996; 184:747-452).

CD40-CD40L plays a crucial role in driving an efficient T cell-dependent immune response. By competing with CD40L for binding to CD40, the antibodies and the fragments thereof provided herein block binding and interaction of CD40-CD40L and block CD40 signaling, and thereby provide the activity of suppressing a pathogenic autoimmune response.

In certain embodiments, binding of the antibody and the fragment thereof provided herein to CD40 on dendritic cells induces DC maturation as manifested by increasing expression of co-stimulatory molecules such as CD80, CD83, CD86. These lead to potent T cell responses (see Stout, R. D., J. Suttles. 1996. Immunol. Today 17:487-492; Brendan O'Sullivan, Ranjeny Thomas. Critical Reviews in Immunology 2003; 23: 83-107; Cella, M., D. Scheidegger, K. Palmer-Lehmann, P. Lane, A. Lanzavecchia, G. Alber. J. Exp. Med. 1996; 184:747-452). In certain embodiments, binding of the antibody and the fragment thereof provided herein to CD40 induces DC activation measured as upregulation of co-stimulatory molecules such as CD80, CD83, CD86. In certain embodiments, binding of the antibody and the fragment thereof provided herein to CD40 induces B cell activation measured as upregulation of co-stimulatory molecules such as B7 family (CD80, CD86).

### III. Specific Anti-CD40 Antibodies

The present disclosure provides anti-CD40 antibodies and antigen-binding fragments thereof comprising one or more (e.g. 1, 2, 3, 4, 5, or 6) CDR sequences of an anti-CD40 antibody clone 1, 3, 5, 6, 12, 16, 17, 24, 26, 27, 31, 45, 58, 70, 78, 86, 91, 93, 94, 102, 103, 105, 108, 109, 110, 114, 120, 121, 129, 134, 151, 166, 167, 193, 217, 233, 169a, 176a, 181a, 183, 184, 5-z, or 6-z.

Antibody clone 1 as used herein refers to a rabbit monoclonal antibody having a heavy chain (1H2) variable region of SEQ ID NO: 247, and a light chain (1L1) variable region of SEQ ID NO: 249.

Antibody clone 3 as used herein refers to a rabbit monoclonal antibody having a heavy chain (3H1) variable region of SEQ ID NO: 251, and a light chain (3L2) variable region of SEQ ID NO: 253.

Antibody clone 5 as used herein refers to a rabbit monoclonal antibody having a heavy chain (5H2) variable region of SEQ ID NO: 255, and a light chain (5L2) variable region of SEQ ID NO: 257.

Antibody clone 6 as used herein refers to a rabbit monoclonal antibody having a heavy chain (6H2) variable region of SEQ ID NO: 259, and a light chain (6L2) variable region of SEQ ID NO: 261.

Antibody clone 12 as used herein refers to a rabbit monoclonal antibody having a heavy chain (12H1) variable region of SEQ ID NO: 263, and a light chain (12L1) variable region of SEQ ID NO: 265.

Antibody clone 16 as used herein refers to a rabbit monoclonal antibody having a heavy chain (16H2) variable region of SEQ ID NO: 267, and a light chain (16L1) variable region of SEQ ID NO: 269.

Antibody clone 17 as used herein refers to a rabbit monoclonal antibody having a heavy chain (17H1) variable region of SEQ ID NO: 271, and a light chain (17L1) variable region of SEQ ID NO: 273.

Antibody clone 24 as used herein refers to a rabbit monoclonal antibody having a heavy chain (24H1) variable region of SEQ ID NO: 275, and a light chain (24L1) variable region of SEQ ID NO: 277.

Antibody clone 26 as used herein refers to a rabbit monoclonal antibody having a heavy chain (26H1) variable region of SEQ ID NO: 279, and a light chain (26L1) variable region of SEQ ID NO: 281.

Antibody clone 27 as used herein refers to a rabbit monoclonal antibody having a heavy chain (27H1) variable region of SEQ ID NO: 283, and a light chain (27L2) variable region of SEQ ID NO: 285.

Antibody clone 31 as used herein refers to a rabbit monoclonal antibody having a heavy chain (31H2) variable region of SEQ ID NO: 287, and a light chain (31L1) variable region of SEQ ID NO: 289.

Antibody clone 45 as used herein refers to a rabbit monoclonal antibody having a heavy chain (45H1) variable region of SEQ ID NO: 291, and a light chain (45L2) variable region of SEQ ID NO: 293.

Antibody clone 58 as used herein refers to a rabbit monoclonal antibody having a heavy chain (58H2) variable region of SEQ ID NO: 295, and a light chain (58L1) variable region of SEQ ID NO: 297.

Antibody clone 70 as used herein refers to a rabbit monoclonal antibody having a heavy chain (70H1) variable region of SEQ ID NO: 299, and a light chain (70L2) variable region of SEQ ID NO: 301.

Antibody clone 78 as used herein refers to a rabbit monoclonal antibody having a heavy chain (78H2) variable region of SEQ ID NO: 303, and a light chain (78L1) variable region of SEQ ID NO: 305.

Antibody clone 86 as used herein refers to a rabbit monoclonal antibody having a heavy chain (86H2) variable region of SEQ ID NO: 307, and a light chain (86L2) variable region of SEQ ID NO: 309.

Antibody clone 91 as used herein refers to a rabbit monoclonal antibody having a heavy chain (91H1) variable region of SEQ ID NO: 311, and a light chain (91L1) variable region of SEQ ID NO: 313.

Antibody clone 93 as used herein refers to a rabbit monoclonal antibody having a heavy chain (93H2) variable region of SEQ ID NO: 315, and a light chain (93L2) variable region of SEQ ID NO: 317.

Antibody clone 94 as used herein refers to a rabbit monoclonal antibody having a heavy chain (94H1) variable region of SEQ ID NO: 319, and a light chain (94L2) variable region of SEQ ID NO: 321.

Antibody clone 102 as used herein refers to a rabbit monoclonal antibody having a heavy chain (102H1) variable region of SEQ ID NO: 323, and a light chain (102L1) variable region of SEQ ID NO: 325.

Antibody clone 103 as used herein refers to a rabbit monoclonal antibody having a heavy chain (103H2) variable region of SEQ ID NO: 327, and a light chain (103L2) variable region of SEQ ID NO: 329.

Antibody clone 105 as used herein refers to a rabbit monoclonal antibody having a heavy chain (105H1) variable region of SEQ ID NO: 331, and a light chain (105L4) variable region of SEQ ID NO: 333.

Antibody clone 108 as used herein refers to a rabbit monoclonal antibody having a heavy chain (108H1) variable region of SEQ ID NO: 335, and a light chain (108L3) variable region of SEQ ID NO: 337.

Antibody clone 109 as used herein refers to a rabbit monoclonal antibody having a heavy chain (109H2) variable region of SEQ ID NO: 339, and a light chain (109L1) variable region of SEQ ID NO: 341.

Antibody clone 110 as used herein refers to a rabbit monoclonal antibody having a heavy chain (110H1) variable region of SEQ ID NO: 343, and a light chain (110L1) variable region of SEQ ID NO: 345.

Antibody clone 114 as used herein refers to a rabbit monoclonal antibody having a heavy chain (114H2) variable region of SEQ ID NO: 347, and a light chain (114L1) variable region of SEQ ID NO: 349.

Antibody clone 120 as used herein refers to a rabbit monoclonal antibody having a heavy chain (120H1) variable region of SEQ ID NO: 351, and a light chain (120L1) variable region of SEQ ID NO: 353.

Antibody clone 121 as used herein refers to a rabbit monoclonal antibody having a heavy chain (121H2) variable region of SEQ ID NO: 355, and a light chain (121L1) variable region of SEQ ID NO: 357.

Antibody clone 129 as used herein refers to a rabbit monoclonal antibody having a heavy chain (129H1) variable region of SEQ ID NO: 359, and a light chain (129L1) variable region of SEQ ID NO: 361.

Antibody clone 134 as used herein refers to a rabbit monoclonal antibody having a heavy chain (134H1) variable region of SEQ ID NO: 363, and a light chain (134L2) variable region of SEQ ID NO: 365.

Antibody clone 151 as used herein refers to a rabbit monoclonal antibody having a heavy chain (151H1) variable region of SEQ ID NO: 367, and a light chain (151L1) variable region of SEQ ID NO: 369.

Antibody clone 166 as used herein refers to a rabbit monoclonal antibody having a heavy chain (166H2) variable region of SEQ ID NO: 371, and a light chain (166L1) variable region of SEQ ID NO: 373.

Antibody clone 167 as used herein refers to a rabbit monoclonal antibody having a heavy chain (167H2) variable region of SEQ ID NO: 375, and a light chain (167L2) variable region of SEQ ID NO: 377.

Antibody clone 193 as used herein refers to a rabbit monoclonal antibody having a heavy chain (193H1) variable region of SEQ ID NO: 379, and a light chain (193L2) variable region of SEQ ID NO: 381.

Antibody clone 217 as used herein refers to a rabbit monoclonal antibody having a heavy chain (217H2) variable region of SEQ ID NO: 383, and a light chain (217L1) variable region of SEQ ID NO: 385.

Antibody clone 233 as used herein refers to a rabbit monoclonal antibody having a heavy chain (233H1) variable region of SEQ ID NO: 387, and a light chain (233L1) variable region of SEQ ID NO: 389.

Antibody clone 169a as used herein refers to a rabbit monoclonal antibody having a heavy chain (169aH1) variable region of SEQ ID NO: 391, and a light chain (169aL1) variable region of SEQ ID NO: 393.

Antibody clone 176a as used herein refers to a rabbit monoclonal antibody having a heavy chain (176aH1) variable region of SEQ ID NO: 395, and a light chain (176aL1) variable region of SEQ ID NO: 397.

Antibody clone 181a as used herein refers to a rabbit monoclonal antibody having a heavy chain (181aH1) variable region of SEQ ID NO: 399, and a light chain (181aL1) variable region of SEQ ID NO: 401.

Antibody clone 183a as used herein refers to a rabbit monoclonal antibody having a heavy chain (183aH2) variable region of SEQ ID NO: 403, and a light chain (183aL1) variable region of SEQ ID NO: 405.

Antibody clone 184a as used herein refers to a rabbit monoclonal antibody having a heavy chain (184aH1) variable region of SEQ ID NO: 407, and a light chain (184aL1) variable region of SEQ ID NO: 409.

Antibody clone 5-z as used herein refers to a humanized antibody based on antibody 5 that comprises a heavy chain (5H2-z) variable region of SEQ ID NO: 411, and a light chain (5L2-z) variable region of SEQ ID NO: 413. Antibody 5-z has comparable affinity to the antigen as compared with its parent antibody 5.

Antibody clone 6-z as used herein refers to a humanized antibody based on antibody 6 that comprises a heavy chain (6H2-z) variable region of SEQ ID NO: 415, and a light chain (6L2-z) variable region of SEQ ID NO: 417. Antibody 6-z has comparable affinity to the antigen as compared with its parent antibody 6.

Table 1 shows the CDR sequences of these 43 anti-CD40 antibodies.

TABLE 1

| Antibody clone # | | CDR1 | CDR2 | CDR3 |
|---|---|---|---|---|
| 1 | 1H2 | SEQ ID NO: 1 | SEQ ID NO: 3 | SEQ ID NO: 5 |
| | | SSNAI | GTIYADDNTYY | KGASYYPL |
| | | | ANWAR | |
| | 1L1 | SEQ ID NO: 2 | SEQ ID NO: 4 | SEQ ID NO: 6 |
| | | QASQSIGSYLA | RASTLAS | LGWHTYTDDGTH |
| 3 | 3H1 | SEQ ID NO: 7 | SEQ ID NO: 9 | SEQ ID NO: 11 |
| | | SSNAI | GYMDPERNIY | RGVTYYSM |
| | | | YANWSK | |
| | 3L2 | SEQ ID NO: 8 | SEQ ID NO: 10 | SEQ ID NO: 12 |
| | | QASQSIDNRYLS | KASTLAS | QGGYYGNSYVGA |
| 5 | 5H2 | SEQ ID NO: 13 | SEQ ID NO: 15 | SEQ ID NO: 17 |
| | | SSNAI | GIIYASGDTYY | RGYTTLYF |
| | | | ASWAX | |
| | 5L2 | SEQ ID NO: 14 | SEQ ID NO: 16 | SEQ ID NO: 18 |
| | | QASESISTRLA | SASTLPS | QGGYSSGAGTA |
| 6 | 6H2 | SEQ ID NO: 19 | SEQ ID NO: 21 | SEQ ID NO: 23 |
| | | SRYHM | GIIYVSDNTYY | RVGSFWSSKL |
| | | | ATWAK | |
| | 6L2 | SEQ ID NO: 20 | SEQ ID NO: 22 | SEQ ID NO: 24 |
| | | QASENIYSSLA | EASNLES | QSTYFGNSYVFA |
| 12 | 12H1 | SEQ ID NO: 25 | SEQ ID NO: 27 | SEQ ID NO: 29 |
| | | SSNAV | GVISPGDDIYY | RGFSYSAL |
| | | | ANWAK | |
| | 12L1 | SEQ ID NO: 26 | SEQ ID NO: 28 | SEQ ID NO: 30 |
| | | QSSQSVYSNWLS | QASKVPS | QGTYDGSGWSNA |
| 16 | 16H2 | SEQ ID NO: 31 | SEQ ID NO: 33 | SEQ ID NO: 35 |
| | | SSNAM | GIISNSGSTYYA | RGFRYPNP |
| | | | SWAK | |
| | 16L1 | SEQ ID NO: 32 | SEQ ID NO: 34 | SEQ ID NO: 36 |
| | | QASESVGNNNY | DASRLAS | LGGYVSSGWYGA |
| | | LS | | |
| 17 | 17H1 | SEQ ID NO: 37 | SEQ ID NO: 39 | SEQ ID NO: 41 |
| | | SSNAL | ASIYAGGDTYY | RGAMTYSL |
| | | | ATWAX | |
| | 17L1 | SEQ ID NO: 38 | SEQ ID NO: 40 | SEQ ID NO: 42 |
| | | QASQSVHNNNY | QASKLAS | QSYYYSGSSGAV |
| | | LS | | NS |
| 24 | 24H1 | SEQ ID NO: 43 | SEQ ID NO: 45 | SEQ ID NO: 47 |
| | | SSNAI | GVIYAGGGAF | RGYTYLAF |
| | | | YANWAK | |
| | 24L1 | SEQ ID NO: 44 | SEQ ID NO: 46 | SEQ ID NO: 48 |
| | | QASQSISNLIS | KASTLAS | QGSAYGTSDVCA |
| 26 | 26H1 | SEQ ID NO: 49 | SEQ ID NO: 51 | SEQ ID NO: 53 |
| | | SSNAM | GAIDANGSPYY | RGYTRLDL |
| | | | TNWAK | |
| | 26L1 | SEQ ID NO: 50 | SEQ ID NO: 52 | SEQ ID NO: 54 |
| | | QSSQSILSDNYL | QASKLVS | QGAYDSSDWYGA |
| | | A | | |
| 27 | 27H1 | SEQ ID NO: 55 | SEQ ID NO: 57 | SEQ ID NO: 59 |
| | | NNYAM | GFINSGGSAYY | RGVPKMDL |
| | | | ASWAK | |
| | 27L2 | SEQ ID NO: 56 | SEQ ID NO: 58 | SEQ ID NO: 60 |
| | | QASQSVYNGNE | AASILAS | AGYQSSVIDDIG |
| | | LS | | |
| 31 | 31H2 | SEQ ID NO: 61 | SEQ ID NO: 63 | SEQ ID NO: 65 |
| | | NNYAM | GFINTGDRAYY | RGVPAMGL |
| | | | ASWAK | |
| | 31L1 | SEQ ID NO: 62 | SEQ ID NO: 64 | SEQ ID NO: 66 |
| | | QASQSVYNNNE | AASYVAS | AGYESSGIDDIG |
| | | LS | | |
| 45 | 45H1 | SEQ ID NO: 67 | SEQ ID NO: 69 | SEQ ID NO: 71 |
| | | SSNAM | GIIYASGSTYY | RGFARLPL |
| | | | ASWAK | |
| | 45L2 | SEQ ID NO: 68 | SEQ ID NO: 70 | SEQ ID NO: 72 |
| | | QSSQSVGSNYLS | DASTLAS | QGSYYSSDWYGA |
| 58 | 58H2 | SEQ ID NO: 73 | SEQ ID NO: 75 | SEQ ID NO: 77 |
| | | NTNYYM | ACSYTTSGSTY | KYGAGYTYNL |
| | | | YATWAK | |
| | 58L1 | SEQ ID NO: 74 | SEQ ID NO: 76 | SEQ ID NO: 78 |
| | | QASESISNYLA | RASTLES | QQGYSNTNLDNI |
| 70 | 70H1 | SEQ ID NO: 79 | SEQ ID NO: 81 | SEQ ID NO: 83 |
| | | SSNAI | GWIDATGSAY | RGFRYSAF |
| | | | YATWAK | |
| | 70L2 | SEQ ID NO: 80 | SEQ ID NO: 82 | SEQ ID NO: 84 |
| | | QASQSVYNNNY | DTSTLAS | AGTYSTSDWSVA |
| | | LS | | |
| 78 | 78H2 | SEQ ID NO: 85 | SEQ ID NO: 87 | SEQ ID NO: 89 |
| | | STYFM | GWINTNDKIYY | SPYPRYASGLNL |
| | | | ASWAK | |
| | 78L1 | SEQ ID NO: 86 | SEQ ID NO: 88 | SEQ ID NO: 90 |
| | | QASQSIHNYLA | SASNLAS | QCTYYGSSYENT |
| 86 | 86H2 | SEQ ID NO: 91 | SEQ ID NO: 93 | SEQ ID NO: 95 |
| | | SRYHM | ATTHIDGGVYY | RKFDL |
| | | | AIWAK | |
| | 86L2 | SEQ ID NO: 92 | SEQ ID NO: 94 | SEQ ID NO: 96 |
| | | QSSESVSNNNW | QASKLAS | QGGYYDSGWYY |
| | | LS | | A |
| 91 | 91H1 | SEQ ID NO: 97 | SEQ ID NO: 99 | SEQ ID NO: 101 |
| | | SSNAI | GFIDSEGSASY | RGFRYLPL |
| | | | ASWAN | |
| | 91L1 | SEQ ID NO: 98 | SEQ ID NO: 100 | SEQ ID NO: 102 |
| | | QASQSVYYNNY | DTSKLAS | QGTYYSSGWYW |
| | | LA | | NA |
| 93 | 93H2 | SEQ ID NO: 103 | SEQ ID NO: 105 | SEQ ID NO: 107 |
| | | SSNAI | GVIYAGGGAF | RGYTYLAF |
| | | | YASWAK | |
| | 93L2 | SEQ ID NO: 104 | SEQ ID NO: 106 | SEQ ID NO: 108 |
| | | QASQSINNFLS | KASTLAS | QGSAYGTSDVCA |
| 94 | 94H1 | SEQ ID NO: 109 | SEQ ID NO: 111 | SEQ ID NO: 113 |
| | | SSNAL | ASIYAGGDTYY | RGAMTYSL |
| | | | ATWAK | |
| | 94L2 | SEQ ID NO: 110 | SEQ ID NO: 112 | SEQ ID NO: 114 |
| | | QASQSVHNNNY | QASKLAS | QSYYYSGCSGAV |
| | | LS | | NS |
| 102 | 102H1 | SEQ ID NO: 115 | SEQ ID NO: 117 | SEQ ID NO: 119 |
| | | SSNAV | GVISVSGNIYY | RPWDL |
| | | | ANWAR | |
| | 102L1 | SEQ ID NO: 116 | SEQ ID NO: 118 | SEQ ID NO: 120 |
| | | QASESISSWLA | LASTLAS | QCSSYTSGYVAA |
| 103 | 103H2 | SEQ ID NO: 121 | SEQ ID NO: 123 | SEQ ID NO: 125 |
| | | NSFHM | GVIHPNDATYY | RDLAGYSTGGSF |
| | | | ASWAK | |
| | 103L2 | SEQ ID NO: 122 | SEQ ID NO: 124 | SEQ ID NO: 126 |
| | | QASQSVYNNNW | RASTLAS | AAYKSWSNDDFG |
| | | LS | | |
| 105 | 105H1 | SEQ ID NO: 127 | SEQ ID NO: 129 | SEQ ID NO: 131 |
| | | SSVAM | GVISTSGNKYY | RAWNL |
| | | | ATWAK | |
| | 105L4 | SEQ ID NO: 128 | SEQ ID NO: 130 | SEQ ID NO: 132 |
| | | RASEDIESYLA | RASKLAS | QCTTYTSTYVGG |
| | | | | G |
| 108 | 108H1 | SEQ ID NO: 133 | SEQ ID NO: 135 | SEQ ID NO: 137 |
| | | SSNAM | GFIDAGGSAYY | KGLSWSDF |
| | | | ATWVN | |
| | 108L3 | SEQ ID NO: 134 | SEQ ID NO: 136 | SEQ ID NO: 138 |
| | | QSSQSVVSNNRL | LASTLPS | QGTYGSSSYYGA |
| | | S | | |
| 109 | 109H2 | SEQ ID NO: 139 | SEQ ID NO: 141 | SEQ ID NO: 143 |
| | | SSFHM | GVIHPNDITYY | RDLTGGTTGGRL |
| | | | ASWAK | |
| | 109L1 | SEQ ID NO: 140 | SEQ ID NO: 142 | SEQ ID NO: 144 |
| | | QSSKSVCNNDW | RASTLAS | AGYASWNNDDFG |
| | | LS | | |
| 110 | 110H1 | SEQ ID NO: 145 | SEQ ID NO: 147 | SEQ ID NO: 149 |
| | | SSYWM | GIISTSENIYYA | RWSDL |
| | | | TWAK | |
| | 110L1 | SEQ ID NO: 146 | SEQ ID NO: 148 | SEQ ID NO: 150 |
| | | SSQSVGSGNILS | QASKLAS | LGSYGCSSADCA |
| | | | | A |
| 114 | 114H2 | SEQ ID NO: 151 | SEQ ID NO: 153 | SEQ ID NO: 155 |
| | | SSNAI | GIIDSNGSTYY | RGAIYPAL |
| | | | ASWAX | |
| | 114L1 | SEQ ID NO: 152 | SEQ ID NO: 154 | SEQ ID NO: 156 |
| | | QASQSISSSYLS | KASTLAS | LYAYFGGSTAEH |
| | | | | T |
| 120 | 120H1 | SEQ ID NO: 157 | SEQ ID NO: 159 | SEQ ID NO: 161 |
| | | SRYYM | ATTHIDGGVYY | RKFDL |
| | | | ANWAK | |
| | 120L1 | SEQ ID NO: 158 | SEQ ID NO: 160 | SEQ ID NO: 162 |
| | | QSSESVSNNNW | AASKLAS | QGGYYDSGWYY |
| | | LS | | A |
| 121 | 12H12 | SEQ ID NO: 163 | SEQ ID NO: 165 | SEQ ID NO: 167 |
| | | SSNAI | GIIDAPGSTYY | RNYAYFAL |
| | | | ASWAK | |
| | 121L1 | SEQ ID NO: 164 | SEQ ID NO: 166 | SEQ ID NO: 168 |
| | | QASESVGSNNRL | EASKLPS | LGWHASTDDGW |
| | | S | | A |
| 129 | 129H1 | SEQ ID NO: 169 | SEQ ID NO: 171 | SEQ ID NO: 173 |
| | | SSNAI | GIIWSGGDTDY | KGATYSAL |
| | | | ATWAK | |
| | 129L1 | SEQ ID NO: 170 | SEQ ID NO: 172 | SEQ ID NO: 174 |
| | | QASQSISSNYYA | KASTLAS | QGFDYGNSNVGA |
| 134 | 134H1 | SEQ ID NO: 175 | SEQ ID NO: 177 | SEQ ID NO: 179 |
| | | SSNAM | GYIWSGGNTD | RGGSYFPF |
| | | | YASWAK | |
| | 134L2 | SEQ ID NO: 176 | SEQ ID NO: 178 | SEQ ID NO: 180 |
| | | QSSQSVYIDRLA | QASKLPS | AGFYDSGSGTYTL |
| | | | | A |
| 151 | 151H1 | SEQ ID NO: 181 | SEQ ID NO: 183 | SEQ ID NO: 185 |
| | | SSNAI | GVIDAGGSTYF | RGWSRHDF |
| | | | ASWAK | |
| | 151L1 | SEQ ID NO: 182 | SEQ ID NO: 184 | SEQ ID NO: 186 |
| | | QASQSISNILA | SASTLAS | QGYDSTVGVGA |
| 166 | 166H2 | SEQ ID NO: 187 | SEQ ID NO: 189 | SEQ ID NO: 191 |
| | | SRYHM | GIIYVSDDSYY | RVGSVWSSKL |
| | | | ASWAK | |
| | 166L1 | SEQ ID NO: 188 | SEQ ID NO: 190 | SEQ ID NO: 192 |
| | | QASENIYNNLA | RASTLES | QSTYFGGSYVFA |
| 167 | 167H2 | SEQ ID NO: 193 | SEQ ID NO: 195 | SEQ ID NO: 197 |
| | | SSNAI | GTIYATDSTSY | LGASYSAL |
| | | | ASWAK | |
| | 167L2 | SEQ ID NO: 194 | SEQ ID NO: 196 | SEQ ID NO: 198 |
| | | QASESVASNDRL | QASTLAS | AGYKSSSTDGNA |
| | | A | | |
| 193 | 193H1 | SEQ ID NO: 199 | SEQ ID NO: 201 | SEQ ID NO: 203 |
| | | SSYDM | GVIATGGRRDY | RYSDSDGYAL |
| | | | ASWAK | |
| | 193L2 | SEQ ID NO: 200 | SEQ ID NO: 202 | SEQ ID NO: 204 |
| | | QASESIGSWLA | SASTLAF | QSNYYSTSGHA |
| 217 | 217H2 | SEQ ID NO: 205 | SEQ ID NO: 207 | SEQ ID NO: 209 |
| | | SNYWM | GTINYGGSTYY | RDNGAYTFDS |
| | | | ASWAK | |
| | 217L1 | SEQ ID NO: 206 | SEQ ID NO: 208 | SEQ ID NO: 210 |
| | | QASQSVYNNNY | AASTLAS | LGKSSCSYDDCR |
| | | LS | | A |
| 233 | 233H1 | SEQ ID NO: 211 | SEQ ID NO: 213 | SEQ ID NO: 215 |
| | | SYYSWA | ACIDGGGSRAT | RSDYNGYISYFDL |
| | | | YYASWAK | |
| | 233L1 | SEQ ID NO: 212 | SEQ ID NO: 214 | SEQ ID NO: 216 |
| | | QASQSIRSDLA | KASTLAS | QSYYHSSSTA |
| 169a | 169aH1 | SEQ ID NO: 217 | SEQ ID NO: 219 | SEQ ID NO: 221 |
| | | SSNAI | GYIDANTNAY | RGVTYYPM |
| | | | YASWAK | |
| | 169aL1 | SEQ ID NO: 218 | SEQ ID NO: 220 | SEQ ID NO: 222 |
| | | QASQSIASRYCS | KASTLAS | QGGYYGDSYVGA |
| 176a | 176aH1 | SEQ ID NO: 223 | SEQ ID NO: 225 | SEQ ID NO: 227 |
| | | SSNAI | GAIYSDDNTYY | RGASRFDF |
| | | | ANWAK | |
| | 176aL1 | SEQ ID NO: 224 | SEQ ID NO: 226 | SEQ ID NO: 228 |
| | | QASQSINGNYLA | KASTLTS | QYTDYGSTYVGA |
| 181a | 181aH1 | SEQ ID NO: 229 | SEQ ID NO: 231 | SEQ ID NO: 233 |
| | | SSANI | GYIDANTNAY | RGVTYYPM |
| | | | YASWAK | |
| | 181aL1 | SEQ ID NO: 230 | SEQ ID NO: 232 | SEQ ID NO: 234 |
| | | QASQSIGSRYWS | KASTLAS | QGGYYGDSYVGA |
| 183 | 183aH2 | SEQ ID NO: 235 | SEQ ID NO: 237 | SEQ ID NO: 239 |
| | | SSNAM | GIIYASDSTYY | RGATYIPL |
| | | | ASWAK | |
| | 183aL1 | SEQ ID NO: 236 | SEQ ID NO: 238 | SEQ ID NO: 240 |
| | | QASQSISSSYLA | KASTLAS | QCTDYGSSYVGT |
| 184a | 184aH1 | SEQ ID NO: 241 | SEQ ID NO: 243 | SEQ ID NO: 245 |
| | | SSNTM | GLIGPVSNTYY | RGWFQYSF |
| | | | ANWAK | |
| | 184aL1 | SEQ ID NO: 242 | SEQ ID NO: 244 | SEQ ID NO: 246 |
| | | QASQSIDSYLS | KASTLAS | QGGYYSSSNNYIT |
| 5-z | 5H2-z | SEQ ID NO: 13 | SEQ ID NO: 15 | SEQ ID NO: 17 |
| | | SSNAI | GIIYASGDTYY | RGYTTLYF |
| | | | ASWAK | |
| | 5L2-z | SEQ ID NO: 14 | SEQ ID NO: 16 | SEQ ID NO: 18 |
| | | QASESISTRLA | SASTLPS | QGGYSSGAGTA |
| 6-z | 6H2-z | SEQ ID NO: 19 | SEQ ID NO: 21 | SEQ ID NO: 23 |
| | | SRYHM | GIIYVSDNTYY | RVGSFWSSKL |
| | | | ATWAX | |
| | 6L2-z | SEQ ID NO: 20 | SEQ ID NO: 22 | SEQ ID NO: 24 |
| | | QASENIYSSLA | EASNLES | QSTYFGNSYVFA |

The heavy chain and light chain variable region sequences of the 43 anti-CD40 antibodies above are provided below.

1H2
Amino acid sequence (SEQ ID NO: 247):
QSLEESGGRLVTPGTPLTLTCTVSGFSLSSNAISWVRQAPGKGLEWIGTIYADDNTYY
ANWARGRFTISRTSTTVDLKITSPTTEDTATYFCAKGASYYPLWGPGTLVTVSS
Nucleic acid sequence (SEQ ID NO: 248):
CAGTCGCTGGAGGAGTCCGGGGGTCGCCTGGTCACGCCTGGGACACCCCTGACA
CTCACCTGCACAGTCTCTGGATTCTCCCTCAGTAGCAATGCAATAAGCTGGGTCC
GCCAGGCTCCAGGGAAGGGGCTGGAATGGATCGGAACCATTTATGCTGATGATA
ACACATATTACGCGAACTGGGCGAGAGGCCGGTTCACCATCTCCAGAACCTCGA
CCACGGTGGATCTGAAAATCACCAGTCCGACAACCGAGGACACGGCCACCTATT
TCTGTGCCAAAGGTGCTTCTTATTATCCTTTGTGGGGCCCAGGCACCCTGGTCACC
GTCTCCTCA
1L1
Amino acid sequence (SEQ ID NO: 249):
VVMTQTPSSTSAAVEGTVTINCQASQSIGSYLAWFQQKPGQPPKWYRASTLASGVP
SRFKGSGSGTQFTLTISGVQREDAATYYCLGWHTYTDDGTHFGGGTEVVVK
Nucleic acid sequence (SEQ ID NO: 250):
GTCGTGATGACCCAGACTCCATCCTCCACGTCTGCCGCTGTGGAAGGCACAGTCA
CCATCAATTGCCAGGCCAGTCAGAGCATTGGTAGCTATTTGGCCTGGTTTCAGCA
GAAACCAGGGCAGCCTCCCAAGCTCCTGATCTACAGGGCTTCCACTCTGGCATCT
GGGGTCCCATCGCGGTTCAAAGGCAGTGGATCTGGGACACAGTTCACTCTCACCA
TCAGCGGCGTGCAGCGTGAGGATGCTGCCACTTACTACTGTCTAGGCTGGCATAC
TTATACTGATGATGGAACTCATTTCGGCGGAGGGACCGAGGTGGTGGTCAAA
3H1
Amino acid sequence (SEQ ID NO: 251):
QSVEESGGRLVTPGTPLTLTCTVSGFSLSSNAISWVRQAPGKGLEWIGYMDPERNIYY
ANWSKGRFTFSQTSTTVDLKIASPTSEDTATYFCARGVTYYSMWGPGTLVTVSS
Nucleic acid sequence (SEQ ID NO: 252):
CAGTCGGTGGAGGAGTCCGGGGGTCGCCTGGTCACGCCTGGGACACCCCTGACA
CTCACCTGCACCGTCTCTGGATTCTCCCTCAGTAGCAATGCAATAAGCTGGGTCC
GCCAGGCTCCAGGAAAGGGGCTGGAGTGGATCGGATACATGGATCCTGAGAGAA
ACATATACTACGCGAATTGGTCAAAAGGCCGATTCACCTTCTCCCAAACCTCGAC
CACGGTGGATCTGAAAATCGCCAGTCCGACAAGCGAGGACACGGCCACCTATTT
CTGTGCCAGAGGTGTTACTTATTATTCAATGTGGGGCCCGGGCACCCTGGTCACC
GTCTCCTCA
+
3L2
Amino acid sequence (SEQ ID NO: 253):
DVVMTQTPASVSEPVGGTVTIKCQASQSIDNRYLSWYQQKPGQPPKWYKASTLAS
GVSSRFKGSGSGTEFTLTISDLECADAATYYCQGGYYGNSYVGAFGGGTEVVVK
Nucleic acid sequence (SEQ ID NO: 254):
GATGTTGTGATGACCCAGACTCCAGCCTCCGTGTCTGAACCTGTGGGAGGCACAG
TCACCATCAAGTGCCAGGCCAGTCAGAGTATTGATAATAGGTACTTATCCTGGTA
TCAGCAGAAACCAGGGCAGCCTCCCAAGCTCCTGATCTACAAGGCATCCACTCTG
GCATCTGGGGTCTCATCGCGGTTCAAAGGCAGTGGATCTGGGACAGAGTTCACTC
TCACCATCAGCGACCTGGAGTGTGCCGATGCTGCCACTTACTACTGTCAAGGCGG
TTATTATGGTAATAGTTATGTTGGCGCTTTCGGCGGAGGGACCGAGGTGGTGGTC
AAA
5H2
Amino acid sequence (SEQ ID NO: 255):
QSVKESGGGLFKPTDTLTLTCTVSGFSLSSNAISWVRQAPGKGLEWIGIIYASGDTYY
ASWAKGRFTISKTSSTTVDLKMTSLTTEGTATYFCARGYTTLYFWGPGTLVTVSS
Nucleic acid sequence (SEQ ID NO: 256):
CAGTCAGTGAAGGAGTCCGGGGGAGGTCTCTTCAAGCCAACGGATACCCTGACA
CTCACCTGCACCGTCTCTGGATTCTCCCTCAGTAGTAATGCAATAAGCTGGGTCC
GCCAGGCTCCAGGGAAGGGGCTGGAATGGATCGGAATCATTTATGCTAGTGGTG
ACACATACTACGCGAGCTGGGCGAAAGGCCGATTCACCATCTCCAAAACCTCGT
CGACCACGGTGGATCTGAAAATGACCAGTCTGACAACCGAGGGCACGGCCACCT
ATTTCTGTGCCAGAGGATATACTACTCTTTACTTCTGGGGCCCAGGCACCCTGGT
CACCGTCTCCTCA
5L2
Amino acid sequence (SEQ ID NO: 257):
QIVMTQTPASVSAAVGGTVTIKCQASESISTRLAWYQQKPGQPPKWYSASTLPSGV
PSRFSGSGSGTDFTLTISGVQCDDAATYYCQGGYSSGAGTAFGGGTEVVVK
Nucleic acid sequence (SEQ ID NO: 258):
CAAATTGTGATGACCCAGACTCCAGCCTCCGTGTCTGCAGCTGTGGGAGGCACAG
TCACCATCAAGTGCCAGGCCAGTGAGAGTATTAGTACTAGGTTAGCCTGGTATCA
GCAGAAACCAGGGCAGCCTCCCAAGCTCCTGATCTACTCTGCATCCACTCTGCCA
TCTGGGGTCCCATCGCGGTTCAGTGGCAGTGGATCTGGGACAGACTTCACTCTCA
CCATCAGCGGCGTGCAGTGTGACGATGCTGCCACTTACTACTGTCAAGGCGGTTA
TAGTAGTGGTGCTGGTACTGCTTTCGGCGGAGGGACCGAGGTGGTGGTCAAA
6H2
Amino acid sequence (SEQ ID NO: 259):
QSLEESGGRLVTPGTPLTLTCTASGFDFSRYHMCWVRQAPGKGLEWIGIIYVSDNTY
YATWAKGRFTISRTSTTVDLKITSPTTEDTATYFCVRVGSFWSSKLWGPGTLVTVSS
Nucleic acid sequence (SEQ ID NO: 260):
CAGTCGCTGGAGGAGTCCGGGGGTCGCCTGGTCACGCCTGGGACACCCCTGACA
CTCACCTGCACAGCCTCTGGATTCGACTTCAGTAGGTACCACATGTGCTGGGTCC
GCCAGGCTCCAGGGAAGGGGCTGGAATGGATCGGAATCATTTATGTTAGTGATA
ACACATACTACGCGACCTGGGCAAAAGGCCGATTCACCATCTCCAGAACCTCGA
CCACGGTGGATCTGAAAATCACCAGTCCGACAACCGAGGACACGGCCACCTATT
TCTGTGTCAGAGTTGGTAGTTTTTGGAGCAGTAAGTTGTGGGGCCCAGGCACCCT
GGTCACCGTCTCCTCA
6L2
Amino acid sequence (SEQ ID NO: 261):
DVVMTQTPASVSEPVGGTVTINCQASENIYSSLAWYQQKPGQPPKLLIYEASNLESG
VSSRFSGSGSGTEFTLTISDLECADAATYYCQSTYFGNSYVFAFGGGTEVVVK
Nucleic acid sequence (SEQ ID NO: 262):
GATGTTGTGATGACCCAGACTCCAGCCTCCGTGTCTGAACCTGTGGGAGGCACAG
TCACCATCAATTGCCAGGCCAGTGAGAACATTTACAGCTCTTTAGCCTGGTATCA
GCAGAAACCAGGGCAGCCTCCCAAGCTCCTGATCTATGAAGCATCCAATCTAGA
ATCTGGGGTCTCATCAAGGTTCAGCGGCAGTGGATCTGGGACAGAGTTCACTCTC
ACCATCAGCGACCTGGAGTGTGCCGATGCTGCCACTTACTACTGTCAATCTACTT
ATTTCGGTAATAGTTATGTTTTTGCTTTCGGCGGAGGGACCGAGGTGGTGGTCAA
A
12H1
Amino acid sequence (SEQ ID NO: 263):
QSVEESGGRLVTPGTPLTLTCTVSGFSLSSNAVNWVRQAPGEGLEWIGVISPGDDIYY
ANWAKGRFTISKTSTTVDLKITSPTTEDTATYFCARGFSYSALWGQGTLVTVSS
Nucleic acid sequence (SEQ ID NO: 264):
CAGTCGGTGGAGGAGTCCGGGGGTCGCCTGGTCACGCCTGGGACACCCCTGACA
CTCACCTGCACCGTCTCTGGATTCTCCCTCAGTAGTAATGCAGTGAACTGGGTCC
GCCAGGCTCCAGGGGAGGGGCTGGAATGGATCGGAGTCATTAGTCCTGGTGATG
ACATATACTACGCGAATTGGGCAAAAGGCCGATTCACCATCTCCAAAACCTCGA
CCACGGTGGATCTGAAAATCACCAGTCCGACAACCGAGGACACGGCCACCTATT
TCTGTGCCAGAGGTTTTTCCTATTCAGCCTTGTGGGGCCAAGGCACCCTGGTCAC
CGTCTCCTCA
12L1
Amino acid sequence (SEQ ID NO: 265):
QVLTQTASPVSAAVGGTVTINCQSSQSVYSNWLSWYQQKPGQRPKLLIYQASKVPS
GVSSRFSGSGSGTQFILTISGVQCDDAATYYCQGTYDGSGWSNAFGGGTEVVVK
Nucleic acid sequence (SEQ ID NO: 266):
CAAGTGCTGACCCAGACTGCATCGCCCGTGTCTGCCGCTGTGGGAGGCACAGTCA
CCATCAACTGCCAGTCCAGTCAGAGTGTTTATAGTAACTGGCTATCCTGGTATCA
GCAGAAACCAGGGCAGCGTCCCAAGCTCCTGATCTACCAGGCATCCAAGGTGCC
ATCTGGGGTCTCATCGCGGTTCAGCGGCAGTGGATCTGGGACACAGTTCATTCTC
ACCATCAGCGGCGTGCAGTGTGACGATGCTGCCACTTACTACTGTCAAGGCACTT
ATGATGGTAGTGGTTGGTCTAATGCTTTCGGCGGAGGGACCGAGGTGGTGGTCA
AA
16H2
Amino acid sequence (SEQ ID NO: 267):
QSVEESGGRLVTPGTPLTLTCTVSGIDLSSNAMTWVRQAPGEGLEWIGIISNSGSTYY
ASWAKGRFTISKTSSTTVDLKMTSLTTEDTATYFCARGFRYPNPWGPGTLVTVSS
Nucleic acid sequence (SEQ ID NO: 268):
CAGTCGGTGGAGGAGTCCGGGGGTCGCCTGGTCACGCCTGGGACACCCCTGACA
CTCACCTGCACAGTCTCTGGAATCGACCTCAGTAGCAATGCAATGACCTGGGTCC
GCCAGGCTCCAGGGGAGGGGCTGGAATGGATCGGAATCATTAGTAATAGTGGTA
GCACATACTACGCGAGCTGGGCGAAAGGCCGATTCACCATCTCCAAAACCTCGT
CGACCACGGTGGATCTGAAAATGACCAGTCTGACAACCGAGGACACGGCCACCT
ATTTCTGTGCCAGAGGTTTTAGATATCCTAATCCCTGGGGCCCAGGCACCCTGGT
CACCGTCTCCTCA
16L1
Amino acid sequence (SEQ ID NO: 269):
QVLTQTPSSVSAAVGGTVTINCQASESVGNNNYLSWYQQRPGQPPKVLIYDASRLAS
GVSSRFKGSGSGTQFTLTISGVQCDDAATYYCLGGYVSSGWYGAFGGGTEVVVK
Nucleic acid sequence (SEQ ID NO: 270):
CAAGTGCTGACCCAGACTCCATCCTCCGTGTCTGCAGCTGTGGGAGGCACAGTCA
CCATCAATTGCCAGGCCAGTGAGAGTGTTGGTAATAACAACTACTTATCCTGGTA
TCAGCAAAGACCAGGGCAGCCTCCCAAGGTCTTGATCTACGATGCATCCAGGCT
GGCATCTGGGGTCTCATCGCGGTTCAAAGGCAGTGGATCTGGGACACAGTTCACT
CTCACCATCAGTGGTGTGCAATGTGACGATGCTGCCACTTACTATTGTCTAGGCG
GTTATGTTAGTAGTGGTTGGTATGGGGCTTTCGGCGGAGGGACCGAGGTGGTGGT
CAAA
17H1
Amino acid sequence (SEQ ID NO: 271):
QSLEESGGDLVKPGASLILTCTASGFDFSSNALCWVRQAPGKGLEWIASIYAGGDTY
YATWAKGRFTVSKTSSTTVFLQMTSLTAADTATYFCARGAMTYSLWGPGTLVTVSS
Nucleic acid sequence (SEQ ID NO: 272):
CAGTCGTTGGAGGAGTCCGGGGGAGACCTGGTCAAGCCTGGGGCATCCCTGATA
CTCACCTGCACAGCCTCTGGATTCGACTTCAGTAGCAATGCACTGTGCTGGGTCC
GCCAGGCTCCAGGGAAGGGGCTGGAGTGGATCGCATCCATTTATGCTGGTGGTG
ACACTTACTACGCGACCTGGGCGAAAGGCCGATTCACCGTCTCCAAAACCTCGTC
GACCACGGTGTTTCTGCAGATGACCAGTCTGACAGCCGCGGACACGGCCACCTAT
TTCTGTGCGAGGGGTGCTATGACTTATAGTTTGTGGGGCCCAGGCACCCTGGTCA
CCGTCTCCTCA
17L1
Amino acid sequence (SEQ ID NO: 273):
ADIVMTQTPASVEAAMGGTVTINCQASQSVHNNNYLSWYQQKPGQPPKLLIYQASK
LASGGPSRFKGSGSGTEFTLTISDLECADAATYYCQSYYYSGSSGAVNSFGGGTEVV
VK
Nucleic acid sequence (SEQ ID NO: 274):
GCTGACATTGTGATGACCCAGACTCCAGCCTCCGTGGAGGCAGCTATGGGAGGC
ACAGTCACCATCAACTGCCAGGCCAGTCAGAGTGTTCATAATAATAACTACTTAT
CCTGGTATCAGCAGAAACCAGGGCAGCCTCCCAAGCTCCTGATCTACCAGGCATC
CAAACTGGCATCTGGGGGCCCATCGCGGTTCAAAGGCAGTGGATCTGGGACAGA
GTTCACTCTCACCATCAGCGACCTGGAGTGTGCCGATGCTGCCACTTACTACTGT
CAAAGCTATTATTATAGTGGTAGTAGTGGTGCCGTTAATTCTTTCGGCGGAGGGA
CCGAGGTGGTGGTCAAA
24H1
Amino acid sequence (SEQ ID NO: 275):
QEQLKESGGDLVTPGTPLTLTCTVSGFSLSSNAISWVRQAPGKGLEWIGVIYAGGGA
FYANWAKGRFTFSKTSTTVDLKMTSLTTEDTASYFCTRGYTYLAFWGQGTLVTVSS
Nucleic acid sequence (SEQ ID NO: 276):
CAGGAGCAGTTGAAGGAGTCCGGGGGAGACCTGGTCACGCCTGGGACACCCCTG
ACACTCACCTGCACAGTCTCTGGATTCTCCCTCAGTAGCAATGCAATAAGCTGGG
TCCGCCAGGCTCCAGGGAAGGGGCTGGAATGGATCGGAGTCATTTATGCTGGTG
GTGGCGCATTCTACGCGAACTGGGCGAAAGGCCGATTCACCTTCTCCAAAACCTC
GACCACGGTGGATCTGAAAATGACCAGTCTGACAACCGAGGACACGGCCTCTTA
TTTCTGTACCAGAGGCTATACTTATTTGGCCTTCTGGGGCCAGGGCACCCTGGTC
ACCGTCTCCTCA
24L1
Amino acid sequence (SEQ ID NO: 277):
ADIVMTQTPASVEAAVGGTVTINCQASQSISNLISWYQQKPGQPPKWYKASTLASG
VSSRFKGSGSGTEYTLTISDLECADAATYYCQGSAYGTSDVCAFGGGTEVVVK
Nucleic acid sequence (SEQ ID NO: 278):
GCTGACATTGTGATGACCCAGACTCCAGCCTCCGTGGAGGCAGCTGTGGGAGGC
ACAGTCACCATCAATTGCCAGGCCAGTCAGAGCATTAGCAACCTCATTTCTTGGT
ATCAGCAGAAACCAGGGCAGCCTCCCAAACTCCTGATCTACAAGGCATCCACTCT
GGCATCTGGGGTCTCATCGCGGTTCAAAGGCAGTGGATCTGGGACAGAGTACAC
TCTCACCATCAGCGACCTGGAGTGTGCCGATGCTGCCACTTACTACTGTCAAGGC
TCTGCTTATGGTACTAGTGATGTTTGTGCTTTCGGCGGAGGGACCGAGGTGGTGG
TCAAA
26H1
Amino acid sequence (SEQ ID NO: 279):
QSVEESGGRLVTPGTPLTLTCTVSGIDLSSNAMTWVRQAPGEGLEWIGAIDANGSPY
YTNWAKGRFTISKTSTTVTLKMTSPTTEDTATYFCARGYTRLDLWGQGTLVTVSS
Nucleic acid sequence (SEQ ID NO: 280):
CAGTCGGTGGAGGAGTCCGGGGGTCGCCTGGTCACGCCTGGGACACCCCTGACA
CTCACCTGCACAGTCTCTGGAATCGACCTCAGTAGCAATGCAATGACCTGGGTCC
GCCAGGCTCCAGGGGAGGGGCTGGAATGGATCGGAGCCATTGATGCTAATGGTA
GCCCATACTACACGAACTGGGCGAAAGGCCGATTCACCATCTCCAAAACCTCGA
CCACGGTGACTCTGAAAATGACCAGTCCGACAACCGAGGACACGGCCACCTATT
TCTGTGCCAGAGGGTATACTCGGTTGGATCTCTGGGGCCAGGGCACCCTGGTCAC
CGTCTCCTCA
26L1
Amino acid sequence (SEQ ID NO: 281):
QVLTQTPSSVSAAVGGTVTINCQSSQSILSDNYLAWYQQKPGQPPKLLIYQASKLVSG
VSSRFKGSGSGTGFTLTISGVQCDDAATYYCQGAYDSSDWYGAFGGGTEVVVK
Nucleic acid sequence (SEQ ID NO: 282):
CAAGTGCTGACCCAGACTCCATCCTCCGTGTCTGCAGCTGTGGGAGGCACAGTCA
CCATCAATTGCCAGTCCAGTCAGAGTATTTTGAGTGACAACTACTTAGCCTGGTA
TCAGCAGAAACCAGGGCAGCCTCCCAAGCTCCTGATCTACCAGGCATCCAAATT
GGTTTCTGGGGTCTCATCGCGATTCAAAGGCAGTGGATCTGGGACAGGATTCACT
CTCACCATCAGCGGCGTGCAGTGTGACGATGCTGCCACTTACTACTGTCAAGGCG
CTTATGATAGTAGTGATTGGTACGGTGCTTTCGGCGGAGGGACCGAGGTGGTGGT
CAAA
27H1
Amino acid sequence (SEQ ID NO: 283):
QSVEESGGRLVTPGTPLTLTCTVSGFSLNNYAMIWVRQAPGEGLEYIGFINSGGSAYY
ASWAKGRFTISRTSTTVDLKMTSLTAADTATYFCARGVPKMDLWGQGTLVTVSS
Nucleic acid sequence (SEQ ID NO: 284):
CAGTCGGTGGAGGAGTCCGGGGGTCGCCTGGTCACGCCTGGGACACCCCTGACA
CTCACCTGCACCGTCTCTGGATTCTCCCTCAATAACTATGCAATGATCTGGGTCCG
CCAGGCTCCAGGGGAGGGGCTGGAATACATCGGATTCATTAATTCTGGTGGTAG
CGCATACTACGCGAGCTGGGCAAAAGGCCGATTCACCATCTCCAGAACCTCGAC
CACGGTGGATCTGAAAATGACCAGTCTGACAGCCGCGGACACGGCCACCTATTT
CTGTGCCAGAGGGGTTCCTAAGATGGACTTGTGGGGCCAAGGCACCCTGGTCAC
CGTCTCCTCA
27L2
Amino acid sequence (SEQ ID NO: 285):
IVMTQTPSSVSAAVGGTVTINCQASQSVYNGNELSWYQQKPGQPPKLLIYAASILAS
GVPSRFKGSGWGTHFTLTISDVVCDDAATYYCAGYQSSVIDDIGFGGGTEVVVK
Nucleic acid sequence (SEQ ID NO: 286):
ATCGTGATGACCCAGACTCCATCCTCCGTGTCTGCAGCTGTGGGAGGCACAGTCA
CCATCAATTGCCAGGCCAGTCAAAGTGTTTATAATGGCAACGAATTATCCTGGTA
TCAGCAGAAACCAGGGCAGCCTCCCAAGCTCCTGATCTATGCTGCATCCATTTTG
GCATCCGGGGTCCCATCGCGGTTCAAAGGCAGTGGGTGGGGGACACACTTCACT
CTCACCATCAGCGATGTGGTGTGCGACGATGCTGCCACTTACTACTGTGCAGGAT
ATCAAAGTAGCGTTATTGATGATATTGGTTTCGGCGGAGGGACCGAGGTGGTGGT
CAAA
31H2
Amino acid sequence (SEQ ID NO: 287):
QSVEESGGRLVTPGGSLTLTCTVSGFSLNNYAMIWVRQAPGEGLEYIGFINTGDRAY
YASWAKGRFTISKTSSTTVDLKMTSLTAADTATYFCARGVPAMGLWGQGTLVTVSS
Nucleic acid sequence (SEQ ID NO: 288):
CAGTCGGTGGAGGAGTCCGGGGGTCGCCTGGTAACGCCTGGAGGATCCCTGACA
CTCACCTGCACAGTCTCTGGATTCTCCCTCAATAACTATGCAATGATCTGGGTCC
GCCAGGCTCCAGGGGAGGGACTGGAATACATCGGATTCATTAATACTGGTGATC
GCGCATACTATGCGAGCTGGGCAAAAGGCCGATTCACCATCTCCAAAACCTCGTC
GACCACGGTGGATCTGAAAATGACCAGTCTGACAGCCGCGGACACGGCCACCTA
TTTCTGTGCCAGAGGGGTTCCTGCTATGGGCTTGTGGGGCCAGGGCACCCTGGTC
ACCGTCTCCTCA
31L1
Amino acid sequence (SEQ ID NO: 289):
IVMTQTPSPVSAAVGDPVTINCQASQSVYNNNELSWYQQKPGQAPKLLIYAASYVAS
GVPSRFKGSGSGTQFTLTISNVVCDDAATYYCAGYESSGIDDIGFGGGTEVVVK
Nucleic acid sequence (SEQ ID NO: 290):
ATCGTGATGACCCAGACTCCATCTCCCGTGTCTGCAGCTGTGGGAGATCCAGTCA
CCATCAATTGCCAGGCCAGTCAGAGTGTTTATAATAATAACGAATTATCCTGGTA
TCAGCAGAAACCTGGGCAGGCTCCCAAGCTCCTGATCTATGCTGCATCCTATGTG
GCATCTGGGGTCCCATCGCGGTTCAAAGGCAGTGGATCTGGGACGCAGTTCACTC
TCACCATCAGCAATGTGGTGTGTGACGATGCTGCCACTTACTACTGTGCAGGATA
TGAAAGTAGTGGTATTGATGATATTGGTTTCGGCGGAGGGACCGAGGTGGTGGT
CAAA
45H1
Amino acid sequence (SEQ ID NO: 291):
QSVEESGGRLVTPGTPLTLTCTVSGFSLSSNAMTWVRQAPGQGLEWIGIIYASGSTYY
ASWAKGRFTISKTSSTTVDLKMTSPTTEDTATYFCARGFARLPLWGQGTLVTVSS
Nucleic acid sequence (SEQ ID NO: 292):
CAGTCGGTGGAGGAGTCCGGGGGTCGCCTGGTCACGCCTGGGACACCCCTGACA
CTCACCTGCACAGTCTCTGGATTCTCCCTCAGTAGCAATGCAATGACCTGGGTCC
GCCAGGCTCCAGGGCAGGGGCTGGAATGGATCGGAATCATTTATGCTAGTGGTA
GCACATACTACGCGAGCTGGGCGAAAGGCCGATTCACCATCTCCAAAACCTCGT
CGACCACGGTGGATCTGAAAATGACCAGTCCGACAACCGAGGACACGGCCACCT
ATTTCTGTGCCAGAGGATTTGCCCGGTTGCCGTTGTGGGGCCAGGGCACCCTGGT
CACCGTCTCCTCA
45L2
Amino acid sequence (SEQ ID NO: 293):
QVLTQTPSSVSAAVGGTVTINCQSSQSVGSNYLSWYQQKPGQPPKLLIYDASTLASG
VPSRFSGSGSGTQFTLTISGVQCDDAATYYCQGSYYSSDWYGAFGGGTEVVVK
Nucleic acid sequence (SEQ ID NO: 294):
CAAGTGCTGACCCAGACTCCATCCTCCGTGTCTGCAGCCGTGGGAGGCACAGTCA
CCATCAATTGCCAGTCCAGTCAGAGTGTTGGTAGTAACTACTTATCCTGGTATCA
GCAGAAACCAGGGCAGCCTCCCAAGCTCTTGATCTACGATGCATCCACTCTGGCA
TCTGGGGTCCCATCGCGGTTTAGCGGCAGTGGATCTGGGACACAGTTCACTCTCA
CCATCAGCGGCGTGCAGTGTGACGATGCTGCCACTTACTACTGTCAAGGCAGTTA
TTATAGTAGTGATTGGTACGGTGCTTTCGGCGGAGGGACCGAGGTGGTGGTCAA
A
58H2
Amino acid sequence (SEQ ID NO: 295):
QSLEESGGDLVKPGASLTLTCTATGFSFNTNYYMCWVRQAPGKGLELIACSYTTSGS
TYYATWAKGRFTFSKTSSTTVTLQMTSLTAADTATYFCVKYGAGYTYNLWGPGTL
VTVSS
Nucleic acid sequence (SEQ ID NO: 296):
CAGTCGTTGGAGGAGTCCGGGGGAGACCTGGTCAAGCCGGGGGCATCCCTGACA
CTCACCTGCACAGCCACTGGATTCTCCTTCAATACCAACTACTACATGTGCTGGG
TCCGCCAGGCTCCAGGGAAGGGGCTGGAGTTGATCGCATGCAGTTATACTACTA
GTGGTAGTACTTACTATGCGACCTGGGCGAAAGGCCGATTCACCTTCTCCAAAAC
CTCGTCGACCACGGTGACTCTGCAAATGACCAGTCTGACAGCCGCGGACACGGC
CACCTATTTCTGTGTGAAATATGGTGCTGGTTATACTTATAACTTGTGGGGCCCA
GGCACCCTGGTCACCGTCTCCTCA
58L1
Amino acid sequence (SEQ ID NO: 297):
ALVMTQTPSSVSAAVGGTVTIKCQASESISNYLAWYQQKPGQPPNLLIYRASTLESG
VPSRFKGSGSGTEFTLTISDLECADAATYFCQQGYSNTNLDNIFGGGTEVVVK
Nucleic acid sequence (SEQ ID NO: 298):
GCCCTTGTGATGACCCAGACTCCATCCTCCGTGTCTGCAGCTGTGGGAGGCACAG
TCACCATCAAGTGCCAGGCCAGTGAGAGCATTAGTAACTACTTAGCCTGGTATCA
GCAGAAACCAGGGCAGCCTCCCAATCTCCTGATCTACAGGGCATCCACTCTGGA
ATCTGGGGTCCCATCGCGGTTCAAAGGCAGTGGATCTGGGACAGAGTTCACTCTC
ACCATCAGCGACCTGGAGTGTGCCGATGCTGCCACTTACTTCTGTCAGCAGGGTT
ACAGTAATACTAATCTTGATAATATTTTCGGCGGAGGGACCGAGGTGGTGGTCAA
A
70H1
Amino acid sequence (SEQ ID NO: 299):
QSVEESGGRLVTPGTPLTLTCTVSGFSLSSNAISWVRQAPGEGLEYIGWIDATGSAYY
ATWAKGRFTISKTSSTTVDLKMTSPTTEDTATYFCARGFRYSAFWGQGTLVTVSS
Nucleic acid sequence (SEQ ID NO: 300):
CAGTCGGTGGAGGAGTCCGGGGGTCGCCTGGTCACGCCTGGGACACCCCTGACA
CTCACCTGCACCGTCTCTGGATTCTCCCTCAGTAGCAATGCAATAAGCTGGGTCC
GCCAGGCTCCAGGGGAGGGGCTGGAGTACATCGGATGGATTGATGCTACTGGTA
GCGCATACTACGCGACCTGGGCGAAAGGCCGATTCACCATCTCTAAAACCTCGTC
GACCACGGTGGATCTGAAGATGACCAGTCCGACAACCGAGGACACGGCCACCTA
TTTCTGTGCCAGAGGGTTTAGGTATTCTGCGTTCTGGGGCCAAGGCACCCTGGTC
ACCGTCTCCTCA
70L2
Amino acid sequence (SEQ ID NO: 301):
QVLTQTPSPVSVAVGGTVTINCQASQSVYNNNYLSWYQQKPGQPPKWYDTSTLAS
GIPSRFKGSGSGTQFTLTISDLECDDAATYYCAGTYSTSDWSVAFGGGTEVVV
Nucleic acid sequence (SEQ ID NO: 302):
CAAGTGCTGACCCAGACACCATCGCCCGTGTCTGTAGCTGTGGGAGGCACAGTC
ACCATCAATTGCCAGGCCAGTCAGAGTGTTTATAATAACAACTACTTATCCTGGT
ATCAACAGAAACCAGGGCAGCCTCCCAAGCTCCTGATCTATGATACATCCACTCT
GGCATCTGGGATCCCATCGCGGTTCAAAGGCAGTGGATCTGGGACACAGTTCACT
CTCACCATCAGCGACCTGGAGTGTGACGATGCTGCCACTTATTATTGTGCAGGCA
CTTATTCTACTAGTGATTGGTCTGTTGCTTTCGGCGGAGGGACCGAGGTGGTGGT
CAAA
78H2
Amino acid sequence (SEQ ID NO: 303):
QSVEESGGRLVTPGTPLTLTCTVSGIDLSTYFMSWVRQAPGKGLEYIGWINTNDKIYY
ASWAKGRFTISTTSTTVDLKITSPTTEDTATYFCGSPYPRYASGLNLWGQGTLVTVSS
Nucleic acid sequence (SEQ ID NO: 304):
CAGTCGGTGGAGGAGTCCGGGGGTCGCCTGGTCACGCCTGGGACACCCCTGACA
CTCACCTGCACAGTCTCTGGAATCGACCTCAGTACCTATTTCATGAGCTGGGTCC
GCCAGGCTCCAGGGAAGGGGCTGGAATACATCGGGTGGATTAATACTAATGATA
AAATATACTACGCGAGCTGGGCGAAGGGCCGATTCACCATCTCCACAACCTCGA
CCACGGTGGATCTGAAAATCACCAGTCCGACAACCGAGGACACGGCCACCTATT
TCTGTGGCAGTCCTTATCCTAGGTATGCTAGTGGTCTTAACTTGTGGGGCCAAGG
CACCCTGGTCACCGTCTCCTCA
78L1
Amino acid sequence (SEQ ID NO: 305):
ADVVMTQTPASVSEPVGGTVTIKCQASQSIHNYLAWYQQKPGQPPKWYSASNLAS
GVSSRFKGSGSGTEYTLTISDLECADAATYYCQCTYYGSSYENTFGGGTEVVVK
Nucleic acid sequence (SEQ ID NO: 306):
GCCGATGTTGTGATGACCCAGACTCCAGCCTCCGTGTCTGAACCTGTGGGAGGCA
CAGTCACCATCAAGTGCCAGGCCAGTCAGAGCATTCATAATTACTTAGCCTGGTA
TCAGCAGAAACCAGGGCAGCCTCCCAAGCTCCTGATCTATTCTGCATCCAATCTG
GCATCTGGGGTCTCATCGCGGTTCAAAGGCAGTGGATCTGGGACAGAATACACT
CTCACCATCAGCGACCTGGAGTGTGCCGATGCTGCCACTTACTACTGTCAATGTA
CTTATTATGGTAGTAGTTATGAGAATACTTTCGGCGGAGGGACCGAGGTGGTGGT
CAAA
86H2
Amino acid sequence (SEQ ID NO: 307):
QSLEESGGRLVTPGGSLTLTCTVSGIDLSRYHMSWVRQAPGKGLEWIATTHIDGGVY
YAIWAKGRFTISKTSTTVDLKMTSLTAEDTATYFCARKFDLWGQGTLVTVSS
Nucleic acid sequence (SEQ ID NO: 308):
CAGTCGCTGGAGGAGTCCGGGGGTCGCCTGGTAACGCCTGGAGGATCCCTGACA
CTCACCTGCACAGTCTCTGGAATCGACCTCAGTAGGTACCACATGAGCTGGGTCC
GCCAGGCTCCAGGGAAGGGGCTGGAATGGATCGCAACGACTCATATTGATGGTG
GCGTATACTACGCGATTTGGGCGAAAGGCCGATTCACCATCTCCAAAACCTCGAC
CACGGTGGATCTGAAAATGACCAGTCTGACAGCCGAGGACACGGCCACCTATTT
CTGTGCCAGAAAGTTTGACTTGTGGGGCCAAGGCACCCTGGTCACCGTCTCCTCA
G
86L2
Amino acid sequence (SEQ ID NO: 309):
QVLTQTPSSVSAAVGGTVTISCQSSESVSNNNWLSWYQQKSGQPPKLLIYQASKLAS
GVSSRFKGSGSGTQFTLTISDLECADAATYYCQGGYYDSGWYYAFGGGTEVVVK
Nucleic acid sequence (SEQ ID NO: 310):
CAAGTGCTGACCCAGACTCCATCGTCCGTGTCTGCAGCTGTGGGAGGCACAGTCA
CCATCAGTTGCCAGTCCAGTGAGAGCGTTTCAAATAATAACTGGTTATCCTGGTA
TCAGCAGAAATCAGGGCAGCCTCCCAAGCTCCTGATCTACCAGGCATCCAAACT
GGCATCTGGGGTCTCATCGCGGTTCAAAGGCAGTGGATCTGGGACACAGTTCACT
CTCACCATCAGCGACCTGGAGTGTGCCGATGCTGCCACTTACTACTGTCAAGGCG
GTTATTATGATAGTGGTTGGTACTATGCTTTCGGCGGAGGGACCGAGGTGGTGGT
CAAA
91H1
Amino acid sequence (SEQ ID NO: 311):
QSVEESGGRLVTPGTPLTLTCTVSGFSLSSNAISWVRQAPGKGLEWIGFIDSEGSASY
ASWANGRFTISKTSNTVDLKMTGLTTEDTATYFCARGFRYLPLWGQGTLVTVSS
Nucleic acid sequence (SEQ ID NO: 312):
CAGTCGGTGGAGGAGTCCGGGGGTCGCCTGGTCACGCCTGGGACACCCCTGACA
CTCACCTGCACCGTCTCTGGATTCTCCCTCAGTAGCAATGCAATAAGCTGGGTCC
GCCAGGCTCCAGGGAAGGGGCTGGAGTGGATCGGATTCATTGATAGTGAGGGTA
GCGCATCCTACGCGAGCTGGGCGAATGGTCGATTCACCATCTCCAAAACCTCGAA
CACGGTGGATCTGAAAATGACCGGTCTGACAACCGAGGACACGGCCACCTATTT
CTGTGCCAGAGGATTTCGGTACTTGCCCTTGTGGGGCCAAGGCACCCTGGTCACC
GTCTCCTCA
91L1
Amino acid sequence (SEQ ID NO: 313):
AVLTQTPSPVSAAVGGTVTINCQASQSVYYNNYLAWYQQKPGQPPKWYDTSKLAS
GVPSRFKGSGSGTQFTLTISGVQCDDAASYFCQGTYYSSGWWNAFGGGTEVVVK
Nucleic acid sequence (SEQ ID NO: 314):
GCCGTGCTGACCCAGACACCATCCCCCGTGTCTGCAGCTGTGGGAGGCACAGTCA
CCATCAATTGCCAGGCCAGTCAGAGTGTTTATTATAACAACTACTTAGCCTGGTA
TCAGCAGAAACCAGGGCAGCCTCCCAAACTCCTGATCTACGATACATCCAAATTG
GCATCTGGGGTCCCATCCCGGTTCAAAGGCAGTGGATCTGGGACACAGTTCACTC
TCACCATCAGCGGCGTGCAGTGTGACGATGCTGCCTCTTACTTCTGTCAAGGCAC
TTATTATAGTAGTGGTTGGTACTGGAATGCTTTCGGCGGAGGGACCGAGGTGGTG
GTCAAA
93H2
Amino acid sequence (SEQ ID NO: 315):
QEQLKESGGDLVTPGTPLTLTCTVSGFSLSSNAISWVRQAPGKGLEWIGVIYAGGGA
FYASWAKGRFTFSKTSTTVDLKMTSLTTEDTASYFCTRGYTYLAFWGQGTLVTVSS
Nucleic acid sequence (SEQ ID NO: 316):
CAGGAGCAACTGAAGGAGTCCGGGGGAGACCTGGTCACGCCTGGGACACCCCTG
ACACTCACCTGCACAGTCTCTGGGTTCTCCCTCAGTAGCAATGCAATAAGCTGGG
TCCGCCAGGCTCCAGGGAAGGGGCTGGAATGGATCGGAGTCATTTATGCTGGTG
GTGGCGCATTCTACGCGAGCTGGGCGAAAGGCCGATTCACCTTCTCCAAAACCTC
GACCACGGTGGATCTGAAAATGACCAGTCTGACAACCGAGGACACGGCCTCCTA
TTTCTGTACCAGAGGCTATACTTATTTGGCCTTCTGGGGCCAGGGCACCCTGGTC
ACCGTCTCCTCA
93L2
Amino acid sequence (SEQ ID NO: 317):
DVVMTQTPASVSEPVGGTVTIRCQASQSINNFLSWYQQKPGQPPKWYKASTLASG
VSSRFKGSGSGTEYTLTISDLECADAATYYCQGSAYGTSDVCAFGGGTEVVVK
Nucleic acid sequence (SEQ ID NO: 318):
GATGTTGTGATGACCCAGACTCCAGCCTCCGTGTCTGAACCTGTGGGAGGCACAG
TCACCATCAGGTGCCAGGCCAGTCAGAGCATTAACAATTTCTTATCTTGGTATCA
GCAGAAACCAGGGCAGCCTCCCAAGCTCCTGATCTACAAGGCATCCACTCTGGC
ATCTGGGGTCTCATCGCGGTTCAAAGGCAGTGGATCTGGGACAGAGTACACTCTC
ACCATCAGCGACCTGGAGTGTGCCGATGCTGCCACTTACTACTGTCAAGGCTCTG
CTTATGGTACTAGTGATGTTTGTGCTTTCGGCGGAGGGACCGAGGTGGTGGTCAA
A
94H1
Amino acid sequence (SEQ ID NO: 319):
QSLEESGGDLVKPGASLILTCTASGFDFSSNALCWVRQAPGKGLEWIASIYAGGDTY
YATWAKGRFTVSKTSSTTVFLQMTSLTAADTATYFCARGAMTYSLWGPGTLVTVSS
Nucleic acid sequence (SEQ ID NO: 320):
CAGTCGTTGGAGGAGTCCGGGGGAGACCTGGTCAAGCCTGGGGCATCCCTGATA
CTCACCTGCACAGCCTCTGGATTCGACTTCAGTAGCAATGCACTGTGCTGGGTCC
GCCAGGCTCCAGGGAAGGGGCTGGAGTGGATCGCATCCATTTATGCTGGTGGTG
ACACTTACTACGCGACCTGGGCGAAAGGCCGATTCACCGTCTCCAAAACCTCGTC
GACCACGGTGTTTCTGCAGATGACCAGTCTGACAGCCGCGGACACGGCCACCTAT
TTCTGTGCGAGGGGTGCTATGACTTATAGTTTGTGGGGCCCAGGCACCCTGGTCA
CCGTCTCCTCA
94L2
Amino acid sequence (SEQ ID NO: 321):
ADIVMTQTPASVEAAVGGTVTINCQASQSVHNNNYLSWYQQKPGQPPKLLIYQASK
LASGGPSRFKGSGSGTEFTLTISDLECADAATYYCQSYYYSGCSGAVNSFGGGTEVV
VK
Nucleic acid sequence (SEQ ID NO: 322):
GCTGACATTGTGATGACCCAGACTCCAGCCTCCGTGGAGGCAGCTGTGGGAGGC
ACAGTCACCATCAACTGCCAGGCCAGTCAGAGTGTTCATAATAATAACTACTTAT
CCTGGTATCAGCAGAAACCAGGGCAGCCTCCCAAGCTCCTGATCTACCAGGCATC
CAAACTGGCATCTGGGGGCCCATCGCGGTTCAAAGGCAGTGGATCTGGGACAGA
GTTCACTCTCACCATCAGCGACCTGGAGTGTGCCGATGCTGCCACTTACTACTGT
CAAAGCTATTATTATAGTGGTTGTAGTGGTGCCGTTAATTCTTTCGGCGGAGGGA
CCGAGGTGGTGGTCAAA
102H1
Amino acid sequence (SEQ ID NO: 323):
QSLEESGGRLVTPGGSLTLTCTVSGIDLSSNAVGWVRQAPGKGLEYIGVISVSGNIYY
ANWARGRFTISKTSSTTVDLKMTSLTAADTATYFCARPWDLWGQGTLVTVSS
Nucleic acid sequence (SEQ ID NO: 324):
CAGTCGCTGGAGGAGTCCGGGGGTCGCCTGGTAACGCCTGGAGGATCCCTGACA
CTCACCTGCACAGTCTCTGGAATCGACCTCAGTAGCAATGCAGTGGGCTGGGTCC
GCCAGGCTCCAGGGAAGGGGCTGGAATACATCGGAGTCATTAGTGTTAGTGGTA
ACATATACTACGCGAACTGGGCGAGAGGCCGATTCACCATCTCCAAAACCTCGTC
GACCACGGTGGATCTGAAAATGACCAGTCTGACAGCCGCGGACACGGCCACCTA
TTTCTGTGCCAGACCCTGGGACTTGTGGGGCCAAGGCACCCTGGTCACCGTCTCC
TCA
102L1
Amino acid sequence (SEQ ID NO: 325):
DVVMTQTPASVSEAVGGTVTIKCQASESISSWLAWYQQKPGQPPKWYLASTLASG
VPSRFKGSGSGTQFTLTISDLECADAATYYCQCSSYTSGYVAAFGGGTEVVVK
Nucleic acid sequence (SEQ ID NO: 326):
GATGTTGTGATGACCCAGACTCCAGCCTCCGTGTCTGAAGCTGTGGGAGGCACAG
TCACCATCAAGTGCCAGGCCAGTGAGAGCATTAGCAGTTGGTTAGCCTGGTATCA
GCAGAAACCAGGGCAGCCTCCCAAGCTCCTGATCTATCTGGCATCTACTCTGGCA
TCTGGGGTCCCATCGCGGTTCAAAGGCAGTGGATCTGGGACACAGTTCACTCTCA
CCATCAGCGACCTGGAGTGTGCCGATGCTGCCACTTACTACTGTCAATGTTCTTCT
TATACGAGTGGTTATGTTGCCGCTTTCGGCGGAGGGACCGAGGTGGTGGTCAAA
103H2
Amino acid sequence (SEQ ID NO: 327):
QSMEESGGRLVTPGTPLTLTCTVSTFSLNSFHMSWVRQAPGKGLEWIGVIHPNDATY
YASWAKGRFTISKTSTTVDLKITSPTTEDTATYFCARDLAGYSTGGSFWGQGTLVTV
SS
Nucleic acid sequence (SEQ ID NO: 328):
CAGTCAATGGAGGAGTCCGGGGGTCGCCTGGTCACGCCTGGGACACCCCTGACA
CTCACCTGCACAGTCTCTACATTCTCCCTCAATAGTTTCCACATGAGCTGGGTCCG
CCAGGCTCCAGGGAAGGGGCTGGAATGGATCGGCGTCATTCATCCTAATGATGC
CACATACTACGCGAGCTGGGCGAAAGGCCGATTCACCATCTCCAAAACCTCGAC
CACGGTGGATCTGAAAATCACCAGTCCGACAACCGAGGACACGGCCACCTATTT
CTGTGCCAGAGATCTTGCTGGTTATAGTACTGGTGGTAGCTTCTGGGGCCAAGGC
ACCCTGGTCACCGTCTCCTCA
103L2
Amino acid sequence (SEQ ID NO: 329):
ALVLTQTPSPVSAAVGGTVTVSCQASQSVYNNNWLSWFQQKPGQPPKLLIYRASTL
ASGVPSRFSGSGSGTQFTLTISGVQCADAATYYCAAYKSWSNDDFGFGGGTEVVVK
Nucleic acid sequence (SEQ ID NO: 330):
GCGCTTGTGCTGACCCAGACTCCATCTCCCGTGTCTGCAGCTGTGGGAGGCACAG
TCACCGTCAGTTGCCAGGCCAGTCAGAGTGTTTATAATAACAACTGGTTATCCTG
GTTTCAGCAGAAACCAGGGCAGCCTCCCAAGCTCCTGATCTACAGGGCATCCACT
CTGGCATCTGGGGTCCCATCACGGTTCAGCGGCAGTGGATCTGGGACACAGTTCA
CTCTCACCATCAGTGGCGTGCAGTGTGCCGATGCTGCCACTTACTACTGTGCAGC
GTATAAAAGTTGGAGTAATGATGATTTTGGTTTCGGCGGAGGGACCGAGGTAGT
AGTCAAA
105H1
Amino acid sequence (SEQ ID NO: 331):
QSLEESGGRLVTPGTPLTITCTVSGIDLSSVAMGWVRQAPGKGLEYIGVISTSGNKYY
ATWAKGRFTISKTSTTVELKVTSPTTEDTATYFCARAWNLWGQGTLVTVSS
Nucleic acid sequence (SEQ ID NO: 332):
CAGTCGCTGGAGGAGTCCGGGGGTCGCCTGGTCACGCCTGGGACACCCCTGACA
ATCACCTGCACGGTCTCTGGAATCGACCTCAGTAGCGTTGCAATGGGCTGGGTCC
GCCAGGCTCCAGGGAAGGGGCTGGAATACATCGGAGTCATTAGTACTAGTGGTA
ATAAATACTACGCGACCTGGGCGAAAGGCCGATTCACCATCTCCAAAACCTCGA
CCACGGTGGAGCTGAAGGTCACCAGTCCGACAACCGAGGACACGGCCACCTATT
TCTGTGCCAGAGCCTGGAACTTGTGGGGCCAAGGCACCCTGGTCACCGTCTCCTC
A
105L4
Amino acid sequence (SEQ ID NO: 333):
DVVMTQTPASVSEPVGGTVTIKCRASEDIESYLAWYRQKPGQPPKWYRASKLASG
VPSRFSGSGSGTEYTLTISDLECADAATYYCQCTTYTSTYVGGGFGGGTEVVVK
Nucleic acid sequence (SEQ ID NO: 334):
GATGTAGTGATGACCCAGACTCCAGCCTCCGTGTCTGAACCTGTGGGAGGCACA
GTCACCATCAAGTGCCGGGCCAGTGAGGACATTGAAAGCTATTTAGCCTGGTATC
GGCAGAAACCAGGGCAGCCTCCCAAGCTCCTGATCTACAGGGCATCCAAACTGG
CATCTGGGGTCCCATCGCGGTTCAGTGGCAGTGGATCTGGGACAGAGTACACTCT
CACCATCAGCGACCTGGAGTGTGCCGATGCTGCCACTTACTACTGTCAATGTACT
ACTTATACGAGTACTTATGTTGGTGGTGGTTTCGGCGGAGGGACCGAGGTGGTGG
TCAAA
108H1
Amino acid sequence (SEQ ID NO: 335):
QSLEGSGGRLVKPDETLTITCTVSGFSLSSNAMSWVRQAPGKGLEWIGFIDAGGSAY
YATWVNGRFTISKTSTTVDLKMTSLTTEDTATYFCAKGLSWSDFWGQGTLVTVSS
Nucleic acid sequence (SEQ ID NO: 336):
CAGTCGCTGGAGGGGTCCGGGGGTCGCCTGGTCAAGCCTGACGAAACCCTGACA
ATCACCTGCACAGTCTCTGGATTCTCCCTCAGTAGCAATGCAATGAGCTGGGTCC
GCCAGGCTCCAGGGAAGGGGCTGGAATGGATCGGATTCATAGATGCGGGTGGTA
GCGCATACTACGCGACCTGGGTGAATGGCCGATTCACCATCTCCAAAACCTCGAC
CACGGTGGATCTGAAAATGACCAGTCTGACAACCGAGGACACGGCCACCTATTT
CTGTGCCAAAGGACTAAGCTGGTCTGACTTTTGGGGCCAGGGCACCCTGGTCACC
GTCTCCTCA
108L3
Amino acid sequence (SEQ ID NO: 337):
QVLTQTPSSVSAAVGGTVTVSCQSSQSVVSNNRLSWYQQKSGQPPKLLIYLASTLPS
GVPSRFRGSGSGTQFTLTISDLGCDDAATYYCQGTYGSSSYYGAFGGGTEVVVK
Nucleic acid sequence (SEQ ID NO: 338):
CAAGTGCTGACCCAGACTCCATCGTCCGTGTCTGCAGCTGTGGGAGGCACAGTCA
CCGTCAGTTGCCAGTCCAGTCAGAGTGTTGTTAGTAACAACCGCTTATCCTGGTA
TCAGCAGAAATCAGGGCAGCCTCCCAAGCTCCTGATCTATCTGGCATCCACTCTG
CCATCTGGGGTCCCATCGCGGTTCAGGGGCAGTGGATCTGGGACACAGTTCACTC
TCACCATCAGCGACCTGGGCTGTGACGATGCTGCCACTTACTACTGTCAAGGCAC
TTATGGTAGTAGTAGTTATTACGGAGCTTTCGGCGGAGGGACCGAGGTGGTGGTC
AAA
109H2
Amino acid sequence (SEQ ID NO: 339):
QSLEESGGRLVTPGTPLTLTCTASEFTISSFHMSWVRQAPGKGLEWIGVIHPNDITYY
ASWAKGRFTISKTSTTVELKITSPTTEDTATYFCVRDLTGGTTGGRLWGPGTLVTVSS
Nucleic acid sequence (SEQ ID NO: 340):
CAGTCGCTGGAGGAGTCCGGGGGTCGCCTAGTCACGCCTGGGACACCCCTGACA
CTCACCTGCACAGCCTCTGAATTTACCATTAGTAGCTTCCACATGAGCTGGGTCC
GCCAGGCTCCAGGGAAGGGGCTGGAATGGATCGGAGTCATTCATCCCAATGATA
TCACATATTACGCGAGCTGGGCGAAAGGCCGATTCACCATCTCCAAAACCTCGAC
CACGGTGGAGCTGAAGATCACCAGTCCGACAACCGAGGACACGGCCACCTATTT
CTGTGTCAGAGATCTTACTGGTGGTACTACTGGTGGTAGGTTGTGGGGCCCAGGC
ACCCTGGTCACCGTCTCCTCA
109L1
Amino acid sequence (SEQ ID NO: 341):
ALVLTQTPSPVSAAVGDTVTVSCQSSKSVCNNDWLSWFQQKPGQPPKLLIYRASTLA
SGVPSRFKGSGSGTQFTLTISGVECDDAATYYCAGYASWNNDDFGFGGETEVVVK
Nucleic acid sequence (SEQ ID NO: 342):
GCCCTTGTGCTGACCCAGACTCCATCCCCCGTGTCTGCAGCTGTGGGAGACACAG
TCACCGTCAGTTGCCAGTCCAGTAAGAGTGTTTGTAATAACGACTGGTTATCCTG
GTTTCAGCAGAAACCAGGGCAGCCTCCCAAGCTCCTGATCTACAGGGCATCCACT
CTGGCATCTGGGGTCCCATCTCGATTCAAAGGCAGTGGATCTGGGACACAATTCA
CTCTCACCATCAGCGGCGTGGAATGTGACGATGCTGCCACTTACTACTGTGCAGG
CTATGCAAGTTGGAATAATGATGATTTTGGTTTCGGCGGAGAGACCGAGGTGGTG
GTCAAA
110H1
Amino acid sequence (SEQ ID NO: 343):
QSVEESGGRLVTPGTSLTLTCTASGFSLSSWMGWVRQAPEKGLEYIGIISTSENIYY
ATWAKGRFTISKTSSTTVDLKITSPTTEDTATYFCARWSDLWGQGTLVTVSS
Nucleic acid sequence (SEQ ID NO: 344):
CAGTCGGTGGAGGAGTCCGGGGGTCGCCTGGTCACGCCTGGGACATCCCTGACA
CTCACCTGCACAGCCTCCGGATTCTCCCTCAGTAGCTACTGGATGGGCTGGGTCC
GCCAGGCTCCAGAGAAGGGGCTGGAATACATCGGAATCATTAGTACGAGTGAGA
ACATATACTACGCGACCTGGGCGAAAGGCCGATTCACCATCTCCAAAACCTCGTC
GACCACAGTGGATCTGAAAATCACCAGTCCGACAACCGAGGACACGGCCACCTA
TTTCTGTGCCAGATGGAGTGACTTGTGGGGCCAAGGCACCCTGGTCACCGTCTCC
TCA
110L1
Amino acid sequence (SEQ ID NO: 345):
QVLTQTPASVSAAVGGTVTINCQSSQSVGSGNILSWYQQKPGQPPKLLIYQASKLAS
GVSSRFKGSGSGTQFTLIISDVQCDDGASYYCLGSYGCSSADCAAFGGGTEVVVK
Nucleic acid sequence (SEQ ID NO: 346):
CAAGTGCTGACCCAGACTCCAGCCTCCGTGTCTGCAGCTGTGGGAGGCACAGTCA
CCATCAACTGCCAGTCCAGTCAGAGTGTTGGTAGTGGCAATATCTTATCCTGGTA
TCAGCAGAAACCAGGGCAGCCTCCCAAGCTCCTGATCTACCAGGCATCCAAACT
GGCATCTGGGGTCTCATCGCGGTTCAAAGGCAGTGGATCTGGGACACAGTTCACT
CTCATCATCAGCGACGTGCAGTGTGACGATGGTGCCTCTTACTACTGTCTAGGCA
GTTATGGTTGTAGTAGTGCTGATTGTGCTGCTTTCGGCGGAGGGACCGAGGTGGT
GGTCAAA
114H2
Amino acid sequence (SEQ ID NO: 347):
QSVEVSGGRLVTPGTPLTLTCTVSGFSLSSNAISWVRQAPGKGLEWIGIIDSNGSTYY
ASWAKGRFTISKASTTVDLKITGPTTEDTATYFCGRGAIYPALWGQGTLVTVSS
Nucleic acid sequence (SEQ ID NO: 348):
CAGTCGGTGGAGGTGTCCGGGGGTCGCCTGGTCACGCCTGGGACACCCCTGACA
CTCACCTGCACAGTCTCTGGATTCTCCCTCAGTAGCAATGCAATAAGCTGGGTCC
GCCAGGCTCCAGGGAAGGGGCTGGAATGGATCGGAATCATTGATAGTAATGGTA
GCACATACTACGCGAGCTGGGCGAAAGGCCGATTCACCATCTCCAAAGCCTCGA
CCACGGTGGATCTGAAAATCACCGGTCCGACAACCGAGGACACGGCCACCTATT
TCTGTGGCAGAGGGGCGATTTATCCGGCTTTGTGGGGCCAAGGCACCCTGGTCAC
CGTCTCCTCA
114L1
Amino acid sequence (SEQ ID NO: 349):
AFELTQTPASVEAAVGGTVTIKCQASQSISSSYLSWYQQKPGQPPKWYKASTLASG
VPSRFKGSGSGTQFTLTISGVQCDDAATYYCLYAYFGGSTAEHTFGGGTEVVVK
Nucleic acid sequence (SEQ ID NO: 350):
GCATTCGAATTGACCCAGACTCCAGCCTCCGTGGAGGCAGCTGTGGGAGGCACA
GTTACCATCAAGTGCCAGGCCAGTCAGAGTATTAGTAGTAGCTACTTATCCTGGT
ATCAGCAAAAACCAGGGCAGCCTCCCAAGCTCCTGATCTACAAGGCTTCCACTCT
GGCATCTGGGGTCCCATCGCGGTTCAAAGGCAGTGGATCTGGGACACAGTTCACT
CTCACCATCAGTGGCGTGCAGTGTGACGATGCTGCCACTTACTACTGTCTATACG
CTTATTTTGGTGGTAGTACTGCTGAGCATACTTTCGGCGGAGGGACCGAGGTGGT
GGTCAAA
120H1
Amino acid sequence (SEQ ID NO: 351):
QSLEESGGRLVTPGTPLTLTCTVSGIDLSRYYMSWVRQAPGKGLEWIATTHIDGGVY
YANWAKGRFTISKTATTVDLKMTSLTAEDTATYFCARKFDLWGQGTLVTVSS
Nucleic acid sequence (SEQ ID NO: 352):
CAGTCGCTGGAGGAGTCCGGGGGTCGCCTGGTCACGCCTGGGACACCCCTGACA
CTCACCTGCACAGTCTCTGGAATCGACCTCAGTAGGTACTACATGAGCTGGGTCC
GCCAGGCTCCAGGGAAGGGGCTGGAATGGATCGCAACGACTCATATTGATGGTG
GCGTATATTACGCGAACTGGGCGAAAGGCCGATTCACCATCTCCAAAACCGCGA
CCACGGTGGATCTGAAAATGACCAGTCTGACAGCCGAGGACACGGCCACCTATT
TCTGTGCCAGAAAATTTGACTTGTGGGGCCAAGGCACCCTGGTCACCGTCTCCTC
A
120L1
Amino acid sequence (SEQ ID NO: 353):
QELTQTPSSVSAAVGGTVTISCQSSESVSNNNWLSWYQQKPGQPPKLLIYAASKLAS
GVPSRFTGSGSGTQFTLTISDLECADAATYYCQGGYYDSGWYYAFGGGTEVVVK
Nucleic acid sequence (SEQ ID NO: 354):
TGCAGCTGTGGGAGGCACAGTCACCATCAGTTGCCAGTCCAGTGAGAGCGTTTCA
AATAACAACTGGTTATCCTGGTACCAGCAGAAACCAGGGCAGCCTCCCAAGCTC
CTGATCTATGCTGCATCCAAGCTGGCAAGTGGGGTCCCATCGCGGTTCACCGGCA
GTGGGTCTGGGACACAGTTCACTCTCACCATCAGCGACCTGGAGTGTGCCGATGC
TGCCACTTACTACTGCCAAGGCGGTTATTATGATAGTGGTTGGTACTATGCTTTCG
GCGGAGGGACCGAGGTGGTGGTCAAA
121H2
Amino acid sequence (SEQ ID NO: 355):
QSAEESGGRLGTPGTPLTLTCTVSGFSLSSNAINWVRQAPGKGLEWIGIIDAPGSTYY
ASWAKGRFTISKTSTTADLKITSPTTEDTATYFCARNYAYFALWGPGTLVTVSS
Nucleic acid sequence (SEQ ID NO: 356):
CAGTCGGCGGAGGAGTCCGGGGGTCGCCTGGGCACGCCTGGGACACCCCTGACA
CTCACCTGTACAGTCTCTGGATTCTCCCTCAGTAGCAATGCAATAAACTGGGTCC
GCCAGGCTCCAGGGAAGGGGCTGGAATGGATCGGAATCATTGATGCTCCTGGTA
GCACATACTACGCGAGCTGGGCGAAAGGCCGATTCACCATCTCCAAAACCTCGA
CCACGGCGGATCTGAAAATCACCAGTCCGACAACCGAGGACACGGCCACCTATT
TCTGTGCCAGAAATTATGCCTACTTTGCCTTATGGGGCCCAGGCACCCTGGTCAC
CGTCTCCTCA
121L1
Amino acid sequence (SEQ ID NO: 357):
AFEMTQTPSSVSEPVGGTVTIKCQASESVGSNNRLSWYQQKPGQPPKLLIYEASKLPS
GVPSRFRGSGSGTQFTLTISDIQREDAATYYCLGWHASTDDGWAFGAGTNVGIE
Nucleic acid sequence (SEQ ID NO: 358):
GCATTCGAGATGACCCAGACTCCATCCTCCGTGTCTGAACCTGTGGGAGGCACAG
TCACCATCAAGTGCCAGGCCAGTGAGAGTGTTGGTAGTAACAACCGCTTATCCTG
GTATCAGCAGAAACCAGGGCAGCCTCCCAAGCTCCTGATCTATGAAGCATCCAA
ACTGCCATCTGGGGTCCCGTCGCGGTTCAGAGGCAGTGGATCTGGGACACAGTTC
ACTCTCACCATCAGCGACATTCAGCGTGAGGATGCTGCCACCTACTATTGTCTAG
GCTGGCATGCTAGTACTGATGATGGTTGGGCATTCGGAGCTGGCACCAATGTGGG
AATCGAA
129H1
Amino acid sequence (SEQ ID NO: 359):
QSVKESGGGLFKPTDTLTLTCTVSGFSLSSNAITWVRQAPGKGLEWIGIIWSGGDTDY
ATWAKGRFTISKTSTTVDLEITSPTTEDTATYFCVKGATYSALWGPGTLVTVSS
Nucleic acid sequence (SEQ ID NO: 360):
CAGTCAGTGAAGGAGTCCGGGGGAGGCCTCTTCAAGCCAACGGATACCCTGACA
CTCACCTGCACCGTCTCTGGATTCTCCCTCAGTAGCAATGCAATAACTTGGGTCC
GCCAGGCTCCAGGGAAGGGGCTGGAATGGATCGGAATCATTTGGAGTGGTGGTG
ACACCGACTACGCGACCTGGGCGAAAGGCCGCTTCACCATCTCCAAAACCTCGA
CCACGGTGGATCTGGAAATCACCAGTCCGACAACCGAGGACACGGCCACCTATT
TCTGTGTCAAAGGGGCTACTTATAGTGCCTTGTGGGGCCCAGGCACCCTGGTCAC
CGTCTCCTCA
129L1
Amino acid sequence (SEQ ID NO: 361):
ALVMTQTPSSVEADVGGTVTIKCQASQSISSNYYAWYQQKPGQPPKWYKASTLAS
GVSSRFRGSGSGTEYTLTISDLECADAATYYCQGFDYGNSNVGAFGGGTEVVVK
Nucleic acid sequence (SEQ ID NO: 362):
GCCCTTGTGATGACCCAGACTCCATCCTCCGTGGAGGCAGATGTGGGAGGCACA
GTCACCATCAAGTGCCAGGCCAGTCAGAGTATTAGTAGTAACTACTATGCCTGGT
ATCAGCAGAAACCAGGGCAGCCTCCCAAGCTCCTGATCTACAAGGCATCCACTCT
GGCATCTGGGGTCTCATCGCGGTTCAGAGGCAGTGGATCTGGGACAGAGTATAC
TCTCACCATCAGCGACCTGGAGTGTGCCGATGCTGCCACTTACTACTGTCAAGGC
TTTGATTATGGTAATAGTAATGTTGGTGCTTTCGGCGGAGGGACCGAGGTGGTGG
TCAAA
134H1
Amino acid sequence (SEQ ID NO: 363):
QSLEESGGRLVTPGTPLTLTCTVSGIDLSSNAMSWVRQAPGKGLEWIGYIWSGGNTD
YASWAKGRFTISKTSTTVDLKITSPTTEDTATYFCARGGSYFPFWGPGTLVTVSS
Nucleic acid sequence (SEQ ID NO: 364):
CAGTCGCTGGAGGAGTCCGGGGGTCGCCTGGTCACGCCTGGGACACCCCTGACA
CTCACCTGCACAGTCTCTGGAATCGACCTCAGTAGCAATGCAATGAGCTGGGTCC
GCCAGGCTCCAGGGAAGGGGCTGGAATGGATCGGATACATTTGGAGTGGTGGTA
ATACAGACTACGCGAGCTGGGCGAAAGGCCGATTCACCATCTCCAAAACCTCGA
CCACGGTGGATCTGAAAATCACCAGTCCAACAACCGAGGACACGGCCACCTATT
TCTGTGCCAGGGGGGGGTCATACTTTCCCTTCTGGGGCCCAGGCACCCTGGTCAC
CGTCTCCTCA
134L2
Amino acid sequence (SEQ ID NO: 365):
DPVMTQTPSSTSAAVGGTVTINCQSSQSVYIDRLAWYQQKPGQPPKLLIYQASKLPS
GVPSRFSGSGSGKQSTLTISGVQCDDAATYYCAGFYDSGSGTYTLAFGGGTEVVVK
Nucleic acid sequence (SEQ ID NO: 366):
GACCCTGTGATGACCCAGACTCCATCTTCCACGTCTGCGGCTGTGGGAGGCACAG
TCACCATCAACTGCCAGTCCAGTCAGAGTGTTTATATCGACCGCTTAGCCTGGTA
TCAGCAGAAACCAGGGCAGCCTCCCAAGCTCCTGATCTACCAGGCATCCAAACT
GCCATCTGGGGTCCCATCGCGGTTCAGCGGCAGTGGATCTGGGAAACAGTCCACT
CTCACCATCAGTGGCGTGCAGTGTGACGATGCTGCCACTTACTACTGTGCAGGGT
TTTATGATAGTGGTAGTGGCACTTATACATTAGCTTTCGGCGGAGGGACCGAGGT
GGTGGTCAAA
151H1
Amino acid sequence (SEQ ID NO: 367):
QSVEESGGRLVTPGTPLTLTCTVSGFSLSSNAISWVRQAPGKGLEWIGVIDAGGSTYF
ASWAKGLFTISKTSSTTVDLQMTSLTTEDTATYFCARGWSRHDFWGPGTLVTVSS
Nucleic acid sequence (SEQ ID NO: 368):
CAGTCGGTGGAGGAGTCCGGGGGTCGCCTGGTCACGCCTGGGACACCCCTGACA
CTCACCTGCACCGTCTCTGGATTCTCCCTCAGTAGCAATGCAATAAGCTGGGTCC
GCCAGGCTCCAGGGAAGGGGCTGGAATGGATCGGAGTCATTGATGCTGGTGGGT
CCACATACTTCGCGAGCTGGGCGAAAGGCCTATTCACCATCTCCAAAACCTCGTC
GACCACGGTGGATCTGCAAATGACCAGTCTGACAACCGAGGACACGGCCACCTA
TTTCTGTGCCAGAGGTTGGAGTAGACATGACTTCTGGGGCCCAGGCACCCTGGTC
ACCGTCTCCTCA
151L1
Amino acid sequence (SEQ ID NO: 369):
DVVMTQTPASVSEPVGGTVTIKCQASQSISNILAWYQQKPGQPPRLLIYSASTLASGV
SSRFKASGSGTEFTLTISDLECADAATYYCQGYDSTVGVGAFGGGTEVVVK
Nucleic acid sequence (SEQ ID NO: 370):
GATGTTGTGATGACCCAGACTCCAGCCTCCGTGTCTGAACCTGTGGGAGGCACAG
TCACCATCAAGTGCCAGGCCAGTCAGAGCATTAGCAATATATTAGCCTGGTATCA
GCAGAAACCAGGGCAGCCTCCCAGGCTCCTGATCTATTCTGCATCCACTCTGGCA
TCTGGGGTCTCATCGCGGTTCAAGGCCAGTGGATCTGGGACAGAGTTCACTCTCA
CCATCAGCGACCTGGAGTGTGCCGATGCTGCCACTTACTACTGTCAAGGGTATGA
TAGTACTGTTGGTGTGGGTGCTTTCGGCGGAGGGACCGAGGTGGTGGTCAAA
166H2
Amino acid sequence (SEQ ID NO: 371):
QSLEESGGRLVTPGTPLTLTCTASGFDLSRYHMNWVRQAPGKGLEWIGIIYVSDDSY
YASWAKGRFTISKTSTAVDLKITSPTTEDTATYFCARVGSVWSSKLWGPGTLVTVSS
Nucleic acid sequence (SEQ ID NO: 372):
CAGTCGCTGGAGGAGTCCGGGGGTCGCCTGGTCACGCCTGGGACACCCCTGACA
CTCACCTGCACAGCCTCTGGATTCGACTTAAGTAGGTACCACATGAACTGGGTCC
GCCAGGCTCCAGGGAAGGGGCTGGAATGGATCGGAATCATTTATGTTAGTGATG
ACTCATACTACGCGAGCTGGGCGAAAGGCCGATTCACCATCTCCAAAACCTCGA
CCGCGGTGGATCTGAAAATCACCAGTCCGACAACCGAGGACACGGCCACCTATT
TCTGTGCCAGAGTTGGTAGTGTTTGGAGCAGTAAGTTGTGGGGCCCAGGCACCCT
GGTCACCGTCTCCTCA
166L1
Amino acid sequence (SEQ ID NO: 373):
DVVMTQTPASVSEPVGGTVTIKCQASENIYNNLAWYQQKPGQPPKWYRASTLESG
VPSRFKGSGSGTEFTLTISDLECADAATYYCQSTYFGGSYVFAFGGGTEVVVK
Nucleic acid sequence (SEQ ID NO: 374):
GATGTTGTGATGACCCAGACTCCAGCCTCCGTGTCTGAACCTGTGGGAGGCACAG
TCACCATCAAGTGCCAGGCCAGTGAGAACATTTACAACAATTTAGCCTGGTATCA
GCAGAAACCAGGGCAGCCTCCCAAGCTCCTGATCTACAGGGCATCCACTCTGGA
ATCTGGGGTCCCATCGCGGTTCAAAGGCAGTGGATCTGGGACAGAGTTCACTCTC
ACCATCAGCGACCTGGAGTGCGCCGATGCTGCCACTTACTACTGTCAATCTACTT
ATTTTGGTGGGAGTTATGTTTTTGCTTTCGGCGGAGGGACCGAGGTGGTGGTCAA
A
167H2
Amino acid sequence (SEQ ID NO: 375):
QSLEESGGRLVTPGTPLTLTCTVSGFSLSSNAISWVRQAPGKGLEWIGTIYATDSTSY
ASWAKGRFTISKTSTTVDLKMTSLTAADTATYFCALGASYSALWGPGTLVTVSS
Nucleic acid sequence (SEQ ID NO: 376):
CAGTCGCTGGAGGAGTCCGGGGGTCGCCTGGTCACGCCTGGGACACCCCTGACA
CTCACCTGCACAGTCTCTGGATTCTCCCTCAGTAGCAATGCAATAAGTTGGGTCC
GCCAGGCTCCAGGGAAGGGGCTGGAATGGATCGGAACCATTTATGCTACTGATA
GCACGTCCTACGCGAGCTGGGCAAAAGGCCGATTCACCATCTCCAAAACCTCGA
CCACGGTGGATCTGAAAATGACCAGTCTGACAGCCGCGGACACGGCCACCTATT
TCTGTGCCTTAGGTGCTAGTTATTCTGCTTTGTGGGGCCCAGGCACCCTGGTCACC
GTCTCCTCA
167L2
Amino acid sequence (SEQ ID NO: 377):
AIVMTQTPSSKSVAVGDTVTINCQASESVASNDRLAWYQQKPGQRPKLLIYQASTLA
SGVPSRFKGSGSGTEFTLTISNVVCDDAATYYCAGYKSSSTDGNAFGGGTEVVVK
Nucleic acid sequence (SEQ ID NO: 378):
ATCGTGATGACCCAGACTCCATCTTCCAAGTCTGTCGCTGTGGGAGACACAGTCA
CCATCAATTGCCAGGCCAGTGAGAGTGTTGCTAGTAACGACCGCTTAGCCTGGTA
TCAGCAGAAACCAGGGCAGCGTCCCAAACTCCTAATCTACCAGGCATCCACTCTG
GCATCTGGGGTCCCATCGCGGTTCAAAGGCAGTGGATCTGGGACAGAGTTCACTC
TCACCATCAGCAATGTGGTGTGTGACGATGCTGCCACTTACTACTGTGCAGGATA
TAAAAGTAGTAGTACTGATGGTAATGCTTTCGGCGGAGGGACCGAGGTGGTGGT
CAAA
193H1
Amino acid sequence (SEQ ID NO: 379):
QSVEESGGGLVTPGGTLTLTCTASGFSLSSYDMSWVRRAPGKGLEWIGVIATGGRRD
YASWAKGRFTVSKTSTTVDLKMTSLTAADTATYFCARYSDSDGYALWGPGTLVTV
SS
Nucleic acid sequence (SEQ ID NO: 380):
CAGTCGGTGGAGGAGTCCGGAGGAGGCCTGGTAACGCCTGGAGGAACCCTGACA
CTCACCTGCACAGCCTCTGGATTCTCCCTCAGCAGCTACGACATGAGCTGGGTCC
GCCGGGCTCCAGGGAAGGGGCTGGAATGGATCGGAGTCATTGCTACTGGTGGTA
GAAGGGACTACGCGAGCTGGGCAAAAGGCTGATTCACCGTCTCCAAAACCTCGA
CCACGGTGGATCTGAAAATGACCAGTCTGACAGCCGCGGACACGGCCACCTATT
TCTGTGCCAGATATAGTGATAGTGATGGTTATGCCTTGTGGGGCCCAGGCACCCT
GGTCACCGTCTCCTCA
193L2
Amino acid sequence (SEQ ID NO: 381):
AEVVMTQAPASVEAAVGGTVTIKCQASESIGSWLAWYQQKPGQPPKWYSASTLAF
GVPSRFSGSGSGTQFTLTISDLECADAATYYCQSNYYSTSGHAFGGGTEVVVK
Nucleic acid sequence (SEQ ID NO: 382):
GCCGAAGTAGTGATGACCCAGGCTCCAGCCTCCGTGGAGGCAGCTGTGGGAGGC
ACAGTCACCATCAAGTGCCAGGCCAGTGAGAGCATTGGCAGTTGGTTAGCCTGG
TATCAGCAGAAACCAGGGCAGCCTCCCAAGCTCCTGATCTATTCTGCGTCCACTC
TGGCATTTGGGGTCCCGTCGCGGTTCAGCGGCAGTGGATCTGGGACACAGTTCAC
TCTCACCATCAGCGACCTGGAGTGTGCCGATGCGGCCACTTACTACTGTCAAAGT
AATTATTATAGTACTAGTGGGCATGCTTTCGGCGGAGGGACCGAGGTGGTGGTCA
AA
217H2
Amino acid sequence (SEQ ID NO: 383):
QSVEESGGRLVTPGTPLTLTCKASGFSLSNYWMNWVRQAPGKGLEWIGTINYGGST
YYASWAKGRFTISKTSTTVDLKITSPTTEDTATYFCARDNGAYTFDSWGPGTLVTVS
S
Nucleic acid sequence (SEQ ID NO: 384):
CAGTCGGTGGAGGAGTCCGGGGGTCGCCTGGTCACGCCTGGGACACCCCTGACA
CTCACCTGCAAAGCCTCTGGATTCTCCCTCAGTAACTACTGGATGAACTGGGTCC
GCCAGGCTCCAGGGAAGGGGCTGGAATGGATCGGAACCATTAATTATGGTGGTA
GCACATACTACGCGAGCTGGGCGAAAGGCCGATTCACCATCTCCAAAACCTCGA
CCACGGTGGATCTGAAAATCACCAGTCCGACAACCGAGGACACGGCCACCTATT
TCTGTGCCAGAGATAATGGTGCTTATACTTTTGATTCCTGGGGCCCAGGCACCCT
GGTCACCGTCTCCTCA
217L1
Amino acid sequence (SEQ ID NO: 385):
IVMTQTPSSASEPVGGTVTIKCQASQSVYNNNYLSWYQQKPGQSPKQLIYAASTLAS
GVPSRFKGSGSGTQFTLTISDVQCDDAASYYCLGKSSCSYDDCRAFGGGTEVVVK
Nucleic acid sequence (SEQ ID NO: 386):
ATTGTGATGACCCAGACTCCATCCTCTGCGTCTGAACCTGTGGGAGGCACAGTCA
CCATCAAATGCCAGGCCAGTCAGAGTGTTTATAATAACAACTACTTATCCTGGTA
TCAGCAGAAACCAGGGCAGTCTCCCAAGCAACTGATCTATGCTGCATCCACTCTG
GCATCTGGGGTCCCATCGCGGTTCAAAGGCAGTGGATCTGGGACACAGTTCACTC
TCACCATCAGCGACGTGCAGTGTGACGATGCTGCCAGTTACTACTGTCTAGGCAA
ATCTAGTTGTAGTTATGATGATTGTAGGGCTTTCGGCGGAGGGACCGAGGTGGTG
GTCAAA
233H1
Amino acid sequence (SEQ ID NO: 387):
QEQLVESGGGLVQPEGSLTLTCTASGFSFSYYSWACWVRQAPGKGLEWIACIDGGG
SRATYYASWAKGRFTISTTSSTTVTLQMTSLTAADTATYFCSRSDYNGYISYFDLWG
PGTLVTVSS
Nucleic acid sequence (SEQ ID NO: 388):
CAGGAGCAGCTGGTGGAGTCCGGGGGAGGCCTGGTCCAGCCTGAGGGATCCCTG
ACACTCACCTGCACAGCCTCTGGATTCTCCTTTAGTTACTATTCTTGGGCGTGCTG
GGTCCGCCAGGCTCCAGGGAAGGGACTGGAGTGGATCGCATGCATTGATGGTGG
TGGTAGTCGCGCCACTTACTACGCGAGCTGGGCGAAAGGCCGATTCACCATCTCC
ACAACCTCGTCGACCACGGTGACTCTGCAAATGACCAGTCTGACAGCCGCGGAC
ACGGCCACTTATTTCTGTTCGAGATCCGACTATAATGGTTATATCTCCTACTTTGA
CTTGTGGGGCCCCGGCACCCTGGTCACCGTCTCCTCA
233L1
Amino acid sequence (SEQ ID NO: 389):
AFELTQTPSSVEAAVGGTVTINCQASQSIRSDLAWYQQKPGQPPKWYKASTLASGV
PSRFRGSGSGTEYTLTISDLECADAATYYCQSYYHSSSTAFGGGTEVVVK
Nucleic acid sequence (SEQ ID NO: 390):
GCATTCGAGTTGACCCAGACTCCATCCTCCGTGGAGGCAGCTGTGGGAGGCACA
GTCACCATCAATTGCCAGGCCAGTCAGAGCATTCGTAGCGACTTAGCCTGGTATC
AGCAGAAACCAGGGCAGCCTCCCAAGCTCCTGATCTATAAGGCATCCACTCTGG
CATCTGGGGTCCCATCGCGGTTCAGAGGCAGTGGATCTGGGACAGAGTACACTCT
CACCATCAGCGACCTGGAGTGTGCCGATGCTGCCACTTACTACTGTCAAAGCTAT
TATCATAGTAGTAGTACTGCTTTCGGCGGAGGGACCGAGGTGGTGGTCAAA
169aH1
Amino acid sequence (SEQ ID NO: 391):
QSPEESGGRLVTPGTPLTLTCTVSGFSLSSNAISWVRQAPGKGLEWIGYIDANTNAYY
ASWAKGRVTISQTSTTVDLRITSPTSEDTATYFCARGVTYYPMWGPGTLVTVSS
Nucleic acid sequence (SEQ ID NO: 392):
CAGTCGCCGGAGGAGTCCGGGGGTCGCCTGGTCACGCCTGGGACACCCCTGACA
CTCACCTGCACCGTCTCTGGATTCTCCCTCAGTAGCAATGCAATAAGCTGGGTCC
GCCAGGCTCCAGGGAAGGGGCTGGAGTGGATCGGATATATAGATGCGAATACTA
ACGCATACTACGCGAGTTGGGCAAAAGGCCGAGTCACCATCTCCCAAACCTCGA
CCACGGTGGATCTGAGGATCACCAGTCCGACAAGCGAAGACACGGCCACCTATT
TCTGTGCCAGAGGTGTTACTTATTATCCAATGTGGGGCCCAGGCACCCTGGTCAC
CGTCTCCTCA
169aL1
Amino acid sequence (SEQ ID NO: 393):
DVVMTQTPASVSEPVGGTVTIKCQASQSIASRYCSWYQQKPGQPPKLLIYKASTLAS
GVSSRFKGSGSETEFTLTISDLECADAATYYCQGGYYGDSYVGAFGGGTEVVVK
Nucleic acid sequence (SEQ ID NO: 394):
GATGTTGTGATGACCCAGACTCCAGCCTCCGTGTCTGAACCTGTGGGAGGCACAG
TCACCATCAAGTGCCAGGCCAGTCAGAGTATTGCTAGTAGGTACTGCTCCTGGTA
TCAGCAGAAACCAGGGCAGCCTCCCAAGCTCCTGATCTACAAGGCATCCACTCTA
GCATCTGGGGTCTCATCGCGGTTCAAAGGCAGTGGATCTGAGACAGAGTTCACTC
TCACCATCAGCGACCTGGAGTGTGCCGATGCTGCCACTTACTACTGTCAAGGCGG
TTATTATGGTGATAGTTATGTTGGCGCTTTCGGCGGAGGGACCGAGGTGGTGGTC
AAA
176aH1
Amino acid sequence (SEQ ID NO: 395):
QSVEESGGRLVKPDETLTLTCTVSGFSLSSNAISWVRQAPGKGLEWIGAIYSDDNTYY
ANWAKGRFTISKTSTTVDLKMTSLTTEDTATYFCGRGASRFDFWGPGTLVTVSS
Nucleic acid sequence (SEQ ID NO: 396):
CAGTCGGTGGAGGAGTCCGGGGGTCGCCTGGTCAAGCCTGACGAAACCCTGACA
CTCACCTGCACCGTCTCTGGATTCTCCCTCAGTAGCAATGCAATAAGTTGGGTCC
GCCAGGCTCCAGGGAAGGGGCTGGAATGGATCGGAGCCATTTATAGTGATGATA
ACACATACTACGCGAACTGGGCGAAAGGCCGATTCACCATCTCCAAAACCTCGA
CCACGGTGGATCTGAAAATGACCAGTCTGACAACCGAGGACACGGCCACCTATT
TCTGTGGCAGAGGTGCTTCTAGGTTTGACTTCTGGGGCCCAGGCACCCTGGTCAC
CGTCTCCTCA
176aL1
Amino acid sequence (SEQ ID NO: 397):
DVVMTQTPASVSAAVGGTVTIKCQASQSINGNYLAWYQQKPGQPPKWYKASTLTS
GVPSRFKGSGSGTQFTLTISDLECADGATYYCQYTDYGSTYVGAFGGGTEVVVK
Nucleic acid sequence (SEQ ID NO: 398):
GATGTTGTGATGACCCAGACTCCAGCCTCCGTGTCTGCAGCTGTGGGAGGCACAG
TCACCATCAAGTGCCAGGCCAGTCAGAGTATTAATGGTAACTACTTAGCCTGGTA
TCAGCAGAAACCAGGGCAGCCTCCCAAGCTCCTAATCTATAAGGCATCCACTCTG
ACATCTGGGGTCCCATCGCGGTTCAAAGGCAGTGGATCTGGGACACAGTTCACTC
TCACCATCAGCGACCTGGAGTGTGCCGATGGTGCCACTTACTACTGTCAATATAC
TGATTATGGTAGTACTTATGTTGGTGCTTTCGGCGGAGGGACCGAGGTGGTGGTC
AAA
181aH1
Amino acid sequence (SEQ ID NO: 399):
QSLEESGGRLVTPGTPLTLTCTVSGFSLSSNAISWVRQAPGKGLEWIGYIDANTNAYY
ASWAKGRVTISQTSTTVDLRITSPTSEDTATYFCARGVTYYPMWGPGTLVTVSS
Nucleic acid sequence (SEQ ID NO: 400):
CAATCGCTGGAGGAGTCCGGGGGTCGCCTGGTCACGCCTGGGACACCCCTGACG
CTCACCTGCACCGTCTCTGGATTCTCCCTCAGTAGCAATGCAATAAGCTGGGTCC
GCCAGGCTCCAGGGAAGGGGCTGGAGTGGATCGGATATATAGATGCGAATACTA
ACGCATACTACGCGAGTTGGGCAAAAGGCCGAGTCACCATCTCCCAAACCTCGA
CCACGGTGGATCTGAGGATCACCAGTCCGACAAGCGAAGACACGGCCACCTATT
TCTGTGCCAGAGGTGTTACTTATTATCCAATGTGGGGCCCAGGCACCCTGGTCAC
CGTCTCCTCA
181aL1
Amino acid sequence (SEQ ID NO: 401):
DVVMTQTPASVSEPVGGTVTIKCQASQSIGSRYWSWYQQQPGQPPKWYKASTLAS
GVSSRFKGSGSETEFTLTISDLECADAATYYCQGGYYGDSYVGAFGGGTEVVVK
Nucleic acid sequence (SEQ ID NO: 402):
GATGTTGTGATGACCCAGACTCCAGCCTCCGTGTCTGAACCTGTGGGAGGCACAG
TCACCATCAAGTGCCAGGCCAGTCAGAGTATTGGTAGTAGGTACTGGTCCTGGTA
TCAGCAGCAACCAGGGCAGCCTCCCAAGCTCCTGATCTACAAGGCATCCACTCTG
GCATCTGGGGTCTCATCGCGGTTCAAAGGCAGTGGATCTGAGACAGAGTTCACTC
TCACCATCAGCGACCTGGAGTGTGCCGATGCTGCCACTTACTACTGTCAAGGCGG
TTATTATGGTGATAGTTATGTTGGCGCTTTCGGCGGAGGGACCGAGGTGGTGGTC
AAA
183aH2
Amino acid sequence (SEQ ID NO: 403):
QSVEESGGRLVTPGTPLTITCTVSGMDLSSNAMTWVRQAPGKGLEWIGIIYASDSTY
YASWAKGRFTISKTSSTTVDLKITSPTTEDTATYFCARGATYIPLWGPGTLVTVSS
Nucleic acid sequence (SEQ ID NO: 404):
CAGTCGGTGGAGGAGTCCGGGGGTCGCCTGGTCACGCCTGGGACACCCCTGACA
ATCACCTGCACAGTCTCTGGAATGGACCTCAGTAGCAATGCAATGACCTGGGTCC
GCCAGGCTCCAGGGAAGGGGCTGGAATGGATTGGAATCATTTATGCTAGTGATA
GCACATACTACGCGAGCTGGGCGAAAGGCCGATTCACCATCTCCAAAACCTCGT
CGACCACGGTGGATCTAAAAATCACCAGTCCGACAACCGAGGACACGGCCACCT
ATTTCTGTGCCAGAGGTGCTACTTACATTCCCTTGTGGGGCCCAGGCACCCTGGT
CACCGTCTCCTCA
183aL1
Amino acid sequence (SEQ ID NO: 405):
DVVMTQTPASVSEPVGGTVTINCQASQSISSSYLAWYQQKPGQPPKLLIYKASTLASG
VSSRFKGSGSGTEFTLTISDLECADAATYYCQCTDYGSSYVGTFGGGTEVVVK
Nucleic acid sequence (SEQ ID NO: 406):
GATGTTGTGATGACCCAGACTCCAGCCTCCGTGTCTGAACCTGTGGGAGGCACAG
TCACCATCAATTGCCAGGCCAGTCAGAGTATTAGTAGTAGCTACTTAGCCTGGTA
TCAGCAGAAACCAGGGCAGCCTCCCAAGCTCCTGATCTACAAGGCATCCACTCTG
GCATCTGGGGTCTCATCGCGGTTTAAAGGCAGTGGATCTGGGACAGAGTTCACTC
TCACCATCAGCGACCTGGAGTGTGCCGATGCTGCCACTTACTACTGTCAATGTAC
TGATTATGGTAGTAGTTATGTTGGTACTTTCGGCGGAGGGACCGAGGTGGTGGTC
AAA
184aH1
Amino acid sequence (SEQ ID NO: 407):
QSVEESGGRLVKPDETLTLTCTVSGIDLSSNTMSWVRQAPGKGLEWIGLIGPVSNTY
YANWAKGRVTISKTSTTVDLKITSPTTEDTATYFCARGWFQYSFWGPGTLVTVSS
Nucleic acid sequence (SEQ ID NO: 408):
CAGTCGGTGGAGGAGTCCGGGGGTCGCCTGGTCAAGCCTGACGAAACCCTGACA
CTCACCTGCACAGTGTCTGGAATCGACCTCAGTAGCAATACAATGAGCTGGGTCC
GCCAGGCTCCAGGGAAGGGGCTGGAATGGATCGGACTGATTGGTCCTGTCAGTA
ACACATACTACGCGAACTGGGCGAAAGGCCGGGTCACCATCTCCAAAACCTCGA
CCACGGTGGATCTGAAAATCACCAGTCCGACAACCGAGGACACGGCCACCTATT
TCTGTGCCAGAGGCTGGTTCCAATATAGCTTCTGGGGCCCAGGCACCCTGGTCAC
CGTCTCCTCA
184aL1
Amino acid sequence (SEQ ID NO: 409):
AEVLMTQTPSSVEAPVGGTVTINCQASQSIDSYLSWYQQKPGQPPKWYKASTLASG
VSSRFKGSGSGTEFTLTISDLECADAATYYCQGGYYSSSNNYITFGGGTEVVVK
Nucleic acid sequence (SEQ ID NO: 410):
GCCGAAGTACTGATGACCCAGACTCCATCCTCCGTGGAGGCACCTGTGGGAGGC
ACAGTCACCATCAACTGCCAGGCCAGTCAGAGCATTGATAGCTACTTATCCTGGT
ATCAGCAGAAACCAGGGCAGCCTCCCAAGCTCCTGATCTACAAGGCATCCACTCT
GGCATCTGGGGTCTCATCGCGGTTCAAAGGCAGTGGATCTGGGACAGAGTTCACT
CTCACCATCAGCGACCTGGAGTGTGCCGATGCTGCCACTTACTACTGTCAAGGCG
GTTATTATAGTAGTAGTAATAATTATATTACTTTCGGCGGAGGGACCGAGGTGGT
GGTCAAA
5112-z
Amino acid sequence (SEQ ID NO: 411):
EVQLVESGGGLVQPGGSLRLSCAASGFSLSSNAISWVRQAPGKGLEWVGIIYASGDT
YYASWAKGRFTISRDNSKNTLYLQMNSLRAEDTAVYYCARGYTTLYFWGQGTLVT
VSS
Nucleic acid sequence (SEQ ID NO: 412):
GAGGTGCAGCTGGTGGAGTCCGGAGGAGGACTGGTGCAGCCAGGAGGCAGCCTG
AGGCTGTCCTGTGCAGCCTCCGGCTTCTCTCTGAGCTCCAACGCCATCTCTTGGGT
GAGGCAGGCACCTGGCAAGGGACTGGAGTGGGTGGGCATCATCTACGCCTCCGG
CGACACCTACTATGCCTCTTGGGCCAAGGGCCGGTTCACCATCTCTAGAGATAAC
AGCAAGAATACACTGTATCTGCAGATGAATTCCCTGAGGGCCGAGGACACAGCC
GTGTACTATTGCGCCCGCGGCTACACCACACTGTATTTTTGGGGCCAGGGCACCC
TGGTGACAGTGTCTAGC
5L2-z
Amino acid sequence (SEQ ID NO: 413):
DIQMTQSPSSVSASVGDRVTITCQASESISTRLAWYQQKPGKAPKLLIYSASTLPSGVP
SRFSGSGSGTDFTLTISSLQPEDFATYYCQGGYSSGAGTAFGGGTKVEIK
Nucleic acid sequence (SEQ ID NO: 414):
GACATCCAGATGACACAGAGCCCAAGCTCCGTGAGCGCCTCCGTGGGCGATAGG
GTGACCATCACATGTCAGGCCTCTGAGAGCATCTCCACCAGGCTGGCATGGTACC
AGCAGAAGCCAGGCAAGGCCCCTAAGCTGCTGATCTATTCTGCCAGCACCCTGCC
ATCCGGAGTGCCATCTAGGTTCTCCGGCTCTGGCAGCGGCACAGACTTTACCCTG
ACAATCTCTAGCCTGCAGCCCGAGGATTTCGCCACCTACTATTGCCAGGGAGGAT
ACTCCTCTGGAGCAGGAACCGCCTTTGGCGGAGGCACAAAGGTGGAGATCAAG
6H2-z
Amino acid sequence (SEQ ID NO: 415):
EVQLVESGGGLVQPGGSLRLSCAASGFDFSRYHMSWVRQAPGKGLEWVGIIYVSDN
TYYATWAKGRFTISRDNSKNTLYLQMNSLRAEDTAVYYCVRVGSFWSSKLWGQGT
LVTVSS
Nucleic acid sequence (SEQ ID NO: 416):
GAGGTGCAGCTGGTGGAGAGCGGAGGAGGACTGGTGCAGCCAGGAGGCTCCCTG
CGGCTGTCTTGCGCCGCCAGCGGCTTCGATTTTTCCAGGTACCACATGTCCTGGG
TGCGCCAGGCACCTGGCAAGGGACTGGAGTGGGTGGGCATCATCTACGTGAGCG
ACAACACCTACTATGCCACATGGGCCAAGGGCCGGTTCACCATCTCCAGAGATA
ACTCTAAGAATACACTGTACCTGCAGATGAATAGCCTGAGGGCAGAGGACACCG
CCGTGTACTATTGCGTGCGGGTGGGCTCCTTTTGGAGCTCCAAGCTGTGGGGACA
GGGCACCCTGGTGACAGTGTCTAGC
6L2-z
Amino acid sequence (SEQ ID NO: 417):
DIQMTQSPSTLSASVGDRVTITCQASENIYSSLAWYQQKPGKAPKLLIYEASNLESGV
PSRFSGSGSGTEFTLTISSLQPDDFATYYCQSTYFGNSYVFAFGGGTKVEIK
Nucleic acid sequence (SEQ ID NO: 418):
GACATCCAGATGACCCAGTCCCCATCTACACTGAGCGCCTCCGTGGGCGATAGG
GTGACCATCACATGTCAGGCCAGCGAGAACATCTACAGCTCCCTGGCCTGGTATC
AGCAGAAGCCCGGCAAGGCCCCTAAGCTGCTGATCTACGAGGCCTCTAATCTGG
AGAGCGGAGTGCCATCCCGGTTCTCTGGAAGCGGATCCGGAACCGAGTTTACCCT
GACAATCTCTAGCCTGCAGCCCGACGATTTCGCCACCTACTATTGCCAGTCTACA
TACTTTGGCAACAGCTACGTGTTCGCCTTTGGCGGCGGCACAAAGGTGGAGATCA
AG

CDRs are known to be responsible for antigen binding, however, it has been found that not all of the 6 CDRs are indispensable or unchangeable. In other words, it is possible to replace or change or modify one or more CDRs in anti-CD40 antibody clone 1, 3, 5, 6, 12, 16, 17, 24, 26, 27, 31, 45, 58, 70, 78, 86, 91, 93, 94, 102, 103, 105, 108, 109, 110, 114, 120, 121, 129, 134, 151, 166, 167, 193, 217, 233, 169a, 176a, 181a, 183, 184, 5-z, or 6-z, yet substantially retain the specific binding affinity to CD40.

In certain embodiments, the anti-CD40 antibodies and the antigen-binding fragments provided herein comprise a heavy chain CDR3 sequence of one of anti-CD40 antibody clone 1, 3, 5, 6, 12, 16, 17, 24, 26, 27, 31, 45, 58, 70, 78, 86, 91, 93, 94, 102, 103, 105, 108, 109, 110, 114, 120, 121, 129, 134, 151, 166, 167, 193, 217, 233, 169a, 176a, 181a, 183, 184, 5-z, or 6-z. In certain embodiments, the anti-CD40 antibodies and the antigen-binding fragments provided herein comprise a heavy chain CDR3 sequence selected from the group consisting of SEQ ID NOs: 5, 11, 17, 23, 29, 35, 41, 47, 53, 59, 65, 71, 77, 83, 89, 95, 101, 107, 113, 119, 125, 131, 137, 143, 149, 155, 161, 167, 173, 179, 185, 191, 197, 203, 209, 215, 221, 227, 233, 239, and 245. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In certain embodiments, the antibodies and antigen-binding fragments thereof provided herein comprise suitable framework region (FR) sequences, as long as the antibodies and antigen-binding fragments thereof can specifically bind to CD40. The CDR sequences provided in Table 1 are obtained from rabbit antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et at. (1992) Proc. Natl. Acad. Sci. USA, 89:4285; Presta et al. (1993) J. Immunol., 151:2623).

In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody. In some embodiments, the humanized antibody or antigen-binding fragment comprise human FR1-4.

In certain embodiments, the humanized antibodies and antigen-binding fragment thereof provided herein comprise one or more FR sequences of antibody clone 5-z or 6-z.

The two exemplary humanized anti-CD40 antibodies clone 5-z and 6-z both retained the specific binding affinity to CD40-expressing cell, and are at least comparable to, or even better than, the parent rabbit antibodies in that aspect. The two exemplary humanized antibodies both retained their functional interaction with CD40-expressing cell, in that both can induce human B cell activation and induce human dendritic cell maturation and activation.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure. In certain embodiments, the humanized antibody or antigen-binding fragment provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains.

In certain embodiments, the antibodies and antigen-binding fragments thereof provided herein comprise a heavy chain variable domain sequence selected from the group consisting of SEQ ID NOs: 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, and 415. In certain embodiments, the antibodies and antigen-binding fragments thereof provided herein comprise a light chain variable domain sequence selected from the group consisting of SEQ ID NOs: 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 401, 405, 409, 413, and 417.

In some embodiments, the anti-CD40 antibodies and the antigen-binding fragments provided herein comprise all or a portion of the heavy chain variable domain and/or all or a portion of the light chain variable domain. In one embodiment, the anti-CD40 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-CD40 antibodies and the fragments thereof provided herein further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

The antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

### IV. Antibody Variants

The antibodies and antigen-binding fragments thereof provided herein also encompass various variants thereof. In certain embodiments, the antibodies and antigen-binding fragments thereof encompasses various types of variants of an exemplary antibody provided herein, i.e., antibody clone 1, 3, 5, 6, 12, 16, 17, 24, 26, 27, 31, 45, 58, 70, 78, 86, 91, 93, 94, 102, 103, 105, 108, 109, 110, 114, 120, 121, 129, 134, 151, 166, 167, 193, 217, 233, 169a, 176a, 181a, 183, 184, 5-z, and 6-z.

In certain embodiments, the antibody variants comprise one or more modifications or substitutions in one or more CDR sequences as provided in Table 1, one or more variable region sequences (but not in any of the CDR sequences) provided herein, and/or the constant region (e.g. Fc region). Such variants retain specific binding affinity to CD40 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

Affinity Variant

Affinity variant may contain modifications or substitutions in one or more CDR sequences as provided in Table 1, one or more FR sequences, or the heavy or light chain variable region sequences provided herein. FR sequences can be readily identified by a skilled person in the art based on the CDR sequences in Table 1 and variable region sequences herein, as it is well-known in the art that a CDR region is flanked by two FR regions in the variable region. The affinity variants retain specific binding affinity to CD40 of the parent antibody, or even have improved CD40 specific binding affinity over the parent antibody. In certain embodiments, at least one (or all) of the substitution(s) in the CDR sequences, FR sequences, or variable region sequences comprises a conservative substitution.

A skilled artisan will understand that in the CDR sequences provided in Table 1 and variable region sequences provided herein, one or more amino acid residues may be substituted yet the resulting antibody or antigen-binding fragment still retain the binding affinity to CD40, or even have an improved binding affinity. Various methods known in the art can be used to achieve this purpose. For example, a library of antibody variants (such as Fab or scFv variants) can be generated and expressed with phage display technology, and then screened for the binding affinity to human CD40. For another example, computer software can be used to virtually simulate the binding of the antibodies to human CD40, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in the CDR sequences and/or FR sequences in total.

In certain embodiments, the anti-CD40 antibodies and antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to CD40 at a level similar to or even higher than its parent antibody.

In certain embodiments, the anti-CD40 antibodies and antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) provided herein, and in the meantime retain the binding affinity to CD40 at a level similar to or even higher than its parent antibody. In some embodiments, a total of 1 to 10 amino acids have been substituted, inserted, or deleted in a variable region sequence of SEQ ID NOs: 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415 and 417. In some embodiments, the substitutions, insertions, or deletions occur in regions outside the CDRs (e.g., in the FRs).

Glycosylation Variant

The anti-CD40 antibodies and antigen-binding fragments provided herein also encompass a glycosylation variant, which can be obtained to either increase or decrease the extent of glycosylation of the antibody or antigen binding fragment.

The antibody or antigen binding fragment thereof may comprise one or more amino acid residues with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

Cysteine-Engineered Variant

The anti-CD40 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

Fc Variant

The anti-CD40 antibodies and antigen-binding fragments provided herein also encompass an Fc variant, which comprises one or more amino acid residue modifications or substitutions at its Fc region and/or hinge region.

In certain embodiments, the anti-CD40 antibodies or antigen-binding fragments comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

In certain embodiments, the anti-CD40 antibodies or antigen-binding fragments comprise one or more amino acid substitution(s) that alters the antibody-dependent cellular cytotoxicity (ADCC). Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O., et al., Mol Cancer Ther. 2008, 7(8): 2517-27; Shields R. L. et al, J. Biol. Chem, 2002, 277: 26733-26740; Shinkawa T. et al, J. Biol. Chem, 2003, 278: 3466-3473.

In certain embodiments, the anti-CD40 antibodies or antigen-binding fragments comprise one or more amino acid substitution(s) that alters Complement Dependent Cytotoxicity (CDC), for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fe region variants.

In certain embodiments, the anti-CD40 antibodies or antigen-binding fragments comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### V. Antigen-Binding Fragments

Provided herein are also anti-CD40 antigen-binding fragments. Various types of antigen-binding fragments are known in the art and can be developed based on the anti-CD40 antibodies provided herein, including for example, the exemplary antibodies whose CDR are shown in Table 1 and variable sequences are shown herein, and their different variants (such as affinity variants, glycosylation variants, Fc variants, cysteine-engineered variants and so on).

In certain embodiments, an anti-CD40 antigen-binding fragment provided herein is a camelized single domain antibody, a diabody, a single chain Fv fragment (scFv), an scFv dimer, a BsFv, a dsFv, a (dsFv)₂, a dsFv-dsFv′, an Fv fragment, a Fab, a Fab′, a F(ab′)₂, a bispecific antibody, a ds diabody, a nanobody, a domain antibody, a single domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phase display library as discussed above (e.g. for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck).

### VI. Conjugates

In some embodiments, the anti-CD40 antibodies and antigen-binding fragments thereof further comprise a conjugate moiety. The conjugate moiety can be linked to the antibodies and antigen-binding fragments thereof. A conjugate moiety is a non-proteinaceous moiety that can be attached to the antibody or antigen-binding fragment thereof. It is contemplated that a variety of conjugate moieties may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments by covalent binding, affinity binding, intercalation, coordinate binding, complexation, association, blending, or addition, among other methods.

In certain embodiments, the antibodies and antigen-binding fragments disclosed herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies may be linked to a conjugate moiety indirectly, or through another conjugate moiety. For example, the antibody or antigen-binding fragments may be conjugated to biotin, then indirectly conjugated to a second conjugate that is conjugated to avidin. The conjugate can be a clearance-modifying agent, a toxin (e.g., a chemotherapeutic agent), a detectable label (e.g., a radioactive isotope, a lanthanide, a luminescent label, a fluorescent label, or an enzyme-substrate label), or purification moiety.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection.

In certain embodiments, the conjugate moiety can be a clearance-modifying agent which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibodies and antigen-binding fragments thereof provided herein is used for a base for a conjugate.

### VII. Polynucleotides and Recombinant Methods

The present disclosure provides isolated polynucleotides that encode the anti-CD40 antibodies and antigen-binding fragments thereof. In certain embodiments, the isolated polynucleotides comprise one or more nucleotide sequences as shown in SEQ IN NO: 82, 84, 86, 88, 90, 92, 94, 96, 98, 100, 102, 104, 106, 108, 110, 112, 114, 116, 118, and/or 120, which encodes the variable region of the exemplary antibodies provided herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-CD40 antibodies and antigen-binding fragments thereof (e.g. including SEQ ID NOs: 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, and 418) can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors (e.g., expression vectors) containing the nucleic acid sequence provided herein encoding the antibodies or antigen-binding fragments, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenformis, Pseudomonas such as P. aeruginosa, and Streptomyces.

In addition to prokaryotes, eukaryotic microbes such as filamentous fungi or yeast are suitable cloning or expression hosts for anti-CD40 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is 293F cell.

Host cells are transformed with the above-described expression or cloning vectors for anti-CD40 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CD40 antibodies and antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### VIII. Pharmaceutical Composition

The present disclosure further provides pharmaceutical compositions comprising the anti-CD40 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CD40 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### IX. Methods of Use

The present disclosure also provides therapeutic methods comprising: administering a therapeutically effective amount of the antibody or antigen-binding fragment as provided herein to a subject in need thereof, thereby treating or preventing a CD40-related condition or a disorder. In some embodiment, the CD40-related condition or a disorder is cancer, autoimmune disease, inflammatory disease, or infectious disease.

Examples of cancer include but are not limited to, non-small cell lung cancer (squamous/nonsquamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

Autoimmune diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis. Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury. In some embodiments, the CD3 associated conditions are inflammatory diseases such as systemic lupus erythematosus (SLE), intestinal mucosal inflammation, wasting disease associated with colitis, multiple sclerosis, viral infections, rheumatoid arthritis, osteoarthritis, Cohn's disease, and inflammatory bowel disease, psoriasis, systemic scleroderma, autoimmune diabetes and the like.

Infectious disease include, but are not limited to, fungus infection, parasite/protozoan infection or chronic viral infection, for example, malaria, coccidioiodmycosis immitis, histoplasmosis, onychomycosis, aspergilosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV, cytomegalovirus, herpes simplex virus type I, herpes simplex virus type II, human papilloma virus, adenovirus, human immunodeficiency virus I, human immunodeficiency virus II, Kaposi West sarcoma associated herpes virus epidemics, thin ring virus (Torquetenovirus), human T lymphotrophic viruse I, human T lymphotrophic viruse II, varicella zoster, JC virus or BK virus.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the antibody or antigen-binding fragment as provided herein may be administered at a therapeutically effective dosage of about 0.01 mg/kg to about 100 mg/kg. In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments disclosed herein may be administered alone or in combination with one or more additional therapeutic means or agents. For example, the antibodies or antigen-binding fragments disclosed herein may be administered in combination with another therapeutic agent, for example, a chemotherapeutic agent or an anti-cancer drug.

In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of using the anti-CD40 antibodies or antigen-binding fragments thereof.

In some embodiments, the present disclosure provides methods of detecting presence or amount of CD40 in a sample, comprising contacting the sample with the antibody or antigen-binding fragment thereof, and determining the presence or the amount of CD40 in the sample.

In some embodiments, the present disclosure provides methods of diagnosing a CD40 related disease or condition in a subject, comprising: a) contacting a sample obtained from the subject with the antibody or antigen-binding fragment thereof provided herein; b) determining presence or amount of CD40 in the sample; and c) correlating the existence of the CD40 to the CD40 related disease or condition in the subject.

In some embodiments, the present disclosure provides kits comprising the antibody or antigen-binding fragment thereof provided herein, optionally conjugated with a detectable moiety. The kits may be useful in detection of CD40 or diagnosis of CD40 related disease.

In some embodiments, the present disclosure also provides use of the antibody or antigen-binding fragment thereof provided herein in the manufacture of a medicament for treating a CD40 related disease or condition in a subject, in the manufacture of a diagnostic reagent for diagnosing a CD40 related disease or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1: HEKBLUE Cell CD40 Activation

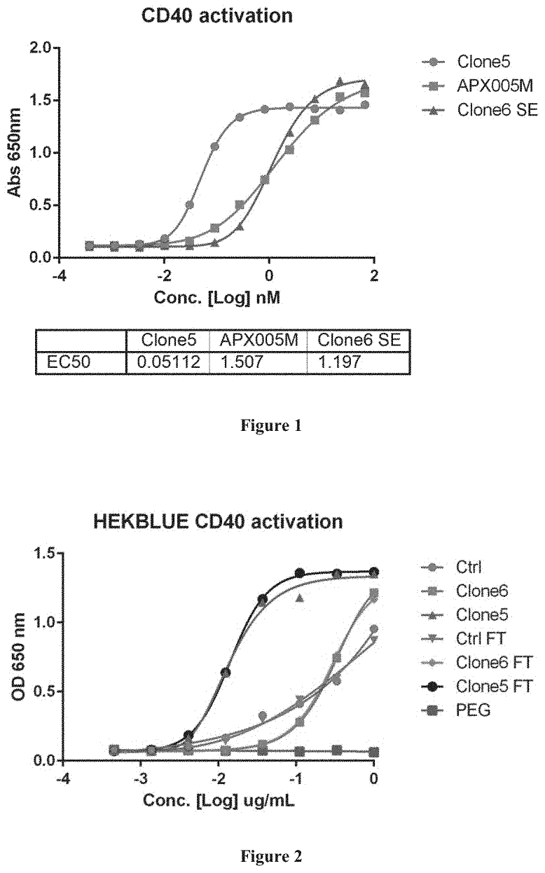

HEKBlue CD40L reporter cells (InvivoGen, San Diego, Calif., USA) were seeded at 5×10⁴/well in 96 well plate with anti-CD40 antibodies and incubated in 37° C. for 24 hours. Supernatant were analyzed for secreted embryonic alkaline phosphatase (SEAP) activity using Quanti-Blue assay (InvivoGen, San Diego, Calif., USA). Absorbance at 650 nm was plotted against antibody concentration to demonstrate CD40 activation. As shown in FIG. 1, antibody clone 5 has an EC50 of 0.34 nM, antibody clone 6 has an EC50 of 8.0 nM, while the control antibody (APX005M as disclosed in U.S. Pat. No. 8,778,345B2 to Zhang et al.) has an EC50 of 10 nM.

### Example 2: Stability Test Under Freeze-Thaw Condition

To test the stability of the antibodies under freeze-thaw condition, anti-CD40 antibody aliquots in PBS were froze in −80° C. for 72 hours and fully thawed at 4° C., and were then seeded at 5×10⁴/well in 96 well plate with HEKBlue CD40L reporter cells and incubated in 37° C. for 24 hours. Supernatant were analyzed for secreted embryonic alkaline phosphatase (SEAP) activity using Quanti-Blue assay. Absorbance at 650 nm was plotted against antibody concentration to demonstrate CD40 activation. As shown in FIG. 2, antibody clone 5 and 6 are stable under freeze-thaw condition. FT, Freeze-Thaw Aliquot

### Example 3: CD40 Binding on HEKBlue Cells

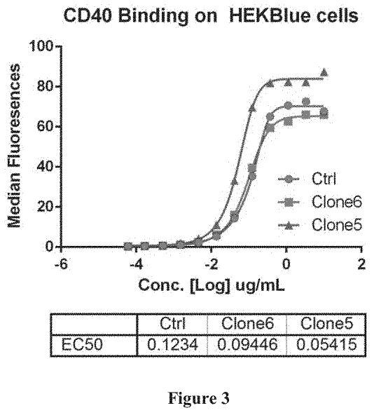

Flow cytometry were used to determine the binding of anti-CD40 antibody to CD40 protein displayed on cell surface. HEKBlue CD40L reporter cells were harvested and stained with anti-CD40 and fluorescent secondary antibodies. Antibodies binding are shown in median fluorescence intensity. As shown in FIG. 3, antibody clone 5 has an EC50 of 0.36 nM in binding to CD40, antibody clone 6 has an EC50 of 0.63 nM, while the control antibody has an EC50 of 0.82 nM.

### Example 4: Binding Affinity

Binding affinity to recombinant CD40 ECD-Fc fusion protein were determined using Bio-Layer Interferometry (BLI) on Octet system. Briefly, biotinylated CD40 ECD-Fc were captured by streptavidin probe and the antibody association/dissociation were measured with light interferometry in realtime. KD were then calculated by Kₒₙ and K_{off}. The results are shown in the table below.

| Antibody Clone | K_{D} (pM) |
|---|---|
| 5 | 6.04 |
| 6 | 377 |
| 151 | 84 |
| 166 | 420 |
| 233 | 12 |
| APX005M | 41 |

### Example 5: Epitope Binning

Epitope binning was done using Octet system. Briefly, the first antibody was allowed to bind the CD40 ECD-Fc and the second antibody was introduced. The amount of second antibody binding was used to access the relationship between two binding epitopes. A corrected matrix showing the binning results is provided below. The results indicate that antibody clones 5 and 6 bind to different epitopes.

| Antibody | Clone 5 | Clone 6 | Control |
|---|---|---|---|
| Clone 5 | 0.0088 | 1.1051 | 0.018 |
| Clone 6 | 1.5388 | 0.0684 | 0.7226 |
| Control | 0.0862 | 0.1318 | 0.0241 |

### Example 6: Competition for CD40L Binding

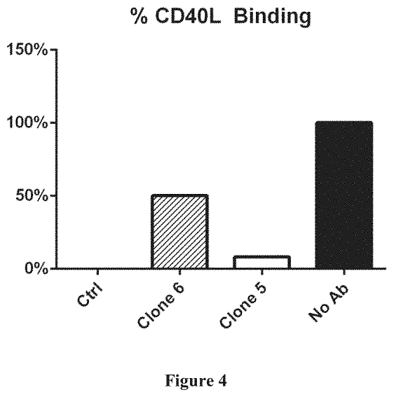

Competition ELISA were performed using CD40L and anti-CD40 antibodies. In brief, CD40L were coated on ELISA plate and exposed to pre-mixed CD40 ECD-Fc-His fusion protein and anti-CD40 antibody. CD40 binding to CD40L were then determined using anti-His antibody and presented as percentage of CD40 binding to CD40L where no antibody is present. As shown in FIG. 4, antibody clone 5 inhibits around 90% of CD40L binding while antibody clone 6 inhibits around 50%.

### Example 7: B Cell Activation

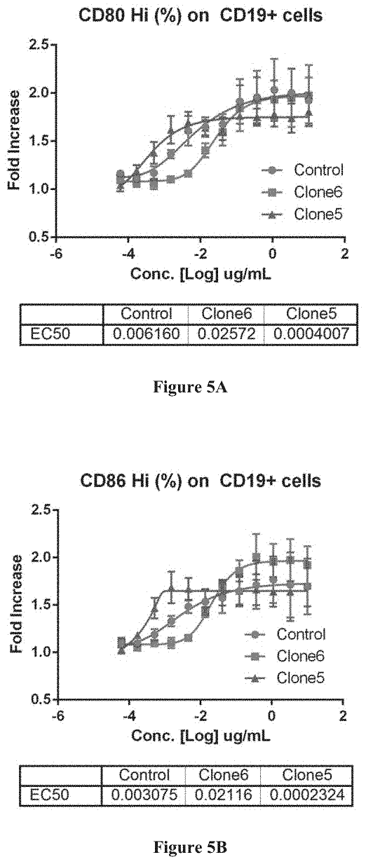

B cell activation by anti-CD40 antibodies were assessed using CD80 and CD86 expression. Briefly, healthy donor PBMC depleted of monocytes were incubated with IL-2 and IL-4 in the presence or absence of anti-CD40 antibodies for 48 hours. CD80 and CD86 expression on CD19⁺ cells were analyzed using flow cytometry. As shown in FIG. 5A, antibody clone 5 has an EC50 of 2.7 pM in activating B cell assessed by CD80 expression, antibody clone 6 has an EC50 of 0.17 nM, while the control antibody has an EC50 of 0.41 nM. As shown in FIG. 5B, antibody clone 5 has an EC50 of 1.5 pM in activating B cell assessed by CD86 expression, antibody clone 6 has an EC50 of 0.14 nM, while the control antibody has an EC50 of 21 pM. Notably, as shown in FIG. 5B, antibody clone 6 can activate B cell to a higher extent as compared to control and antibody clone 5, as indicated by the highest CD86 expression level at high concentration.

### Example 8: Dendritic Cell Maturation and Activation

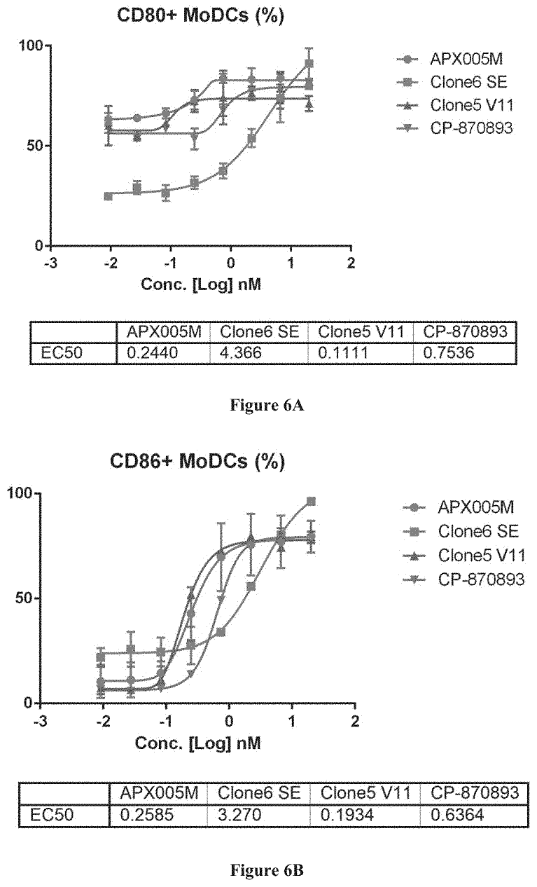

Dendritic cell maturation and activation by anti-CD40 antibodies were assessed using CD80 and CD86 expression. Briefly, monocytes were isolated from healthy donor PBMC, differentiation toward dendritic cells were induced with GM-CSF and IL-4 for 5 days. Anti-CD40 antibodies were then introduced for additional 48 hours. CD80 and CD86 expression MoDC were analyzed using flow cytometry. As shown in FIG. 6A, antibody clone 5 has an EC50 of 0.74 nM in activating monocyte derived dendritic cell (MoDC) assessed by CD80 expression, antibody clone 6 has an EC50 of 29 nM, while control antibody APX005M has an EC50 of 1.6 nM and control antibody CP-870893 (Creative Biolabs, Shirley, N.Y., USA) has an EC50 of 5.0 nM. As shown in FIG. 6B, antibody clone 5 has an EC50 of 1.3 nM in activating MoDC assessed by CD86 expression, antibody clone 6 has an EC50 of 22 nM, while control antibody APX005M has an EC50 of 1.7 nM and control antibody CP-870893 has an EC50 of 4.2 nM. Notably, as shown in FIGS. 6A and 6B, antibody clone 6 can activate MoDC to a higher extent as compared to the control antibodies and antibody clone 5, as indicated by the highest CD80 and CD86 expression level at high concentration.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An isolated anti-CD40 antibody or an antigen-binding fragment thereof comprising:
a) a heavy chain variable region comprising the complementarity determining regions (CDRs) of SEQ ID NOS: 13, 15 and 17 in that order; and a light chain variable region comprising the CDRs of SEQ ID NO: 14, 16 and 18 in that order; or
b) a heavy chain variable region comprising the CDRs of SEQ ID NOS: 19, 21 and 23 in that order; and a light chain variable region comprising the CDRs of SEQ ID NOS: 20, 22 and 24 in that order.

2. The antibody or an antigen-binding fragment thereof of claim 1, comprising
(a) a heavy chain variable region having an amino acid sequence at least about 90% identical to SEQ ID NO: 255; and a light chain variable region having an amino acid sequence at least about 90% identical to SEQ ID NO: 257;
(b) a heavy chain variable region having an amino acid sequence at least about 90% identical to SEQ ID NO: 259; and a light chain variable region having an amino acid sequence at least about 90% identical to SEQ ID NO: 261;
(c) a heavy chain variable region having an amino acid sequence at least about 90% identical to SEQ ID NO: 411; and a light chain variable region having an amino acid sequence at least about 90% identical to SEQ ID NO: 413; or
(d) a heavy chain variable region having an amino acid sequence at least about 90% identical to SEQ ID NO: 415; and a light chain variable region having an amino acid sequence at least about 90% identical to SEQ ID NO: 417.

3. The antibody or an antigen-binding fragment thereof of claim 1, comprising
(a) a heavy chain variable region having an amino acid sequence of SEQ ID NO: 255; and a light chain variable region having an amino acid sequence of SEQ ID NO: 257;
(b) a heavy chain variable region having an amino acid sequence of SEQ ID NO: 259; and a light chain variable region having an amino acid sequence of SEQ ID NO: 261;
(c) a heavy chain variable region having an amino acid sequence of SEQ ID NO: 411; and a light chain variable region having an amino acid sequence of SEQ ID NO: 413; or
(d) a heavy chain variable region having an amino acid sequence of SEQ ID NO: 415; and a light chain variable region having an amino acid sequence of SEQ ID NO: 417.

4. The antibody or antigen-binding fragment thereof of claim 1, wherein the antibody specifically blocks binding of CD40L to CD40.

5. The antibody or antigen-binding fragment thereof of claim 1, wherein the antibody is capable of activate B cells or dendritic cells.

6. (canceled)

7. The antibody or an antigen-binding fragment thereof of claim 1, which is chimeric or humanized.

8. The antibody or antigen-binding fragment thereof of claim 1, which is camelized single domain antibody, a diabody, a scFv, an scFv dimer, a BsFv, a dsFv, a (dsFv)₂, a dsFv-dsFv′, an Fv fragment, a Fab, a Fab′, a F(ab′)₂, a bispecific antibody, a ds diabody, a nanobody, a domain antibody, or a bivalent antibody.

9. (canceled)

10. The antibody or antigen-binding fragment thereof of claim 1, which is linked to one or more conjugate moieties, wherein the conjugate moiety comprises a clearance-modifying agent, a toxin, a detectable label a chemotherapeutic agent, or purification moiety.

11. (canceled)

12. An antibody or an antigen-binding fragment thereof which competes for the same epitope with the antibody or antigen-binding fragment thereof of claim 1.

13. A pharmaceutical composition comprising the antibody or antigen-binding fragment thereof of claim 1, and a pharmaceutically acceptable carrier.

14. An isolated polynucleotide encoding the antibody or an antigen-binding fragment thereof of claim 1.

15. A vector comprising the isolated polynucleotide of claim 14.

16. A host cell comprising the vector of claim 15.

17. A method of expressing a antibody or antigen-binding fragment thereof, comprising culturing the host cell of claim 16 under a condition at which the antibody or antigen-binding fragment thereof is expressed.

18. A method of treating a CD40 related disease or condition, comprising administering to the subject a therapeutically effective amount of the antibody or antigen-binding fragment thereof of claim 1.

19. (canceled)

20. The method of claim 18, wherein the disease or condition is cancer, autoimmune disease, inflammatory disease, or infectious disease.

21. The method of claim 20, wherein the cancer is adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalviolar cell lung cancer, mesothelioma, head and neck cancer, squamous cell carcinoma, lymphoma, lymphocytic leukemia, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer, and Hodgkin's Disease.

22-23. (canceled)

24. A method of modulating CD40 activity in a CD40-expressing cell, comprising exposing the CD40-expressing cell to the antibody or antigen-binding fragment thereof of claim 1.

25. A method of detecting presence or amount of CD40 in a sample, comprising contacting the sample with the antibody or antigen-binding fragment thereof of claim 1, and determining the presence or the amount of CD40 in the sample.

26. A method of diagnosing a CD40 related disease or condition in a subject, comprising: a) contacting a sample obtained from the subject with the antibody or antigen-binding fragment thereof of claim 1; b) determining presence or amount of CD40 in the sample; and c) correlating the presence or the amount of CD40 to existence or status of the CD40 related disease or condition in the subject.

27-29. (canceled)
